(19) United States
(12) Patent Application Publication
(10) Pub. No.: US 2006/0058269 A1
(43) Pub. Date: Mar. 16, 2006

(54) Carotenoid analogs or derivatives for the inhibition and amelioration of inflammation

(76) Inventors: Samuel Fournier Lockwood, Lago Vista, TX (US); Sean O'Malley, Honolulu, HI (US); Henry Jackson, Honolulu, HI (US); Geoff Nadolski, Kaneohe, HI (US)

Correspondence Address: MEYERTONS, HOOD, KIVLIN, KOWERT & GOETZEL, P.C., P.O. BOX 398, AUSTIN, TX, 78767-0398, US

(21) Appl. No.: 11/214,478

(22) Filed: Aug. 29, 2005

Related U.S. Application Data
(63) Continuation-in-part of application No. 11/106,378, filed on Apr. 14, 2005.
(60) Provisional application No. 60/562,195, filed on Apr. 14, 2004.

(51) Int. Cl. A61K 31/66; A61K 31/015

(52) U.S. Cl. 514/124; 514/763

(57) ABSTRACT

A method for inhibiting and/or ameliorating the occurrence of diseases in a human subject whereby a subject is administered a carotenoid analog or derivative, either alone or in combination with another carotenoid analog or derivative. In some embodiments, the administration of analogs or derivatives of carotenoids may inhibit and/or ameliorate the occurrence of diseases in subjects. In some embodiments, analogs or derivatives of carotenoids may be water-soluble and/or water dispersible. Maladies that may be treated with analogs or derivatives of carotenoids embodied herein may include diseases that provoke or trigger an inflammatory response. In an embodiment, asthma may be treated with analogs or derivatives of carotenoids embodied herein. In an embodiment, administering analogs or derivatives of carotenoids embodied herein to a subject may control or affect the bioavailability of eicosanoids. In an embodiment, atherosclerosis may be treated with analogs or derivatives of carotenoids embodied herein. In an embodiment, administering the analogs or derivatives of carotenoids embodied herein to a subject may control or affect the bioavailability of 5-LO-catalyzed eicosanoid metabolites. In an embodiment, 5-LO-catalyzed eicosanoid metabolites that may be controlled or affected by administering analogs or derivatives of carotenoids to a subject may include proinflammatory effector molecules (e.g., leukotrienes).

### PRIORITY CLAIM

This application is a continuation-in-part of and claims the benefit of priority under 35 U.S.C. §120 to U.S. patent application Ser. No. 11/106,378, entitled “CAROTENOID ANALOGS OR DERIVATIVES FOR THE INHIBITION AND AMELIORATION OF INFLAMMATION,” filed Apr. 14, 2005, which claims priority to Provisional Patent Application No. 60/562,195 entitled “CAROTENOID ANALOGS OR DERIVATIVES FOR THE INHIBITION AND AMELIORATION OF INFLAMMATION” filed on Apr. 14, 2004. The above-listed applications are commonly assigned with the present invention and the entire contents of which are incorporated herein by reference.

### BACKGROUND OF THE INVENTION

1. Field of the Invention

The invention generally relates to the fields of medicinal and synthetic chemistry. More specifically, the invention relates to the synthesis and use of carotenoid analogs or derivatives.

2. Description of the Relevant Art

Recent studies have demonstrated that initiation of lipid peroxidation and formation of bioactive eicosanoids are critical processes occurring in inflammation, particularly in both atherosclerosis and asthma (reactive airways disease; Zhang et al. 2002; Spanbroek et al. 2003; Dwyer et al. 2004; Helgadottir et al. 2004 are each incorporated herein). The current state of knowledge suggests that lipoxygenases, cyclooxygenases, and cytochrome P450 monooxygenases (CYPs) are the primary enzymatic participants in these lipid peroxidation events (Spector et al. 1988 (incorporated herein); Zhang et al. 2002). In addition, myeloperoxidase (MPO), a heme protein secreted by activated leukocytes, can also generate reactive intermediates that promote lipid peroxidation in vitro and in vivo (Baldus et al. 2003; Brennan et al. 2003 which are each incorporated herein).

In addition to their potent bronchoconstrictor properties, leukotrienes and other products of the 5-lipoxygenase pathway induce pathophysiologic responses similar to those associated with asthma. Specifically, 5-lipoxygenase products can cause tissue edema and migration of eosinophils and can stimulate airway secretions. The leukotrienes also stimulate cell cycling and proliferation of both smooth muscle and various hematopoietic cells; these observations provide further evidence of a potential role of leukotriene modifiers in altering the biology of the airway wall in asthma. Since all these responses contribute to asthma, the pharmaceutical industry initiated research programs to identify substances that could inhibit the action or synthesis of the leukotrienes.

Inhibition of 5-LO pathway activity in vivo will likely find application in those anti-inflammatory applications (e.g. atherosclerosis, asthma) for which downstream mediators of 5-LO activity (e.g. leukotriene B4 or LTB₄) are involved in the pathogenesis of disease.

### SUMMARY

In some embodiments, the administration of analogs or derivatives of carotenoids may inhibit and/or ameliorate the occurrence of certain maladies in subjects. Maladies that may be treated with analogs or derivatives of carotenoids embodied herein may include diseases that provoke, trigger or are associated with an inflammatory response. In some embodiments, analogs or derivatives of carotenoids may be water-soluble:

In an embodiment, at least a portion of the pathological complications associated with asthma may be ameliorated or inhibited in a patient by administering analogs or derivatives of carotenoids embodied herein.

In an embodiment, administering analogs or derivatives of carotenoids embodied herein to a subject may modulate or affect the bioavailability of eicosanoids.

In an embodiment, at least a portion of the pathological complications associated with atherosclerosis may be ameliorated or inhibited in a patient by administering analogs or derivatives of carotenoids embodied herein.

In an embodiment, administering the analogs or derivatives of carotenoids embodied herein to a subject may control or affect the bioavailability of 5-lipoxygenase (LO)-catalyzed eicosanoid metabolites. In an embodiment, 5-LO-catalyzed eicosanoid metabolites that may be controlled or affected by administering analogs or derivatives of carotenoids to a subject may include proinflammatory effector molecules (e.g., leukotrienes). Administration of analogs or derivatives of carotenoids according to the preceding embodiments may at least partially inhibit and/or influence the pathological complications associated with inflammation.

In some embodiments, the administration of structural analogs or derivatives of carotenoids may at least partially inhibit the biological activity of 5-LO. Inhibition of 5-LO activity may occur, at least in part, by forming a complex between a molecule of 5-LO and one or more molecules of the subject structural carotenoid analogs or derivatives.

In an embodiment, a composition is provided comprising the subject structural carotenoid analogs or derivatives contacted with 5-LO. The ratio of structural carotenoid analog or derivative contacted with 5-LO may range from about 0.1 to about 2.5. In some embodiments, the composition may be formed in a cell. In some embodiments, the composition may be formed in a cell, tissue or organ of a mammalian or human subject.

In some embodiments, the administration of structural analogs or derivatives of carotenoids by one skilled in the art—including consideration of the pharmacokinetics and pharmacodynamics of therapeutic drug delivery—is expected to inhibit and/or ameliorate disease conditions.

In some of the foregoing embodiments, analogs or derivatives of carotenoids administered to cells may be at least partially water-soluble.

Water-soluble carotenoid analogs or derivatives may have a water solubility of greater than about 1 mg/mL in some embodiments. In certain embodiments, water-soluble carotenoid analogs or derivatives may have a water solubility of greater than about 10 mg/mL. In some embodiments, water-soluble carotenoid analogs or derivatives may have a water solubility of greater than about 50 mg/mL.

In an embodiment, the administration of water soluble analogs or derivatives of carotenoids to a subject may inhibit and/or ameliorate some types of diseases that provoke or trigger an inflammatory response. In some embodiments, water-soluble analogs or derivatives of carotenoids may be administered to a subject alone or in combination with other carotenoid analogs or derivatives.

Embodiments may be further directed to pharmaceutical compositions comprising combinations of structural carotenoid analogs or derivatives to said subjects. The composition of an injectable structural carotenoid analog or derivative of astaxanthin may be particularly useful in the therapeutic methods described herein. In yet a further embodiment, an injectable astaxanthin structural analog or derivative is administered with another astaxanthin structural analog or derivative and/or other carotenoid structural analogs or derivatives, or in formulation with antioxidants and/or excipients that further the intended purpose. In some embodiments, one or more of the astaxanthin structural analogs or derivatives are water-soluble.

In some embodiments, the administration of structural analogs or derivatives of carotenoids by one skilled in the art—including consideration of the pharmacokinetics and pharmacodynamics of therapeutic drug delivery—is expected to inhibit and/or ameliorate disease conditions associated with elevated inflammation. In some of the foregoing embodiments, analogs or derivatives of carotenoids administered to cells may be at least partially water-soluble.

“Water-soluble” structural carotenoid analogs or derivatives are those analogs or derivatives that may be formulated in aqueous solution, either alone or with one or more excipients. Water-soluble carotenoid analogs or derivatives may include those compounds and synthetic derivatives that form molecular self-assemblies, and may be more properly termed “water dispersible” carotenoid analogs or derivatives. Water-soluble and/or “water-dispersible” carotenoid analogs or derivatives may be preferred in some embodiments.

Water-soluble carotenoid analogs or derivatives may have a water solubility of greater than about 1 mg/mL in some embodiments. In certain embodiments, water-soluble carotenoid analogs or derivatives may have a water solubility of greater than about 5 mg/ml-10 mg/mL. In certain embodiments, water-soluble carotenoid analogs or derivatives may have a water solubility of greater than about 20 mg/mL. In certain embodiments, water-soluble carotenoid analogs or derivatives may have a water solubility of greater than about 25 mg/mL. In some embodiments, water-soluble carotenoid analogs or derivatives may have a water solubility of greater than about 50 mg/mL.

In some embodiments, water-soluble analogs or derivatives of carotenoids may be administered to a subject alone or in combination with additional carotenoid analogs or derivatives.

In some embodiments, methods of modulating pathological complications associated with inflammation in a body tissue of a subject may include administering to the subject an effective amount of a pharmaceutically acceptable formulation including a synthetic analog or derivative of a carotenoid. The synthetic analog or derivative of the carotenoid may have the structure 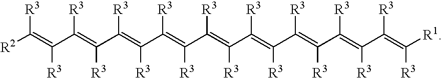
Each R³ may be independently hydrogen or methyl. R¹ and R² may be independently a cyclic ring including at least one substituent W or acyclic group including at least one substituent W. Each cyclic ring may be independently: 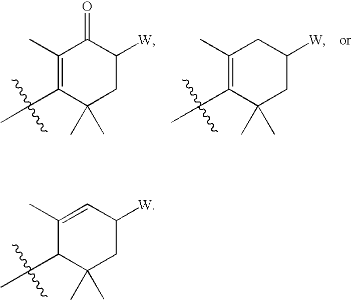
The acyclic group may have the structure

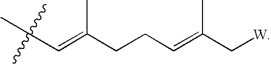

In some embodiments, at least one substituent W independently comprises 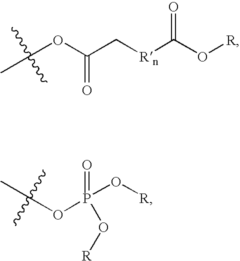
or a co-antioxidant. Each R′ may be CH₂. n may range from 1 to 9. Each R may be independently H, alkyl, aryl, benzyl, Group IA metal, or a co-antioxidant. Each co-antioxidant may be independently Vitamin C, Vitamin C analogs, Vitamin C derivatives, Vitamin E, Vitamin E analogs, Vitamin E derivatives, flavonoids, flavonoid derivatives, or flavonoid analogs. Flavonoids may include, for example, quercetin, xanthohumol, isoxanthohumol, or genistein.

In some embodiments, a method of treating inflammation may include administering to the subject an effective amount of a pharmaceutically acceptable formulation including a synthetic analog or derivative of a carotenoid. The synthetic analog or derivative of the carotenoid may have the structure 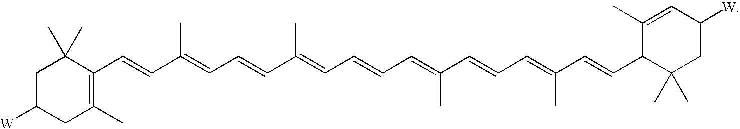
At least one substituent W may independently include 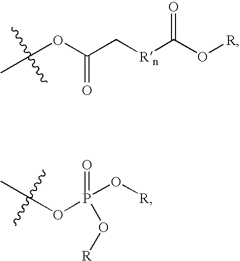
or a co-antioxidant. Each R′ may be CH₂. n may range from 1 to 9. Each R may be independently H, alkyl, aryl, benzyl, Group IA metal, or a co-antioxidant. Each co-antioxidant may be independently Vitamin C, Vitamin C analogs, Vitamin C derivatives, Vitamin E, Vitamin E analogs, Vitamin E derivatives, flavonoids, flavonoid analogs, or flavonoid derivatives. Flavonoids may include, for example, quercetin, xanthohumol, isoxanthohumol, or genistein.

In some embodiments, the carotenoid analog or derivative may have the structure 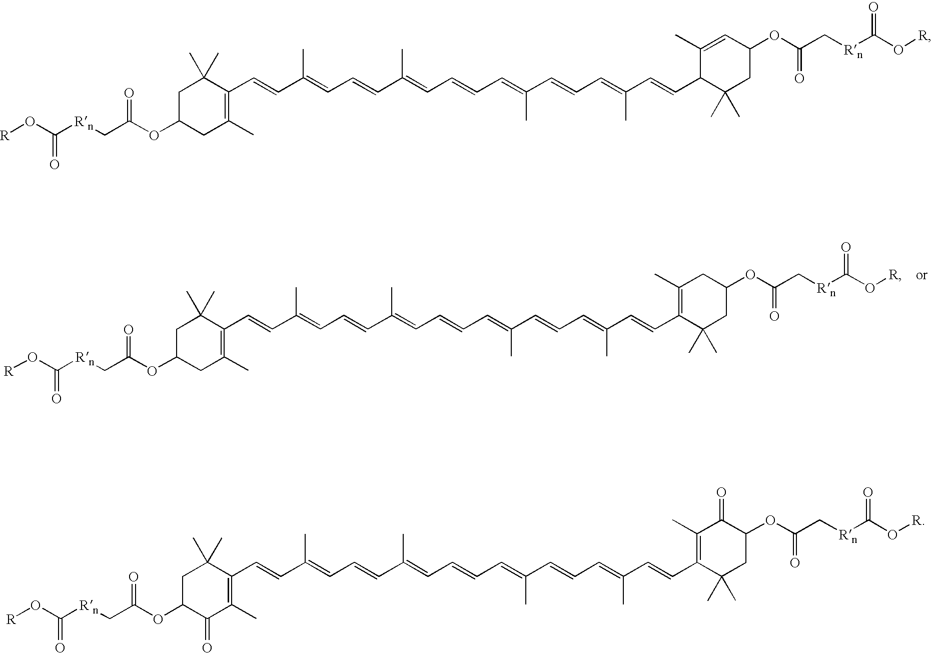
Each R′ may be CH₂. n may range from 1 to 9. Each R may be independently H, alkyl, aryl, benzyl, a Group IA metal (e.g., Na, K, Li or the like), or a co-antioxidant. Each co-antioxidant may be independently Vitamin C, Vitamin C analogs, Vitamin C derivatives, Vitamin E, Vitamin E analogs, Vitamin E derivatives, flavonoids, flavonoid analogs, or flavonoid derivatives. Flavonoids may include, for example, quercetin, xanthohumol, isoxanthohumol, or genistein. In an embodiment, R′ is CH₂, n is 1, and R is sodium.

In some embodiments, the carotenoid analog or derivative may have the structure 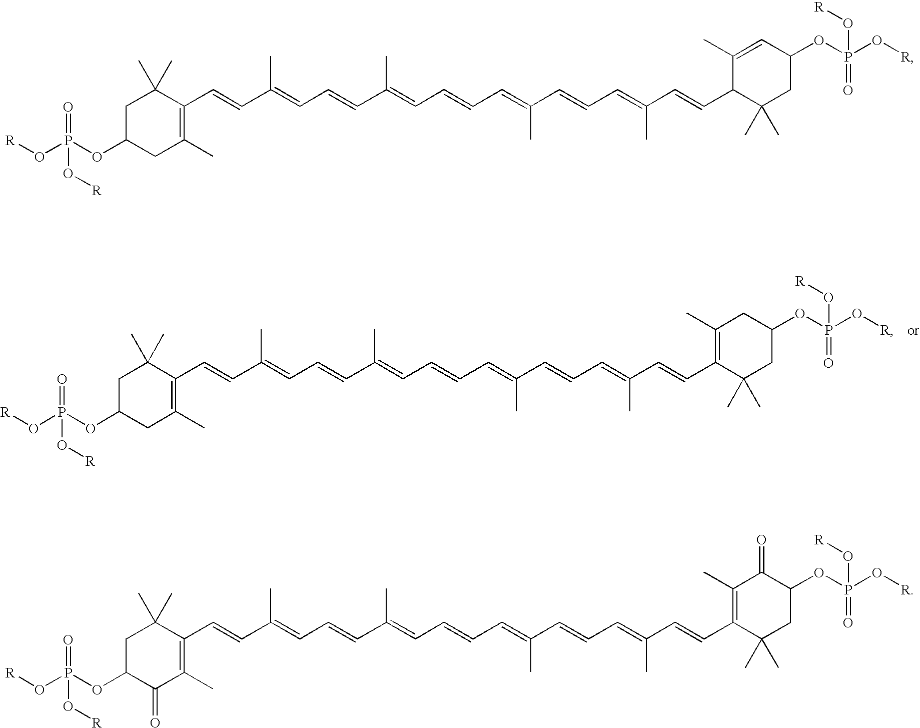
Each R may be independently H, alkyl, aryl, benzyl, a Group IA metal (e.g., Na, K, Li, or the like), or a co-antioxidant. Each co-antioxidant may be independently Vitamin C, Vitamin C analogs, Vitamin C derivatives, Vitamin E, Vitamin E analogs, Vitamin E derivatives, flavonoids, flavonoid analogs, or flavonoid derivatives. Flavonoids may include, for example, quercetin, xanthohumol, isoxanthohumol, or genistein. In an embodiment, R is sodium. When R includes Vitamin C, Vitamin C analogs, or Vitamin C derivatives, some embodiments may include carotenoid analogs or derivatives having the structure 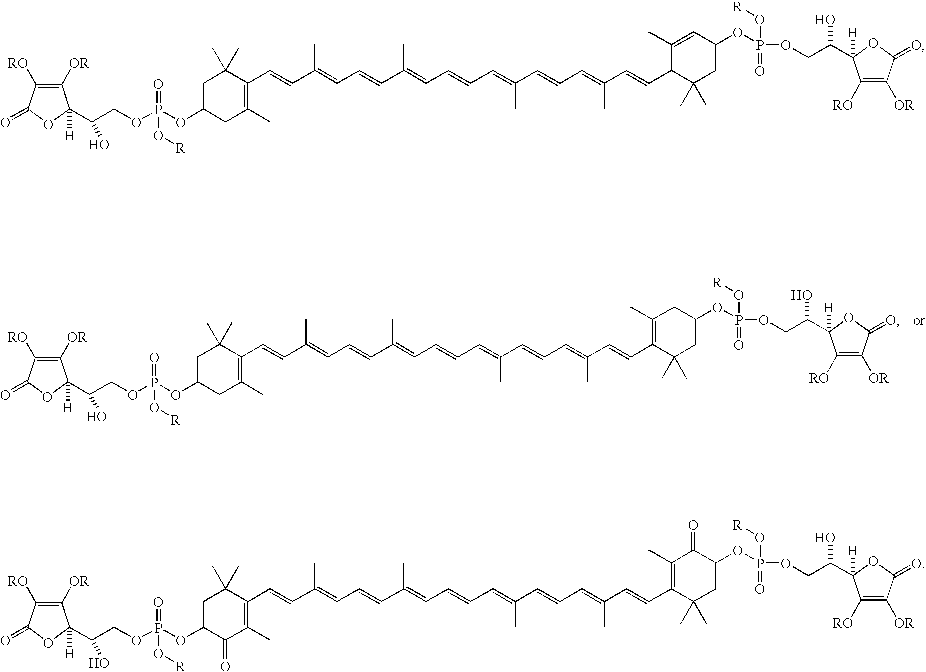
Each R may be independently H, alkyl, aryl, benzyl, or a Group IA metal. Certain embodiments may further directed to pharmaceutical compositions including combinations two or more structural carotenoid analogs or derivatives. Embodiments directed to pharmaceutical compositions may further include appropriate vehicles for delivery of said pharmaceutical composition to a desired site of action (i.e., the site a subject's body where the biological effect of the pharmaceutical composition is most desired). Pharmaceutical compositions including injectable structural carotenoid analogs or derivatives of astaxanthin, lutein or zeaxanthin may be particularly advantageous for the methods described herein. In yet a further embodiment, an injectable astaxanthin structural analog or derivative may be administered with a astaxanthin, zeaxanthin or lutein structural analog or derivative and/or other carotenoid structural analogs or derivatives, or in formulation with antioxidants and/or excipients that further the intended purpose. In some embodiments, one or more of the astaxanthin, lutein or zeaxanthin structural analogs or derivatives are water-soluble.

### BRIEF DESCRIPTION OF THE DRAWINGS

The above brief description as well as further objects, features and advantages of the methods and apparatus of the present invention will be more fully appreciated by reference to the following detailed description of presently preferred but nonetheless illustrative embodiments in accordance with the present invention when taken in conjunction with the accompanying drawings.

FIG. 1 is a depiction of several examples of “parent” carotenoid structures as found in nature.

FIG. 2 is a depiction of a time series of the UV/V s absorption spectra of the disodium disuccinate derivative of natural source lutein in water.

FIG. 3 is a depiction of a UV/V is absorption spectra of the disodium disuccinate derivative of natural source lutein in water (λₘₐₓ=443 nm), ethanol (λₘₐₓ=446 nm), and DMSO(λₘₐₓ=461 nm).

FIG. 4 is a depiction of a UV/V is absorption spectra of the disodium disuccinate derivative of natural source lutein in water (λₘₐₓ=442 nm) with increasing concentrations of ethanol.

FIG. 5 is a depiction of a time series of the UV/V is absorption spectra of the disodium diphosphate derivative of natural source lutein in water.

FIG. 6 is a depiction of a UV/V is absorption spectra of the disodium diphosphate derivative of natural source lutein in 95% ethanol (λₘₐₓ=446 nm), 95% DMSO (λₘₐₓ=459 nm), and water (λₘₐₓ=428 nm).

FIG. 7 is a depiction of a UV/V is absorption spectra of the disodium diphosphate derivative of natural source lutein in water (λₘₐₓ=428 nm) with increasing concentrations of ethanol.

FIG. 8 is a depiction of a mean percent inhibition (±SEM) of superoxide anion signal as detected by DEPMPO spin-trap by the disodium disuccinate derivative of natural source lutein (tested in water).

FIG. 9 is a depiction of a mean percent inhibition (±SEM) of superoxide anion signal as detected by DEPMPO spin-trap by the disodium diphosphate derivative of natural source lutein (tested in water).

FIG. 10 is a depiction of the chemical structures of three synthetic water-soluble carotenoid analogs or derivatives according to certain embodiments. (A) disuccinic acid astaxanthin ester; (B) disodium disuccinic acid ester astaxanthin salt (Cardax™); and (C) divitamnin C disuccinate astaxanthin ester.

FIG. 11 is a graphical depiction comparing the visible absorption spectra of meso-dAST in different solvents, in the presence and absence of 5-lipoxygenase (5-LO). 1: EtOH, c=7.1×10⁻⁶ M, 25° C. (right axis). 2: 0.1 M pH 8.0 Tris HCl buffer, c=1.2×10⁻⁵ M, 37° C. 3 and 4: conditions are the same as in ‘2’, except that 5-LO is present at the concentrations identified by the ligand/protein (L/P) ratios (inset). Molar absorption coefficients (ε) were calculated using the molar concentration of meso-dAST in the sample solutions.

FIG. 12 is a graphical depiction of a circular dichroism (CD) and visible absorption spectroscopic titration of 5-LO with meso-dAST at low L/P ratios (0.1 M, pH 8.0 Tris HCl buffer, [5-LOX]=4.5×10⁻⁵ M, 37° C.

FIG. 13 is a representative induced circular dichroism (CD) spectra and the corresponding visible absorption spectra obtained by titration of 5-LO with the meso-dAST carotenoid ligand at higher LIP values (0.1 M, pH 8.0 Tris HCl buffer, [5-LO]=1.2×10⁻⁵ M, 37° C.).

FIG. 14 depicts the determination of the association constant determination by curve fitting to the induced circular dichroism (CD) data obtained during titration of 5-LOX with meso-dAST. The sigmoidal curve was obtained using the “two binding sites” model (see text). The derived values of the association constants (K₁, K₂) are shown (inset).

FIG. 15 Is a depiction of the results obtained from computational docking of meso-dAST to 15-LOX. A). Space-fill-presentation of the best-energy model; the binding sites define a positive intermolecular overlay angle between the carotenoid ligands. B). Basic (red) and aromatic (green) residues of the binding site found within van der Waals contact (<4 Å) from the meso-dAST molecules (model ‘A’ and ‘B’).

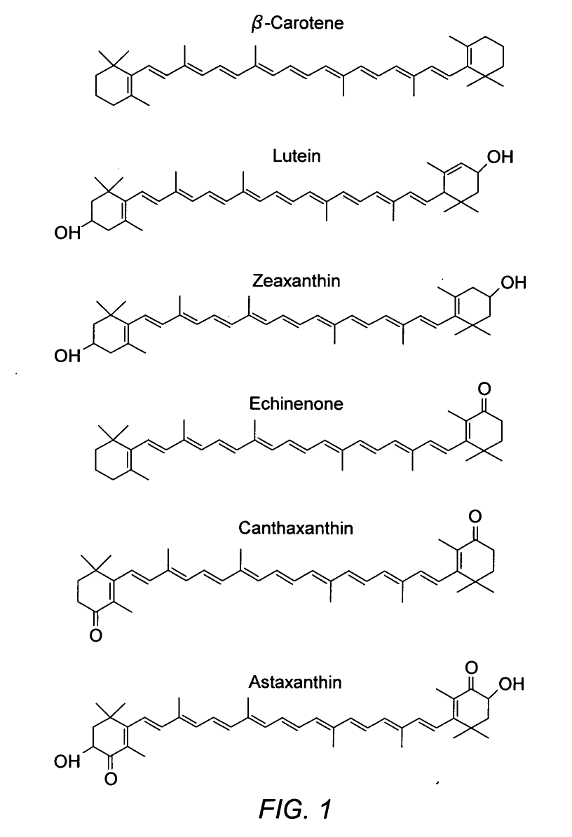

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION

### DEFINITIONS

In order to facilitate understanding of the invention, a number of terms are defined below. It will further be understood that, unless otherwise defined, all technical and scientific terminology used herein has the same meaning as commonly understood by a practitioner of ordinary skill in the art to which this invention pertains.

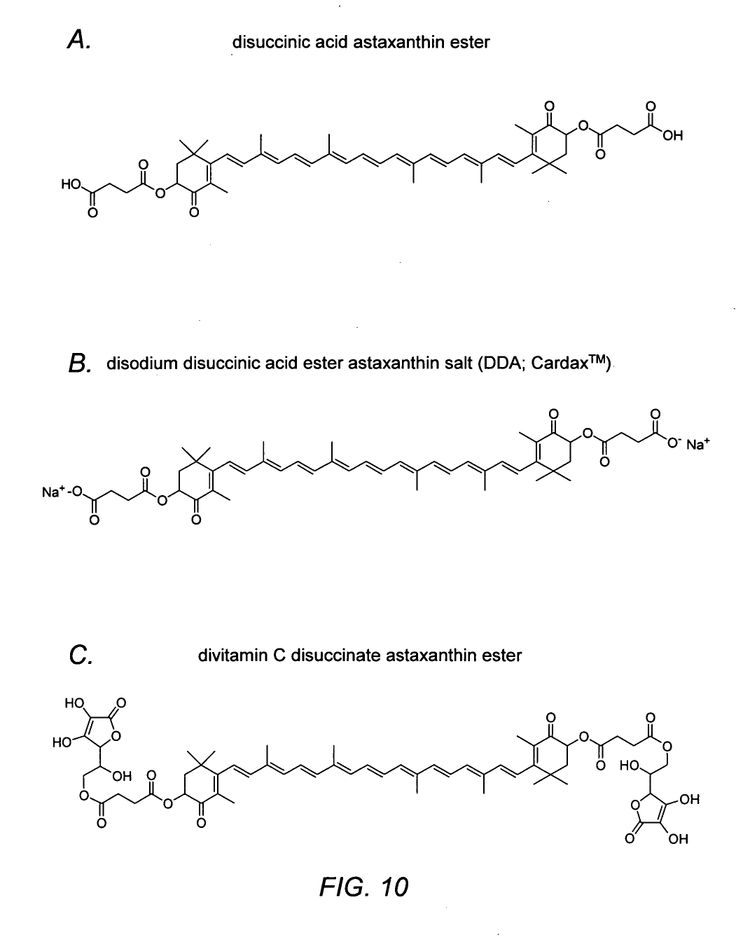

As used herein, terms such as “carotenoid analog” and “carotenoid derivative” generally refer to chemical compounds or compositions derived from a naturally occurring or synthetic carotenoid. Terms such as carotenoid analog and carotenoid derivative may also generally refer to chemical compounds or compositions that are synthetically derived from non-carotenoid based parent compounds; however, which ultimately substantially resemble a carotenoid derived analog. Non-limiting examples of carotenoid analogs and derivatives that may be used according to some of the embodiments described herein are depicted schematically in FIG. 10.

As used herein, the terms “disodium salt disuccinate astaxanthin derivative”, “dAST”, “ddAST”, “Cardax”, “Cardax™”, “rac”, “disodium disuccinate astaxanthin (DDA)”, and “astaxanthin disuccinate derivative (ADD)” represent varying nomenclature for the use of the disodium salt disuccinate astaxanthin derivative in various stereoisomer and aqueous formulations, and represent illustrative embodiments for the intended use of this structural carotenoid analog. The diacid disuccinate astaxanthin derivative (astaCOOH) is the protonated form of the derivative utilized for flash photolysis studies for direct comparison with non-esterified, “racemic” (i.e., mixture of stereoisomers) astaxanthin.

As used herein, the term “organ”, when used in reference to a part of the body of an animal or of a human generally refers to the collection of cells, tissues, connective tissues, fluids and structures that are part of a structure in an animal or a human that is capable of performing some specialized physiological function. Groups of organs constitute one or more specialized body systems. The specialized function performed by an organ is typically essential to the life or to the overall well being of the animal or human. Non-limiting examples of body organs include the heart, lungs, kidney, ureter, urinary bladder, adrenal glands, pituitary gland, skin, prostate, uterus, reproductive organs (e.g., genitalia and accessory organs), liver, gall-bladder, brain, spinal cord, stomach, intestine, appendix, pancreas, lymph nodes, breast, salivary glands, lacrimal glands, eyes, spleen, thymus, bone marrow. Non-limiting examples of body systems include the respiratory, circulatory, cardiovascular, lymphatic, immune, musculoskeletal, nervous, digestive, endocrine, exocrine, hepato-biliary, reproductive, and urinary systems. In animals, the organs are generally made up of several tissues, one of which usually predominates, and determines the principal function of the organ.

As used herein, the term “tissue”, when used in reference to a part of a body or of an organ, generally refers to an aggregation or collection of morphologically similar cells and associated accessory and support cells and intercellular matter, including extracellular matrix material, vascular supply, and fluids, acting together to perform specific functions in the body. There are generally four basic types of tissue in animals and humans including muscle, nerve, epithelial, and connective tissues.

As used herein, terms such as “biological availability,” “bioavailablity,” or the like generally refer to the relative amount of a biologically active factor or substance that is available to carry out a biological function.

As used herein, terms such as “inflammation,” “inflammatory response,” or the like, generally refer to an important biological process that is a component of the immune system. Inflammation is the first response of the immune system to infection, injury or irritation in a body. Though inflammation is an important component of innate immunity, if left unabated, it may result in severe and sometimes irreparable tissue damage. Inflammation also contributes to the pathophsiology of numerous disorders such as, for example, tissue reperfusion injury following myocardial infarction, system lupus erythematosis, Crohn's disease, asthma, atherosclerosis, and the like. An inflammatory response may include bringing leukocytes and plasma molecules to sites of infection or tissue injury. Inflammation may generally be characterized as causing a tissue to have one or more of the following charateristics: redness, heat, swelling, pain and dysfunction of the organs involved. At the tissue level, the principle effects of an inflammatory response may include increased vascular permeability, recruitment of leukocytes and other inflammatory cells to the site of the inflammatory response, changes in smooth muscle contraction and the synthesis and release of proinflammatory mediator molecules, including eicosanoids.

As used herein, the term “eicosanoid” generally refers to oxygenation products of long-chain fatty acids, including any of the physiologically active substances derived from arachidonic acid. Examples of eicosanoids include, but are not limited to, prostaglandins (PGs), prostacyclins (PCs), leukotrienes (LTs), epoxyeicosatrienoic acids (EETs), and thromboxanes (TXs). Further examples of eicosanoids include those intermediate metabolites that are part of the synthetic pathways of prostaglandins, prostacyclins, leukotrienes, EETs and thromboxanes such as, for example, HETEs, HPETEs, isoprostanes, HODEs, and other such intermediate metabolites that would be readily recognized by an ordinary practitioner of the art.

As used herein, the term “lipoxygenase”, or “LO” generally refers to a class of enzymes that catalyze the oxidative conversion of arachidonic acid to the hydroxyeicosetrinoic acid (HETE) structure in the synthesis of leukotrienes. The term “5-lipoxygenase”, or “5-LO” generally refers to one member of this class of enzymes that has lipoxygenase and dehydrase activity, and that catalyzes the conversion of arachidonic acid to 5-hydroperoxyeicatetraenoic acid (HPETE) and leukotriene A₄ (LTA₄).

As used herein, the term “leukotriene”, or “LT” generally refers to any of several physiologically active lipid compounds that contain 20 carbon atoms, are related to prostaglandins, and mediate an inflammatory response. Leukotrienes are eicosanoids that are generated in basophils, mast cells, macrophages, and human lung tissue by lipoxygenase-catalyzed oxygenation of long-chain fatty acids, especially of arachidonic acid, and that participate in allergic responses (as bronchoconstriction in asthma). Exemplary leukotrienes include LTA₄, LTC₄, LTD₄, LTE₄ and the lipoxins (LXs).

The term “modulate,” as used herein, generally refers to a change or an alteration in a biological parameter. Examples of biological parameters subject to modulation according to certain embodiments described herein may include, by way of non-limiting example only: inflammation, initiation of an inflammatory reaction, enzymatic activity, protein expression, cellular activity, production of hormonal intermediates, the relative levels of hormones or effector molecules such as, for example, eicosanoids, leukotrienes, prostaglandins, or intermediates thereof, or the like. “Modulation” may refer to a net increase or a net decrease in the biological parameter. Furthermore, it will be readily apparent to one of ordinary skill in the art that modulating a biological parameter can, in some instances, affect biological processes that themselves depend on that parameter. As used herein the terms “inhibiting” and “ameliorating,” when used in the context of modulating a pathological or disease state, generally refers to the prevention and/or reduction of at least a portion of the negative consequences of the disease state. When used in the context of biochemical pathway or of protein function, the term “inhibiting” generally refers to a net reduction in the activity of the pathway or function.

As used herein the terms “subject” generally refers to a mammal, and in particular to a human.

As used herein the terms “administering,” when used in the context of providing a pharmaceutical composition to a subject generally refers to providing to the subject one or more pharmaceutical, “over-the-counter” (OTC) or nutraceutical compositions via an appropriate delivery vehicle such that the administered compound achieves one or more biological effects for which the compound was administered. By way of non-limiting example, a composition may be administered parenteral, subcutaneous, intravenous, intracoronary, rectal, intramuscular, intra-peritoneal, transdermal, or buccal routes of delivery. The dosage of pharmacologically active compound that is administered will be dependent upon the age, health, weight, and/or disease state of the recipient, concurrent treatments, if any, the frequency of treatment, and/or the nature and magnitude of the biological effect that is desired.

As used herein, the term “polypeptide” generally refers to a naturally occurring, recombinant or synthetic polymer of amino acids, regardless of length or post-translational modification (e.g., cleavage, phosphorylation, glycosylation, acetylation, methylation, isomerization, reduction, farnesylation, etc . . . ), that are covalently coupled to each other by sequential peptide bonds. Although a “large” polypeptide is typically referred to in the art as a “protein” the terms “polypeptide” and “protein” are often used interchangeably. As used herein, the term “substantially identical”, when used in reference to a polynucleotide, generally refers to a polynucleotide, or a portion or fragment thereof, whose nucleotide sequence is at least 95%, 90%, 85% 80%, 70%, 60% or 50% identical to the nucleotide sequence of a reference polynucleotide. When used in reference to a polypeptide, the term generally refers to a polypeptide, or a fragment thereof, whose amino acid sequence is at least 95%, 90%, 85% 80%, 70%, 60% or 50% identical to the amino acid sequence of a reference polypeptide. For polypeptides, the length of comparison sequences will generally at least about 5 amino acids, and may include the complete polypeptide sequence. For nucleic acids, the length of comparison sequences will generally be at least about 15 nucleotides, and may include the complete reference nucleic acid sequence. Sequence identity between two or more polypeptide or nucleic acid sequences is typically determined using sequence analysis software (e.g., Sequence Analysis Software Package of the Genetics Computer Group, University of Wisconsin Biotechnology Center) designed for this purpose. Such software matches similar sequences by assigning degrees of homology to various substitutions, deletions, substitutions, and other modifications. Conservative substitutions typically include substitutions within the following groups: Gly; Ala; Val, Ile, Leu; Asp, Glu, Asn, Gln; Ser, Thr; Lys, Arg; and Phe, Tyr.

The term “portion”, as used herein, in the context of a molecule, such as a polypeptide or of a polynucleotide (as in “a portion of a given polypeptide/polynucleotide”) generally refers to fragments of that molecule. The fragments may range in size from three amino acid or nucleotide residues to the entire molecule minus one amino acid or nucleotide. Thus, for example, a polypeptide “comprising at least a portion of the polypeptide” encompasses the polypeptide and/or fragments thereof, including but not limited to the entire polypeptide minus one amino acid.

As used herein, terms such as “pharmaceutical composition,” “pharmaceutical formulation,” “pharmaceutical preparation,” or the like, generally refer to formulations that are adapted to deliver a prescribed dosage of one or more pharmacologically active compounds to a cell, a group of cells, an organ or tissue, an animal or a human. The determination of an appropriate prescribed dosage of a pharmacologically active compound to include in a pharmaceutical composition in order to achieve a desired biological outcome is within the skill level of an ordinary practitioner of the art. Pharmaceutical preparations may be prepared as solids, semi-solids, gels, hydrogels, liquids, solutions, suspensions, emulsions, aerosols, powders, or combinations thereof. Included in a pharmaceutical preparation may be one or more carriers, preservatives, flavorings, excipients, coatings, stabilizers, binders, solvents and/or auxiliaries. Methods of incorporating pharmacologically active compounds into pharmaceutical preparations are widely known in the art.

A “pharmaceutically acceptable formulation,” as used herein, generally refers to a non-toxic formulation containing a predetermined dosage of a pharmaceutical composition, wherein the dosage of the pharmaceutical composition is adequate to achieve a desired biological outcome. A component of a pharmaceutically acceptable formulation may generally include an appropriate delivery vehicle that is suitable for the proper delivery of the pharmaceutical composition to achieve the desired biological outcome.

As used herein the term “antioxidant” may be generally refer to any one or more of various substances (as beta-carotene, vitamin C, and α-tocopherol) that inhibit oxidation or reactions promoted by Reactive Oxygen Species (ROS) and other radical and non-radical species.

As used herein the term “co-antioxidant” may be generally defined as an antioxidant that is used and that acts in combination with another antioxidant (e.g., two antioxidants that are chemically and/or functionally coupled, or two antioxidants that are combined and function with each another in a pharmaceutical preparation). The effects of co-antioxidants may be additive (i.e., the anti-oxidative potential of one or more anti-oxidants acting additively is approximately the sum of the oxidative potential of each component anti-oxidant) or synergistic (i.e., the anti-oxidative potential of one or more anti-oxidants acting synergistically may be greater than the sum of the oxidative potential of each component anti-oxidant).

Compounds described herein embrace isomers mixtures, racemic, optically active, and optically inactive stereoisomers and compounds.

Eicosanoids and Inflammation

Eicosanoids are a class of lipid-based hormones that are derived from the oxidation of polyunsaturated long chain fatty acids (e.g., linoleic and arachidonic acid). Arachidonic acid (AA), also known as arachidonate, is the most abundant and physiologically important eicosanoid precursor. The immediate cellular precursor to AA is linoleic acid (LA). Oxidation of AA by enzymes of cyclooxygenase (COX), lipoxygenase (LO) or cytochrome-P450 monooxygenase (CYP) families results in the formation of prostaglandins (PG), leukotrienes (LT) and epoxyeicosatrienoic acids (EETs), respectively. Eicosanoids may also arise through the non-enzymatic oxidation of AA. Exemplary though non-limiting eicosanoids arising through non-enzymatic oxidation of AA include the F₂-isoprostanoids (e.g. 8-iso-F2α) and 9-hydroxyeicosatetraenoic acid (HETE). Eicosanoids regulate many cellular functions and play crucial roles in a variety of physiological and pathophysiological processes, including for example regulation of smooth muscle contractility and various immune and inflammatory functions.

Hydroxyeicosatetraenoic Acids

The hydroxyeicosatetraenoic acids (HETEs) are products of arachidonic acid metabolism that are derived primarily from the lipoxygenase pathways. Lipoxygenases convert AA first to a hydroperoxyeicosatetraenoic acid (HPETE); subsequently, the hydroperoxy group is reduced, forming the corresponding HETE. 5-, 12-, and 15-HETE constitute the main forms of HETE. Other isomers, including 8-, 9-, 11-, 19-, and 20-HETE have also been routinely detected. There is substantial evidence suggesting that 11- and 15-HETE may be produced by cyclooxygenase enzymes (e.g. COX-1; Bailey et al. 1983). Besides the well-known effect on chemotaxis of neutrophils, a number of other important actions have been attributed to HETEs, including modulation of intracellular calcium concentration, cell proliferation, prostaglandin formation, and other pro-inflammatory activities (Spector et al. 1988). Hydroxy-octadecadienoic acids (HODEs) are products of linoleic acid metabolism. As LA comprises ˜40-45% of the polyunsaturated fatty acids in LDL, HODEs are the most abundant oxidation products in atherosclerotic plaque (Waddington et al. 2001). A non-specific stereoisomeric pattern of oxidative modification is seen with HODEs, suggesting a non-enzymatic production of these markers in vivo (Waddington et al. 2001).

5-Lipoxygenase (5-LO) in cardiovascular Disease

The identification of 5-lipoxygenase (5-LO) as a major gene contributing to atherosclerosis susceptibility in mice—with decreased activity reflected in substantially reduced lesion formation—established the mouse model as an appropriate model system for evaluation of specific pro-inflammatory mediators in cardiac disease (Mehrabian et al. 2002). Subsequently, the 5-LO pathway was demonstrated to be abundantly expressed in the arterial walls of human patients afflicted with various lesion stages of atherosclerosis of the aorta, coronary, and carotid arteries (Spanbroek et al. 2003), and is currently being pursued as the “5-LO atherosclerosis hypothesis” (Lötzer et al. 2005). The emerging data support a model of atherogenesis in which 5-LO cascade-dependent inflammatory circuits (consisting of several leukocyte lineages) contribute to pathology within the vessel wall during critical stages of lesion development. Most recently, Cipollone and colleagues demonstrated the first association between 5-LO expression and atherosclerotic plaque instability in humans (Cipollone et al. 2005). They have proposed 5-LO as a marker for increased risk of acute ischemic cardiovascular events, and suggest that the 5-LO/leukotriene pathway should provide a novel therapeutic approach for plaque stabilization and prevention of acute coronary syndromes.

Leukotrienes and the 5-Lipoxygenase Pathway

The first step in the enzymatic synthesis of leukotrienes is catalyzed by LO enzymes. Mammals express a family of LO enzymes that catalyze the ultimate oxygenation of AA to leukotrienes at different sites. The products of LO catalysis have numerous important physiological functions.

The most widely studied leukotrienes are those whose production is acatalyzed by the 5-Lipoxygenase (5-LO) pathway. 5-LO is expressed in the cytosol of leukocytes, including basophils, Mast cells, eosinophils, monocytes and macrophages, where the enzyme catalyzes the conversion of arachidonate to 5-HPETE (5-hydroperoxyeicosatetraenoic acid). 5-HPETE is then converted to various leukotrienes that cause inflammation and asthmatic constriction of the bronchioles. Leukotrienes participate in numerous physiological processes, which may include host defense reactions and pathophysiological conditions such as immediate hypersensitivity and inflammation. Leukotrienes may have potent actions on many essential organs and systems, which may include the cardiovascular, pulmonary, and central nervous system as well as the gastrointestinal tract and the immune system.

The metabolism of AA by the enzymes 5-, 12-, and 15-LO results in the production of HPETEs, which may be converted to hydroxyl derivatives HETEs or LTs. The most widely investigated LO metabolites are the leukotrienes produced by 5-LO. 5-LO is an enzyme expressed in cells capable of eliciting inflammatory responses in mammals, such as polymorphonuclear (PMNs) cells, basophils, mast cells, eosinophils, monocytes/macrophages and epithelial cells. 5-LO requires the presence of the membrane protein 5-Lipoxygenase-activating protein (FLAP). FLAP binds AA, facilitating its interaction with the 5-LO. 5-LO, FLAP, and Phospholipase A₂ (which catalyzes release of arachidonate from phospholipids) form a complex in association with the nuclear envelope during leukotriene synthesis in leukocytes. The 5-LO pathway is of great clinical significance, since it may be associated with inflammatory disorders such as asthma or atherosclerosis. 5-LO oxidizes AA to form 5-hydroperoxyeicosatetraenoic acid (HPETE). 5-HPETE may then be further reduced to for 5-HETE or the intermediate leukotriene LTA4. LTA4 may then be catalyzed into the effector molecules LTB₄ through the action of a hydrolase, or to LTC₄, LTD₄, and LTE₄ through the action of glutathione-S-transferase, or acted on by other lipoxygenases to form lipoxins. The various LO pathways and leukotriene biosynthetic pathways are discussed in detail in Drazen et al., 1999 and Spector et al., 1988, both of which are incorporated by reference as though fully set forth herein. LTB₄ is a potent inducer of leukocyte chemotaxis and aggregation, vascular permeability, lymphocyte proliferation and the secretion of immuno-modulatory cytokines which may include interferon (IFN)-γ, inteleukin (IL)-1 and IL-2. LTC₄, LTD₄, and LTE₄ increase vascular permeability, are potent bronchoconstrictors and are components of the slow-reacting substance of anaphylaxis (SRS-A), which is secreted during asthmatic and anaphylactic episodes.

Because of the function of leukotrienes as proinflammatory hormones, it may be desirable to develop anti-leukotriene therapies as potential treatments for maladies that may be in part attributable to the induction of an inflammatory response, such as asthma or atherosclerosis. Strategies to reduce the biological availability of leukotrienes may include the development of 5-lipoxygenase inhibitors, leukotrienene receptor antagonists, inhibitors of FLAP, or inhibitors of phospholipase-A₂, which catalyzes the production of AA.

Anti-leukotriene therapies may include therapies that modulate 5-LO function. As used herein therapies that “modulate 5-LO function” may include for example therapies that modulate 5-LO enzyme activity, 5-LO expression, 5-LO stability, 5-LO cellular localization, and/or any other means of controlling the biological activity of the 5-LO pathway in vivo such that the biological availability of metabolized synthesized by 5-LO catalysis is at least partially reduced.

In some embodiments, administration of analogs or derivatives of carotenoids embodied herein to a subject may reduce the severity of an inflammatory response. In an embodiment, administering the analogs or derivatives of carotenoids embodied herein to a subject may reduce the severity of an asthmatic episode in a subject. In an embodiment, administering the analogs or derivatives of carotenoids embodied herein to a subject may reduce the severity of atherosclerosis in a subject. In an embodiment, administering the analogs or derivatives of carotenoids embodied herein to a subject may control the biological availability of arachidonic acid, linoleic acid and/or eicosanoids that are synthesized therefrom. In an embodiment, administering the analogs or derivatives of carotenoids embodied herein to a subject may substantially reduce the biological availability of 5-lipoxygenase (5-LO)-catalyzed eicosanoids including, but not limited to, leukotrienes (LTs)-A₄, B₄, C₄, D₄ and E₄, and/or other eicosanoids that result from 5-LO catalytic activity.

In certain embodiments, the biological activity of 5-LO may be modulated by contacting 5-LO, or a portion of fragment thereof with the subject carotenoid analogs or derivatives. Without being bound by any particular theory or mechanism of action, forming such complexes may reduce, inhibit or otherwise alter that activity of 5-LO and/or the biological availability of eicosanoids resulting from 5-LO activity.

In some embodiments, administering the analogs or derivatives of carotenoids embodied herein to a subject may modulate the biological availability of certain oxidative stress markers. In an embodiment, such activity may be manifested as a general sparing effect on AA and LA eicosanoid substrates (see for example, Table 3). By way of example, administering the subject carotenoid analogs or derivatives to a cell, a tissue or a subject may reduce the biological availability of F₂-isoprostanes. F₂-isoprostanes are prostaglandin-like products of free radical-catalyzed AA peroxidation, and are established biomarkers of in vivo lipid peroxidation (Singh et al. 2005). In addition, they can, in certain circumstances, exert physiological and/or pathophysiological effects such as vasoconstriction in the in vivo setting (Roberts and Morrow 2002). In yet another non-limiting example, administration of the subject carotenoid analogs or derivatives may reduce the biological availability of 8-iso-F_{2α} (see below). Reduction of levels of the aforementioned compounds support the role for the presently described structural carotenoid analogs and derivatives as anti-inflammatory and antioxidant compounds.

In some embodiments, administering the analogs or derivatives of carotenoids embodied herein may reduce the biological availability of the proinflammatory factor, prostaglandin F_{2α} (PGF_{2α}). This product of the cyclooxygenase (COX) enzyme can also be modulated by COX-inhibitors such as aspirin (Helmersson et al. 2005).

In some embodiments, administering the analogs or derivatives of carotenoids embodied herein may modulate the specific activity 5-LO enzymatic activity in vivo. In one non-limiting example, disclosed in more detail below, specific activity against relevant enzymatic activity in vivo is obtained for 5-HETE and its oxidative product, 5-oxo-ETE, at the time point of maximal monocyte/macrophage recruitment 72 h thioglycollate/4 h zymosan (results summarized in Table 3). Again, without being bound to a particular theory or mechanism of action, the data disclosed herein support direct effect of the subject carotenoids on 5-LO activity in vivo. In some embodiments, the effect of the subject carotenoids on 5-LO enzyme activity may be mediated, at least in part, by the ability of the carotenoid analogs or derivatives to bind to and form higher order molecular complexes with 5-LO. Moreover, administration of the subject carotenoids may, in certain embodiments, reducte the biological availability of 11-HETE and/or 9-HODE (as was observed in a rat experimental infarction model, Gross et al. 2005; definitive identification in human coronary plaque, Kühn et al. 1992). The demonstration of activity against 11-HETE-together with PGF_{2α}, products of cyclooxygenase enzyme activity—supports model system studies of non-esterified astaxanthin in infectious disease, in which COX activity was reduced in a mouse infectious disease model (Lee et al. 2003). The activity of the subjects carotenoid analogs and derivatives against 5-LO described herein thus represents the first such demonstration for a carotenoid derivative.

Synthesis nd Properties of Structural Carotenoid Analogs and Derivatives

In some embodiments, carotenoid analogs or derivatives may be employed in “self-formulating” aqueous solutions, in which the compounds spontaneously self-assemble into macromolecular complexes. These complexes may provide stable formulations in terms of shelf life. The same formulations may be parenterally administered, upon which the spontaneous self-assembly is overcome by interactions with serum and/or tissue components in vivo.

Some specific embodiments may include phosphate derivatives, succinate derivatives, co-antioxidant derivatives (e.g., Vitamin C, Vitamin C analogs, Vitamin C derivatives, Vitamin E, Vitamin E analogs, Vitamin E derivatives, flavonoids, flavonoid analogs, or flavonoid derivatives), or combinations thereof derivatives or analogs of carotenoids. Flavonoids may include, for example, quercetin, xanthohumol, isoxanthohumol, or genistein. Derivatives or analogs may be derived from any known carotenoid (naturally or synthetically derived). Specific examples of naturally occurring carotenoids which compounds described herein may be derived from include for example zeaxanthin, lutein, lycophyll, astaxanthin, and lycopene.

In some embodiments, one or more co-antioxidants may be coupled to a carotenoid or carotenoid derivative or analog.

The synthesis of water-soluble and/or water-dispersible carotenoids (e.g., C40) analogs or derivatives—as potential parenteral agents for clinical applications may improve the injectability of these compounds as therapeutic agents, a result perhaps not achievable through other formulation methods. The methodology may be extended to carotenoids with fewer than 40 carbon atoms in the molecular skeleton and differing ionic character. The methodology may be extended to carotenoids with greater than 40 carbon atoms in the molecular skeleton. The methodology may be extended to non-symmetric carotenoids. The aqueous dispersibility of these compounds allows proof-of-concept studies in model systems (e.g. cell culture), where the high lipophilicity of these compounds previously limited their bioavailability and hence proper evaluation of efficacy. Esterification or etherification may be useful to increase oral bioavailability, a fortuitous side effect of the esterification process, which can increase solubility in gastric mixed micelles. The net overall effect is an improvement in potential clinical utility for the lipophilic carotenoid compounds as therapeutic agents.

In some embodiments, the principles of retrometabolic drug design may be utilized to produce novel soft drugs from the asymmetric parent carotenoid scaffold (e.g., RRR-lutein (β, ε-carotene-3,3′-diol)). For example, lutein scaffold for derivatization was obtained commercially as purified natural plant source material, and was primarily the RRR-stereoisomer (one of 8 potential stereoisomers). Lutein (Scheme 1) possesses key characteristics—similar to starting material astaxanthin—which make it an ideal starting platform for retrometabolic syntheses: (1) synthetic handles (hydroxyl groups) for conjugation, and (2) an excellent safety profile for the parent compound. As stated above, lutein is available commercially from multiple sources in bulk as primarily the RRR-stereoisomer, the primary isomer in the human diet and human retinal tissue.

In some embodiments, carotenoid analogs or derivatives may have increased water solubility and/or water dispersibility relative to some or all known naturally occurring carotenoids. Contradictory to previous research, improved results are obtained with derivatized carotenoids relative to the base carotenoid, wherein the base carotenoid is derivatized with substituents including hydrophilic substituents and/or co-antioxidants.

In some embodiments, the carotenoid derivatives may include compounds having a structure including a polyene chain (i.e., backbone of the molecule). The polyene chain may include between about 5 and about 15 unsaturated bonds. In certain embodiments, the polyene chain may include between about 7 and about 12 unsaturated bonds. In some embodiments a carotenoid derivative may include 7 or more conjugated double bonds to achieve acceptable antioxidant properties.

In some embodiments, decreased antioxidant properties associated with shorter polyene chains may be overcome by increasing the dosage administered to a subject or patient.

In some embodiments, a chemical compound including a carotenoid derivative or analog may have the general structure (126): 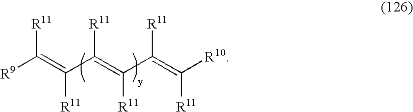
Each R¹¹ may be independently hydrogen or methyl. R⁹ and R¹⁰ may be independently H, an acyclic alkene with one or more substituents, or a cyclic ring including one or more substituents. y may be 5 to 12. In some embodiments, y may be 3 to 15. In certain embodiments, the maximum value of y may only be limited by the ultimate size of the chemical compound, particularly as it relates to the size of the chemical compound and the potential interference with the chemical compound's biological availability as discussed herein. In some embodiments, substituents may be at least partially hydrophilic. These carotenoid derivatives may be included in a pharmaceutical composition.

In some embodiments, the carotenoid derivatives may include compounds having the structure (128): 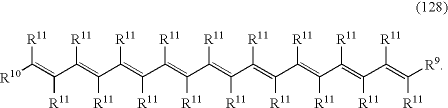
Each R¹¹ may be independently hydrogen, methyl, alkyl, alkenyl, or aromatic substituents. R⁹ and R¹⁰ may be independently H, an acyclic alkene with at least one substituent, or a cyclic ring with at least one substituent having general structure (130): 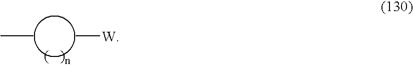
where n may be between 4 to 10 carbon atoms. W is the substituent.

In some embodiments, each cyclic ring may be independently two or more rings fused together to form a fused ring system (e.g., a bi-cyclic system). Each ring of the fused ring system may independently contain one or more degrees of unsaturation. Each ring of the fused ring system may be independently aromatic. Two or more of the rings forming the fused ring system may form an aromatic system.

In some embodiments, a chemical composition may include a carotenoid derivative having the structure 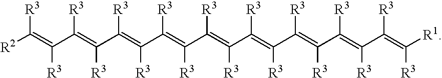
Each R³ may be independently hydrogen or methyl. R¹ and R² may be a cyclic ring including at least one substituent. Each cyclic ring may be independently: 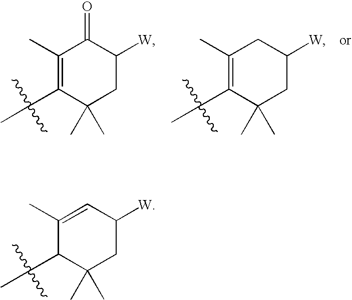
W is the substituent. In some embodiments R¹ and R² may be an acyclic group including at least one substituent. Each acyclic may be:

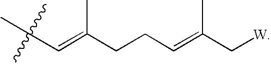

In some embodiments, a chemical composition may include a carotenoid derivative having the structure 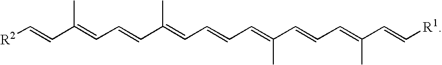
R¹ and R² may be a cyclic ring including at least one substituent. Each cyclic ring may be independently: 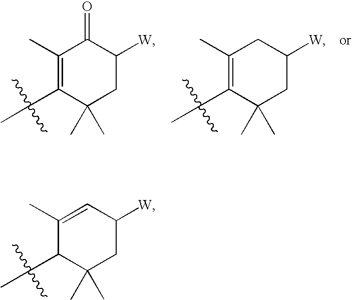
where W is the substituent. In some embodiments R¹ and R² may be an acyclic group including at least one substituent. Each acyclic group may be:

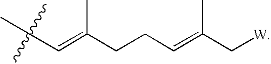

In some embodiments, a method of treating a proliferative disorder may include administering to the subject an effective amount of a pharmaceutically acceptable formulation including a synthetic analog or derivative of a carotenoid. The synthetic analog or derivative of the carotenoid may have the structure 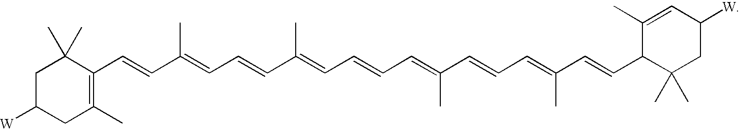
At least one substituent W may independently include 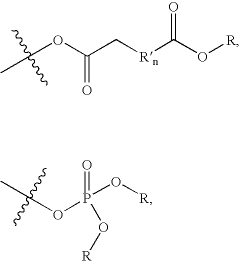
or a co-antioxidant. Each R′ may be CH₂. n may range from 1 to 9. Each R may be independently H, alkyl, aryl, benzyl, Group IA metal, or a co-antioxidant. Each co-antioxidant may be independently Vitamin C, Vitamin C analogs, Vitamin C derivatives, Vitamin E, Vitamin E analogs, Vitamin E derivatives, flavonoids, flavonoid analogs, or flavonoid derivatives. Flavonoids may include, for example, quercetin, xanthohumol, isoxanthohumol, or genistein.

Vitamin E may generally be divided into two categories including tocopherols having a general structure 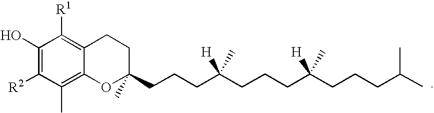
Alpha-tocopherol is used to designate when R¹═R²═CH₃. Beta-tocopherol is used to designate when R¹═CH₃ and R²═H. Gamma-tocopherol is used to designate when R¹═H and R²═CH₃. Delta-tocopherol is used to designate when R¹═R²═H.

The second category of Vitamin E may include tocotrienols having a general structure 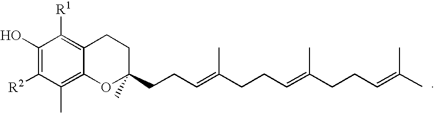
Alpha-tocotrienol is used to designate when R¹═R²═CH₃. Beta-tocotrienol is used to designate when R¹═CH₃ and R²═H. Gamma-tocotrienol is used to designate when R¹═H and R²═CH₃. Delta-tocotrienol is used to designate when R¹═R²═H.

Quercetin, a flavonoid, may have the structure

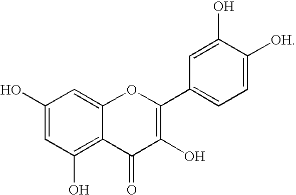

In some embodiments, the carotenoid analog or derivative may have the structure 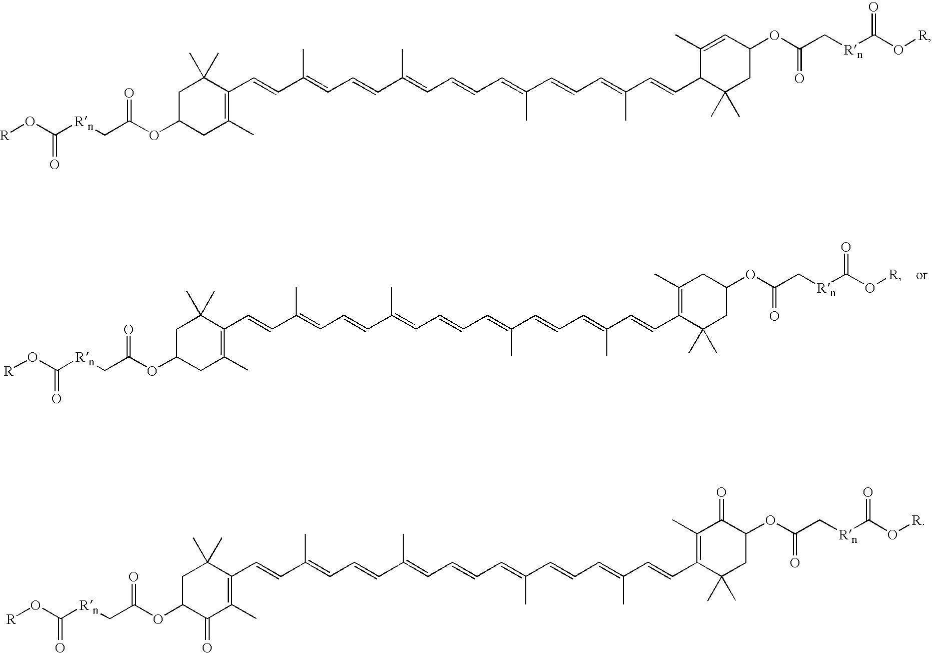
Each R may be independently H, alkyl, aryl, benzyl, Group IA metal, or a co-antioxidant. Each co-antioxidant may be independently Vitamin C, Vitamin C analogs, Vitamin C derivatives, Vitamin E, Vitamin E analogs, Vitamin E derivatives, flavonoids, flavonoid analogs, or flavonoid derivatives. Flavonoids may include, for example, quercetin, xanthohumol, isoxanthohumol, or genistein.

In some embodiments, the carotenoid analog or derivative may have the structure 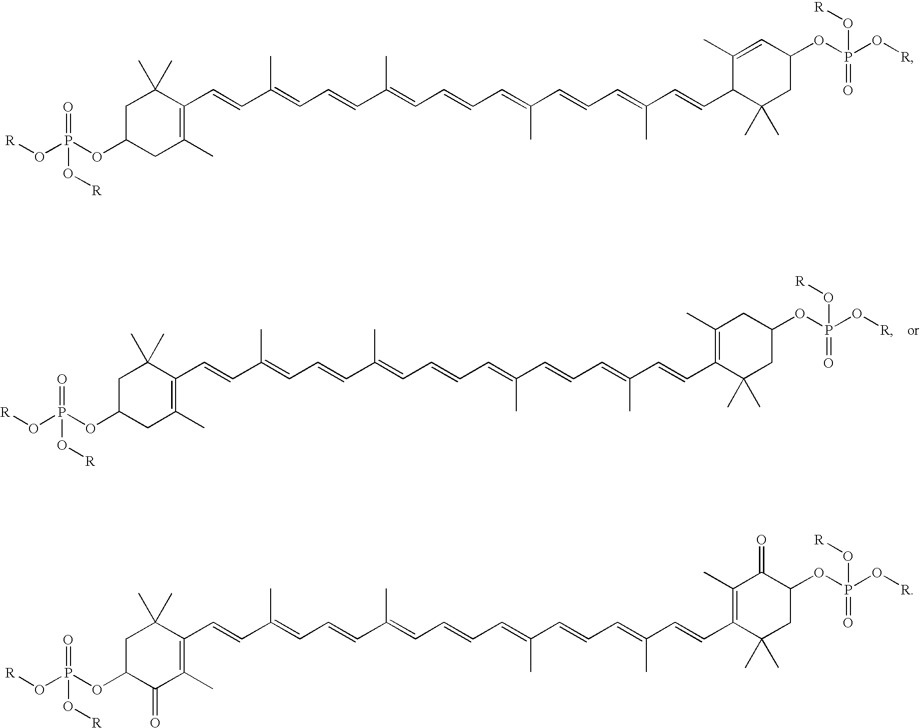
Each R may be independently H, alkyl, aryl, benzyl, Group IA metal (e.g., sodium), or a co-antioxidant. Each co-antioxidant may be independently Vitamin C, Vitamin C analogs, Vitamin C derivatives, Vitamin E, Vitamin E analogs, Vitamin E derivatives, flavonoids, flavonoid analogs, or flavonoid derivatives. Flavonoids may include, for example, quercetin, xanthohumol, isoxanthohumol, or genistein. When R includes Vitamin C, Vitamin C analogs, or Vitamin C derivatives, some embodiments may include carotenoid analogs or derivatives having the structure 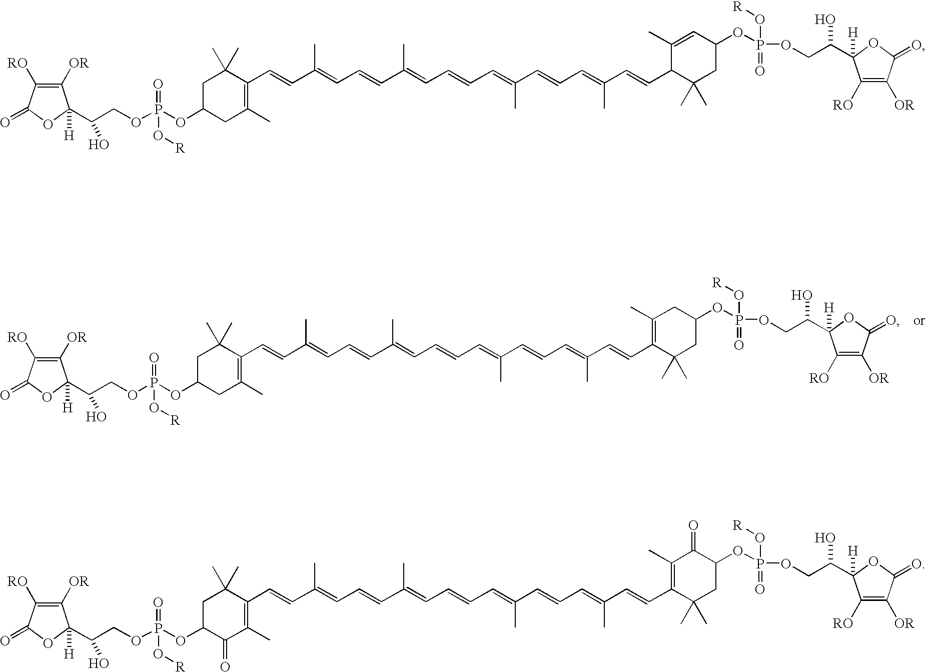
Each R may be independently H, alkyl, aryl, benzyl, or Group IA metal.

In some embodiments, a chemical compound including a carotenoid derivative may have the general structure (132): 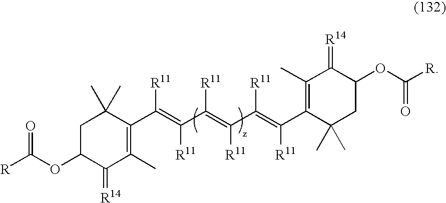
Each R¹¹ may be independently hydrogen or methyl. Each R¹⁴ may be independently O or H₂. Each R may be independently OR¹² or R¹². Each R¹² may be independently -alkyl-NR¹³₃⁺, -aromatic-NR¹³₃⁺, -alkyl-CO₂⁻, -aromatic-CO₂⁻, -amino acid-NH₃⁺, -phosphorylated amino acid-NH₃⁺, polyethylene glycol, dextran, H, alkyl, co-antioxidant (e.g. Vitamin C, Vitamin C analogs, Vitamin C derivatives, Vitamin E, Vitamin E analogs, Vitamin E derivatives, flavonoids, flavonoid analogs, or flavonoid derivatives), or aryl. Each R¹³ may be independently H, alkyl, or aryl. z may range from 5 to 12. In some embodiments, z may range from about 3 to about 15. In certain embodiments, the maximum value of z may only be limited by the ultimate size of the chemical compound, particularly as it relates to the size of the chemical compound and the potential interference with the chemical compound's biological availability as discussed herein. In some embodiments, substituents may be at least partially hydrophilic. These carotenoid derivatives may be used in a pharmaceutical composition.

In some embodiments, a chemical compound including a carotenoid derivative may have the general structure (134): 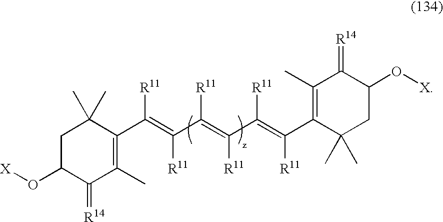
Each R¹¹ may be independently hydrogen or methyl. Each R¹⁴ may be independently O or H₂. Each X may be independently 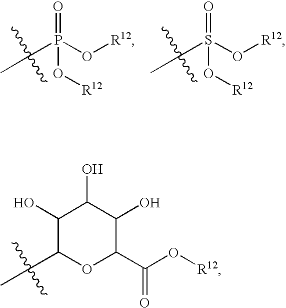
-alkyl-NR¹²₃⁺, -aromatic-NR¹²₃⁺, -alkyl-CO₂⁻, -aromatic-CO₂⁻, -amino acid-NH₃⁺, -phosphorylated amino acid-NH₃⁺, polyethylene glycol, dextran, alkyl, Group IA metal, co-antioxidant (e.g. Vitamin C, Vitamin C analogs, Vitamin C derivatives, Vitamin E, Vitamin E analogs, Vitamin E derivatives, flavonoids, flavonoid analogs, or flavonoid derivatives), or aryl. Each R¹² is independently -alkyl-N R¹³₃⁺, -aromatic-NR¹³₃⁺, -alkyl-CO₂⁻, -aromatic-CO₂⁻, -amino acid-NH₃⁺, -phosphorylated amino acid-NH₃⁺, polyethylene glycol, dextran, H, alkyl, aryl, benzyl, Group IA metal, co-antioxidant (e.g. Vitamin C, Vitamin C analogs, Vitamin C derivatives, Vitamin E, Vitamin E analogs, Vitamin E derivatives, flavonoids, flavonoid analogs, or flavonoid derivatives), or Group IA salt. Each R¹³ may be independently H, alkyl, or aryl. z may range from 5 to 12. In some embodiments, z may range from about 3 to about 15. In certain embodiments, the maximum value of z may only be limited by the ultimate size of the chemical compound, particularly as it relates to the size of the chemical compound and the potential interference with the chemical compound's biological availability as discussed herein. In some embodiments, substituents may be at least partially hydrophilic. These carotenoid derivatives may be used in a pharmaceutical composition.

In some non-limiting examples, five- and/or six-membered ring carotenoid derivatives may be more easily synthesized. Synthesis may come more easily due to, for example, the natural stability of five- and six-membered rings. Synthesis of carotenoid derivatives including five- and/or six-membered rings may be more easily synthesized due to, for example, the availability of naturally occurring carotenoids including five- and/or six-membered rings. In some embodiments, five-membered rings may decrease steric hindrance associated with rotation of the cyclic ring around the molecular bond connecting the cyclic ring to the polyene chain. Reducing steric hindrance may allow greater overlap of any π oribitals within a cyclic ring with the polyene chain, thereby increasing the degree of conjugation and effective chromophore length of the molecule. This may have the salutatory effect of increasing antioxidant capacity of the carotenoid derivatives.

In some embodiments, a substituent (W) may be at least partially hydrophilic. A hydrophilic substituent may assist in increasing the water solubility of a carotenoid derivative. In some embodiments, a carotenoid derivative may be at least partially water-soluble. The cyclic ring may include at least one chiral center. The acyclic alkene may include at least one chiral center. The cyclic ring may include at least one degree of unsaturation. In some cyclic ring embodiments, the cyclic ring may be aromatic. One or more degrees of unsaturation within the ring may assist in extending the conjugation of the carotenoid derivative. Extending conjugation within the carotenoid derivative may have the salutatory effect of increasing the antioxidant properties of the carotenoid derivatives. In some embodiments, the substituent W may include, for example, a carboxylic acid, an amino acid, an ester, an alkanol, an amine, a phosphate, a succinate, a glycinate, an ether, a glucoside, a sugar, or a carboxylate salt.

In some embodiments, each substituent —W may independently include —XR. Each X may independently include O, N, or S. In some embodiments, each substituent —W may independently comprise amino acids, esters, carbamates, amides, carbonates, alcohol, phosphates, or sulfonates. In some substituent embodiments, the substituent may include, for example (d) through (uu): 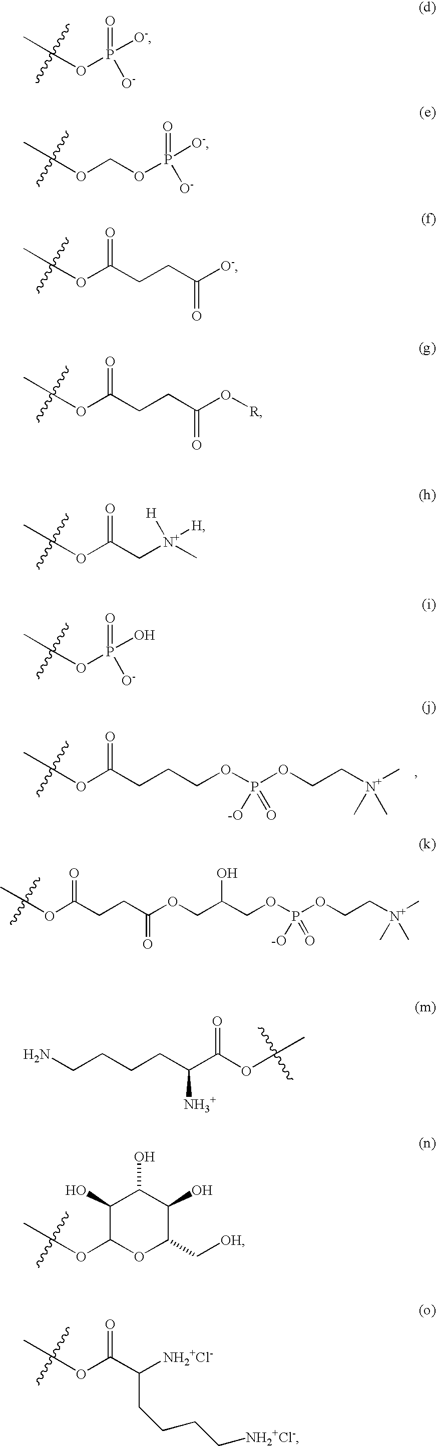 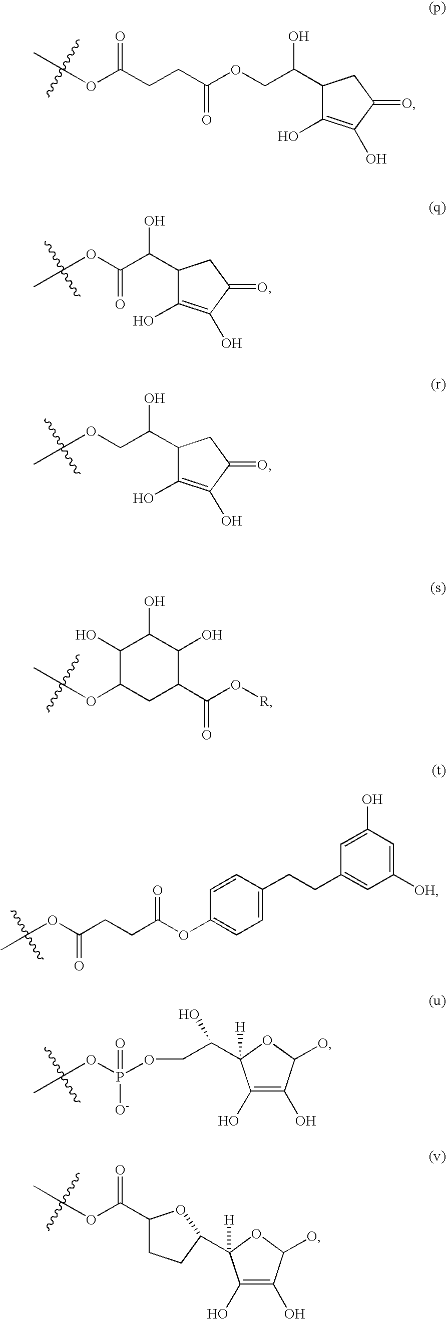 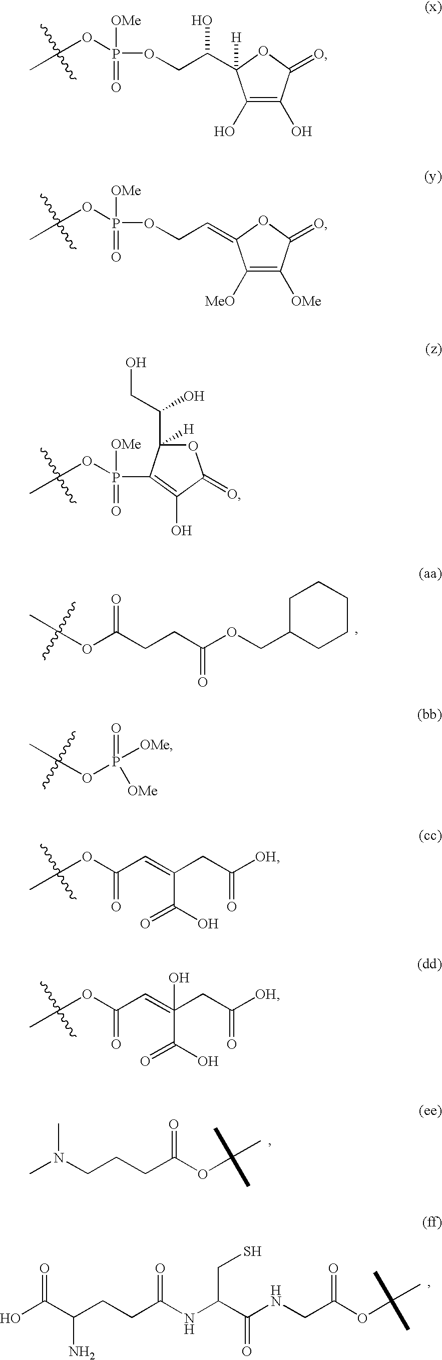 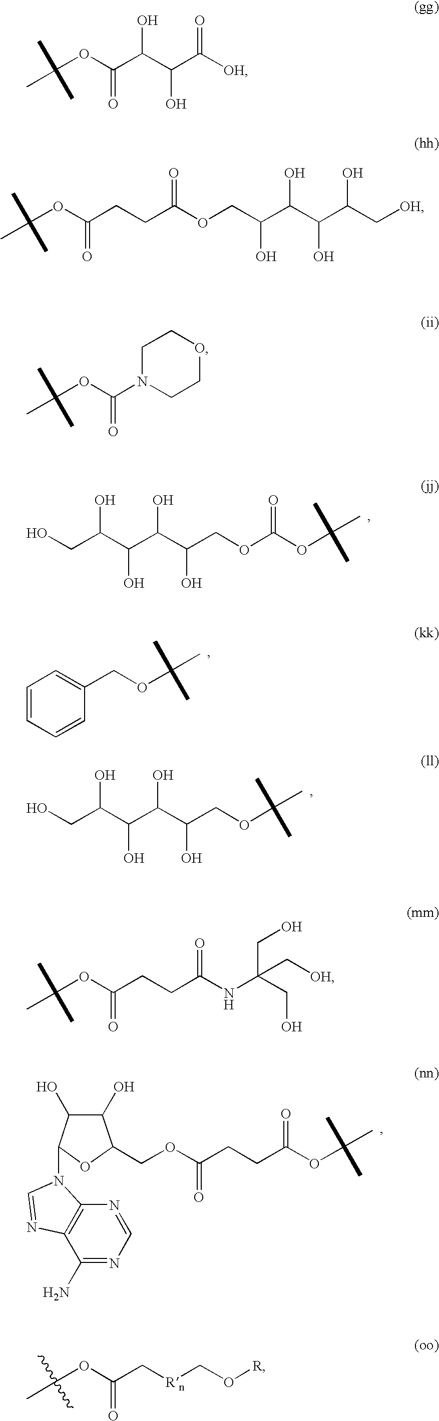 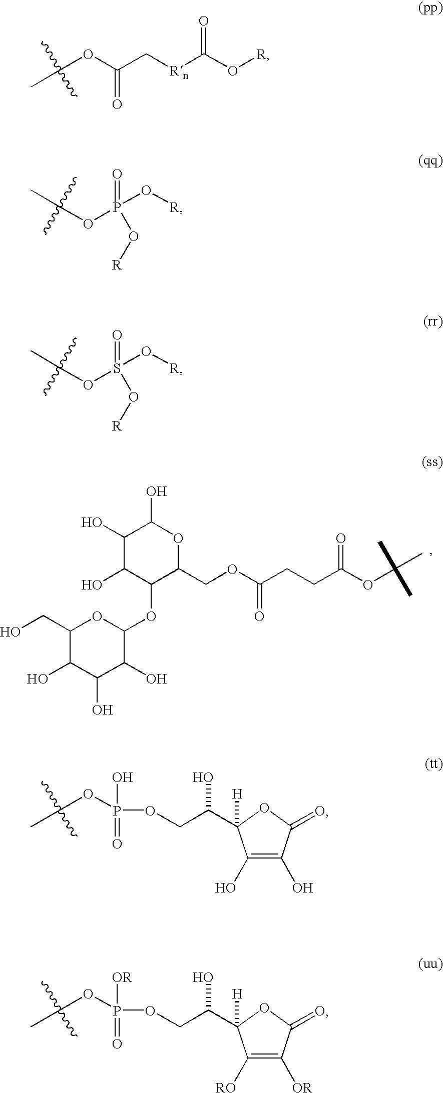
where each R is, for example, independently -alkyl-NR¹²₃⁺, -aromatic-NR¹²₃⁺, -alkyl-CO₂⁻, -aromatic-CO₂⁻, -amino acid-NH₃⁺, -phosphorylated amino acid-NH₃⁺, polyethylene glycol, dextran, H, alkyl, Group IA metal, co-antioxidant (e.g. Vitamin C, Vitamin C analogs, Vitamin C derivatives, Vitamin E, Vitamin E analogs, Vitamin E derivatives, flavonoids, flavonoid analogs, or flavonoid derivatives), or aryl. Each R′ may be CH₂. n may range from 1 to 9. In some embodiments, substituents may include any combination of (d) through (uu). In some embodiments, negatively charged substituents may include Group IA metals, one metal or a combination of different Group IA metals in an embodiment with more than one negatively charged substituent, as counter ions. Group IA metals may include, but are not limited to, sodium, potassium, and/or lithium.

Water-soluble carotenoid analogs or derivatives may have a water solubility of greater than about 1 mg/mL in some embodiments. In certain embodiments, water-soluble carotenoid analogs or derivatives may have a water solubility of greater than about 5 mg/mL. In certain embodiments, water-soluble carotenoid analogs or derivatives may have a water solubility of greater than about 10 mg/mL. In certain embodiments, water-soluble carotenoid analogs or derivatives may have a water solubility of greater than about 20 mg/mL. In some embodiments, water-soluble carotenoid analogs or derivatives may have a water solubility of greater than about 50 mg/mL.

Naturally occurring carotenoids such as xanthophyll carotenoids of the C40 series, which includes commercially important compounds such as lutein, zeaxanthin, and astaxanthin, have poor aqueous solubility in the native state. Varying the chemical structure(s) of the esterified moieties may vastly increase the aqueous solubility and/or dispersibility of derivatized carotenoids.

In some embodiments, highly water-dispersible C40 carotenoid derivatives may include natural source RRR-lutein (β,ε-carotene-3,3′-diol) derivatives. Derivatives may be synthesized by esterification with inorganic phosphate and succinic acid, respectively, and subsequently converted to the sodium salts. Deep orange, evenly colored aqueous suspensions were obtained after addition of these derivatives to USP-purified water. Aqueous dispersibility of the disuccinate sodium salt of natural lutein was 2.85 mg/mL; the diphosphate salt demonstrated a >10-fold increase in dispersibility at 29.27 mg/mL. Aqueous suspensions may be obtained without the addition of heat, detergents, co-solvents, or other additives.

The direct aqueous superoxide scavenging abilities of these derivatives were subsequently evaluated by electron paramagnetic resonance (EPR) spectroscopy in a well-characterized in vitro isolated human neutrophil assay. The derivatives may be potent (millimolar concentration) and nearly identical aqueous-phase scavengers, demonstrating dose-dependent suppression of the superoxide anion signal (as detected by spin-trap adducts of DEPMPO) in the millimolar range. Evidence of card-pack aggregation was obtained for the diphosphate derivative with UV-V is spectroscopy (discussed herein), whereas limited card-pack and/or head-to-tail aggregation was noted for the disuccinate derivative. These lutein-based soft drugs may find utility in those commercial and clinical applications for which aqueous-phase singlet oxygen quenching and direct radical scavenging may be required.

The absolute size of a carotenoid derivative (in 3 dimensions) is important when considering its use in biological and/or medicinal applications. Some of the largest naturally occurring carotenoids are no greater than about C₅₀. This is probably due to size limits imposed on molecules requiring incorporation into and/or interaction with cellular membranes. Cellular membranes may be particularly co-evolved with molecules of a length of approximately 30 nm. In some embodiments, carotenoid derivatives may be greater than or less than about 30 nm in size. In certain embodiments, carotenoid derivatives may be able to change conformation and/or otherwise assume an appropriate shape, which effectively enables the carotenoid derivative to efficiently interact with a cellular membrane.

Although the above structure, and subsequent structures, depict alkenes in the E configuration this should not be seen as limiting. Compounds discussed herein may include embodiments where alkenes are in the Z configuration or include alkenes in a combination of Z and E configurations within the same molecule. The compounds depicted herein may naturally convert between the Z and E configuration and/or exist in equilibrium between the two configurations.

In an embodiment, a chemical compound may include a carotenoid derivative having the structure (136) 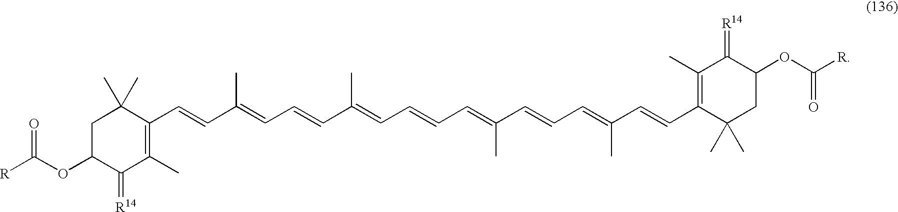
Each R¹⁴ may be independently O or H₂. Each R may be independently OR¹² or R¹². Each R¹² may be independently -alkyl-NR¹³₃⁺, -aromatic-NR¹³₃⁺, -alkyl-CO₂⁻, -aromatic-CO₂⁻, -amino acid-NH₃⁺, phosphorylated amino acid-NH₃⁺, polyethylene glycol, dextran, H, alkyl, peptides, poly-lysine, co-antioxidant (e.g. Vitamin C, Vitamin C analogs, Vitamin C derivatives, Vitamin E, Vitamin E analogs, Vitamin E derivatives, flavonoids, flavonoid analogs, or flavonoid derivatives), or aryl. In addition, each R¹³ may be independently H, alkyl, or aryl. The carotenoid derivative may include at least one chiral center.

In a specific embodiment where R¹⁴ is H₂, the carotenoid derivative may have the structure (138)

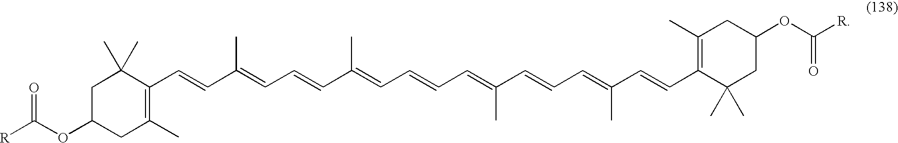

In a specific embodiment where R¹⁴ is O, the carotenoid derivative may have the structure (140)

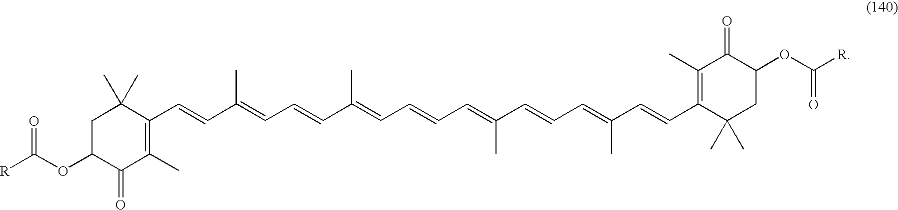

In an embodiment, a chemical compound may include a carotenoid derivative having the structure (142) 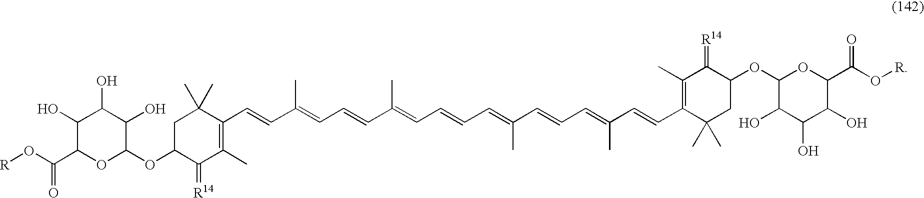
Each R¹⁴ may be independently O or H₂. Each R may be independently H, alkyl, benzyl, Group IA metal, co-antioxidant, or aryl. The carotenoid derivative may include at least one chiral center. In a specific embodiment R¹⁴ may be H₂, the carotenoid derivative having the structure (144)

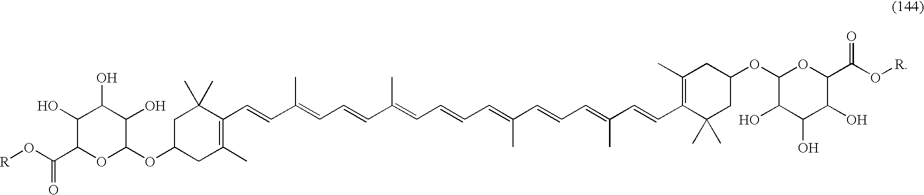

In a specific embodiment where R¹⁴ is O, the carotenoid derivative may have the structure (146)

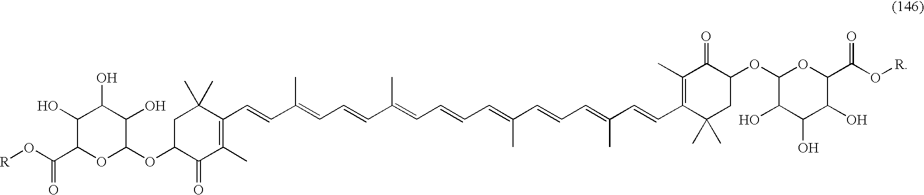

In an embodiment, a chemical compound may include a carotenoid derivative having the structure (148) 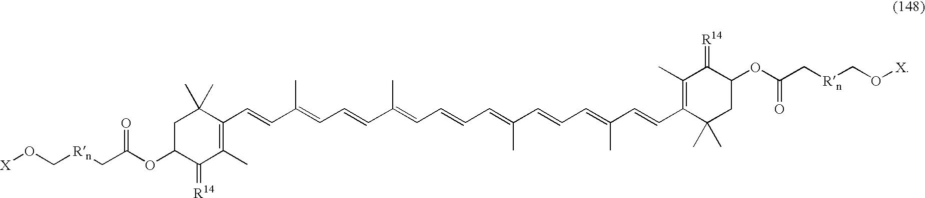
Each R¹⁴ may be independently O or H₂. Each R′ may be CH₂. n may range from 1 to 9. Each X may be independently 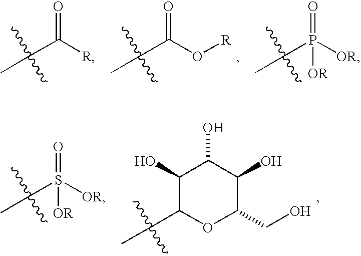
Group IA metal, or co-antioxidant (e.g. Vitamin C, Vitamin C analogs, Vitamin C derivatives, Vitamin E, Vitamin E analogs, Vitamin E derivatives, flavonoids, flavonoid analogs, or flavonoid derivatives). Each R may be independently -alkyl-NR¹²₃⁺, -aromatic-NR¹²₃⁺, -alkyl-CO₂⁻, -aromatic-CO₂⁻, -amino acid-NH₃⁺, -phosphorylated amino acid-NH₃⁺, polyethylene glycol, dextran, H, alkyl, Group IA metal, benzyl, co-antioxidant (e.g. Vitamin C, Vitamin C analogs, Vitamin C derivatives, Vitamin E, Vitamin E analogs, Vitamin E derivatives, flavonoids, flavonoid analogs, or flavonoid derivatives), or aryl. Each R¹² may be independently H, alkyl, or aryl. The carotenoid derivative may include at least one chiral center.

In a specific embodiment where R¹⁴ is H₂, the carotenoid derivative may have the structure (150) 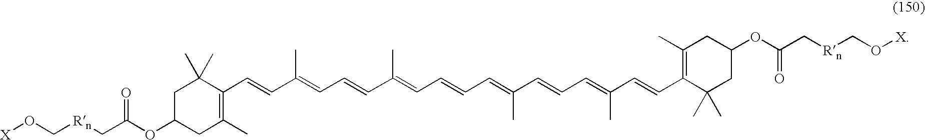
In a specific embodiment where R¹⁴ is O, the carotenoid derivative may have the structure (152)

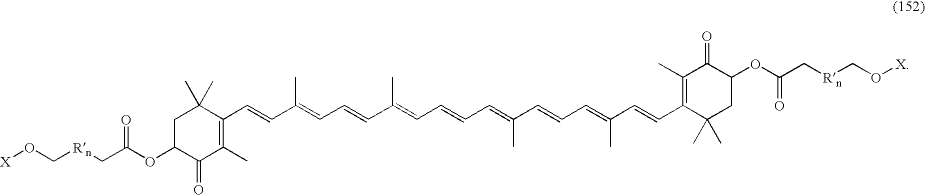

In an embodiment, a chemical compound may include a carotenoid derivative having the structure (148) 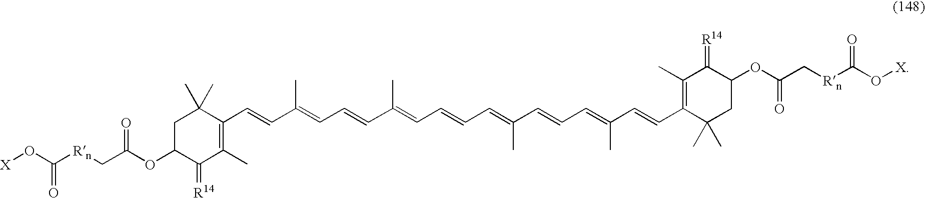
Each R¹⁴ may be independently O or H₂. Each R′ may be CH₂. n may range from 1 to 9. Each X may be independently 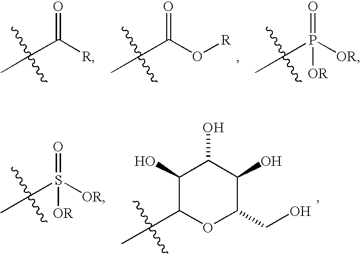
Group IA metal, or co-antioxidant (e.g. Vitamin C, Vitamin C analogs, Vitamin C derivatives, Vitamin E, Vitamin E analogs, Vitamin E derivatives, flavonoids, flavonoid analogs, or flavonoid derivatives). Each R may be independently -alkyl-NR¹²₃⁺, -aromatic-NR¹²₃⁺, -alkyl-CO₂⁻, -aromatic-CO₂⁻, -amino acid-NH₃⁺, -phosphorylated amino acid-NH₃⁺, polyethylene glycol, dextran, H, alkyl, Group IA metal, co-antioxidant (e.g. Vitamin C, Vitamin C analogs, Vitamin C derivatives, Vitamin E, Vitamin E analogs, Vitamin E derivatives, flavonoids, flavonoid analogs, or flavonoid derivatives), or aryl. Each R¹² may be independently H, alkyl, or aryl. The carotenoid derivative may include at least one chiral center.

In a specific embodiment where R¹⁴ is H₂, the carotenoid derivative may have the structure (150) 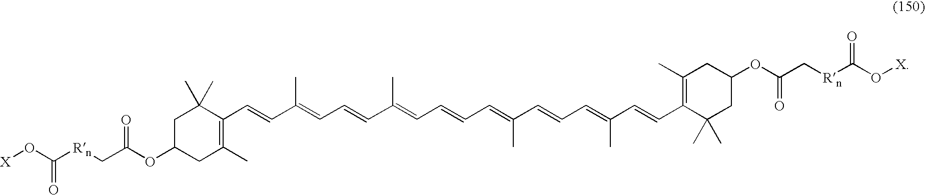
In a specific embodiment where R¹⁴ is O, the carotenoid derivative may have the structure (152)

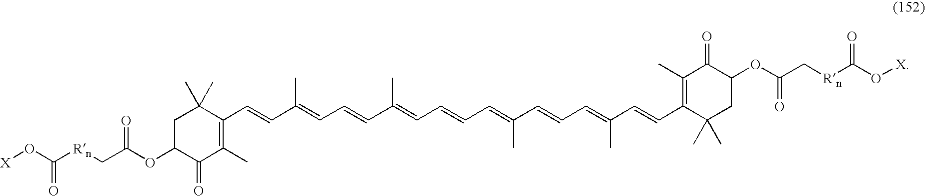

In an embodiment, a chemical compound may include a carotenoid derivative having the structure (154) 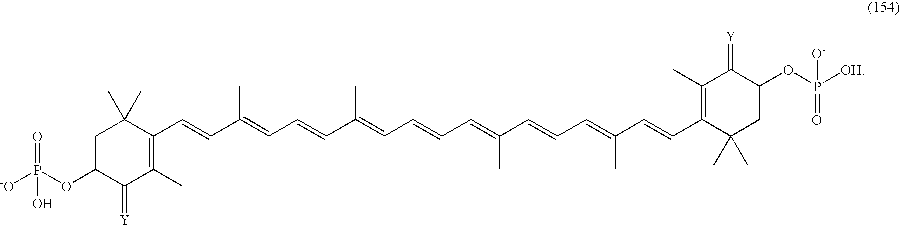
Each R¹⁴ may be independently O or H₂. The carotenoid derivative may include at least one chiral center. In a specific embodiment R¹⁴ may be H₂, the carotenoid derivative having the structure (156) 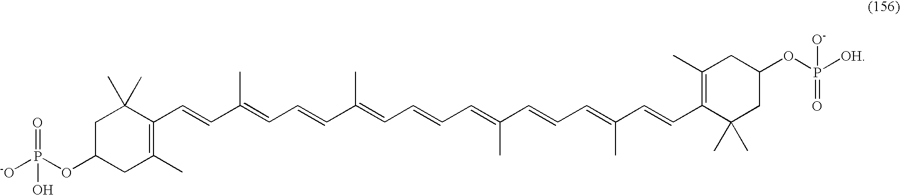
In a specific embodiment where R¹⁴ is O, the carotenoid derivative may have the structure (158) 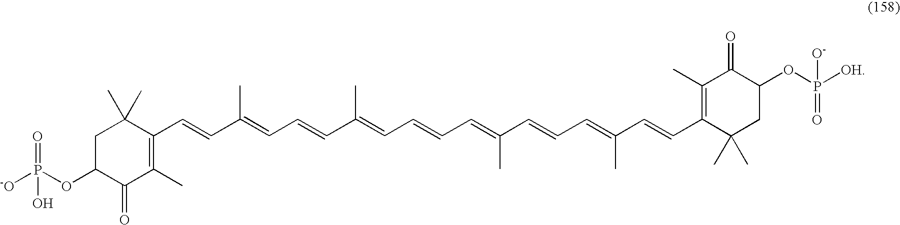
In some embodiments, a chemical compound may include a disuccinic acid ester carotenoid derivative having the structure (160)

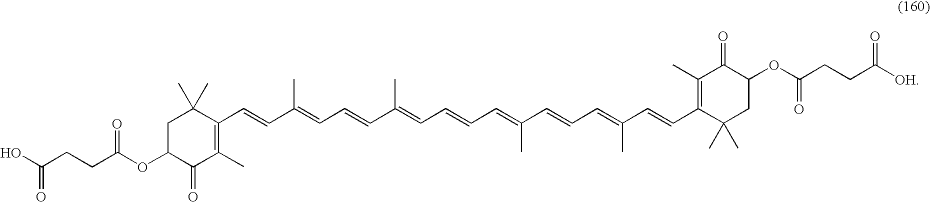

In some embodiments, a chemical compound may include a disodium salt disuccinic acid ester carotenoid derivative having the structure (162)

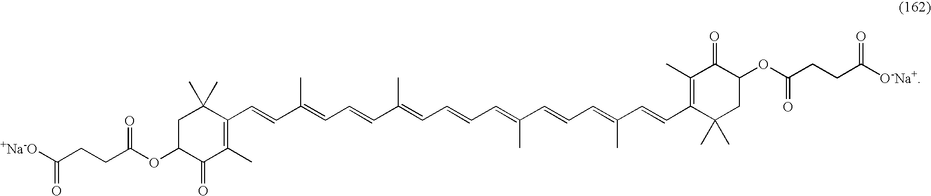

In some embodiments, a chemical compound may include a carotenoid derivative with a co-antioxidant, in particular one or more analogs or derivatives of vitamin C (i.e., L ascorbic acid) coupled to a carotenoid. Some embodiments may include carboxylic acid and/or carboxylate derivatives of vitamin C coupled to a carotenoid (e.g., structure (164)) 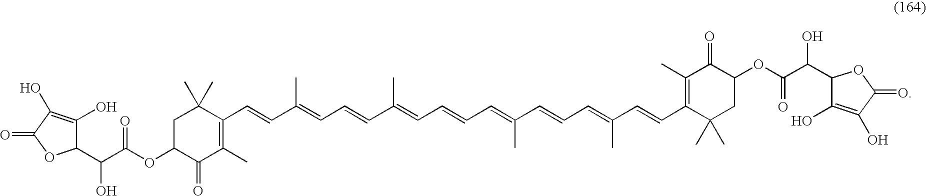
Carbohydr. Res. 1978, 60, 251-258, herein incorporated by reference, discloses oxidation at C-6 of ascorbic acid as depicted in EQN. 5. 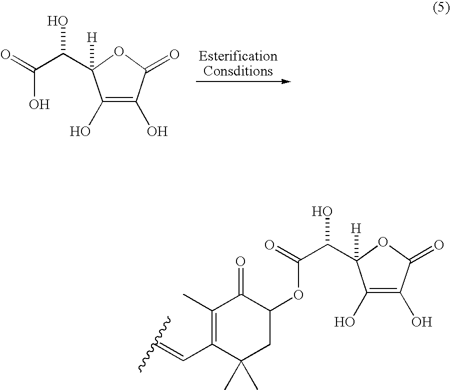
Some embodiments may include vitamin C and/or vitamin C analogs or derivatives coupled to a carotenoid. Vitamin C may be coupled to the carotenoid via an ether linkage (e.g., structure (166)) 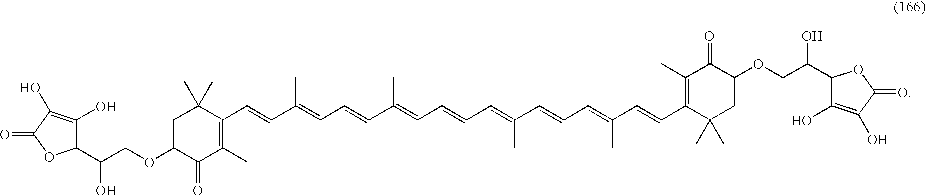
Some embodiments may include vitamin C disuccinate analogs or derivatives coupled to a carotenoid (e.g., structure (168)) 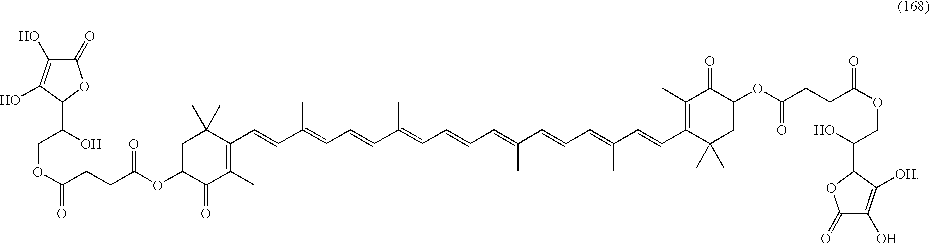
Some embodiments may include solutions or pharmaceutical preparations of carotenoids and/or carotenoid derivatives combined with co-antioxidants, in particular vitamin C and/or vitamin C analogs or derivatives. Pharmaceutical preparations may include about a 2:1 ratio of vitamin C to carotenoid respectively.

In some embodiments, co-antioxidants (e.g., vitamin C) may increase solubility of the chemical compound. In certain embodiments, co-antioxidants (e.g., vitamin C) may decrease toxicity associated with at least some carotenoid analogs or derivatives. In certain embodiments, co-antioxidants (e.g., vitamin C) may increase the potency of the chemical compound synergistically. Co-antioxidants may be coupled (e.g., a covalent bond) to the carotenoid derivative. Co-antioxidants may be included as a part of a pharmaceutically acceptable formulation.

In some embodiments, a carotenoid (e.g., astaxanthin) may be coupled to vitamin C forming an ether linkage. The ether linkage may be formed using the Mitsunobu reaction as in EQN. 1.

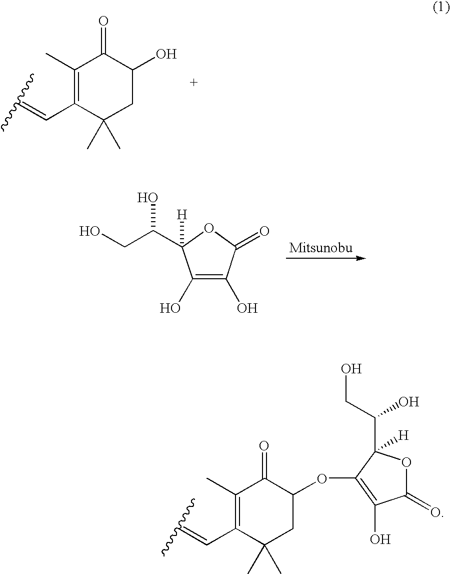

In some embodiments, vitamin C may be selectively esterified. Vitamin C may be selectively esterified at the C-3 position (e.g., EQN. 2). J. Org. Chem. 2000, 65, 911-913, herein incorporated by reference, discloses selective esterification at C-3 of unprotected ascorbic acid with primary alcohols.

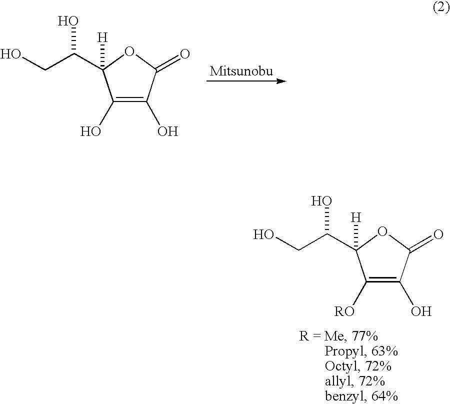

In some embodiments, a carotenoid may be coupled to vitamin C. Vitamin C may be coupled to the carotenoid at the C-6, C-5 diol position as depicted in EQNS. 3 and 4 forming an acetal.

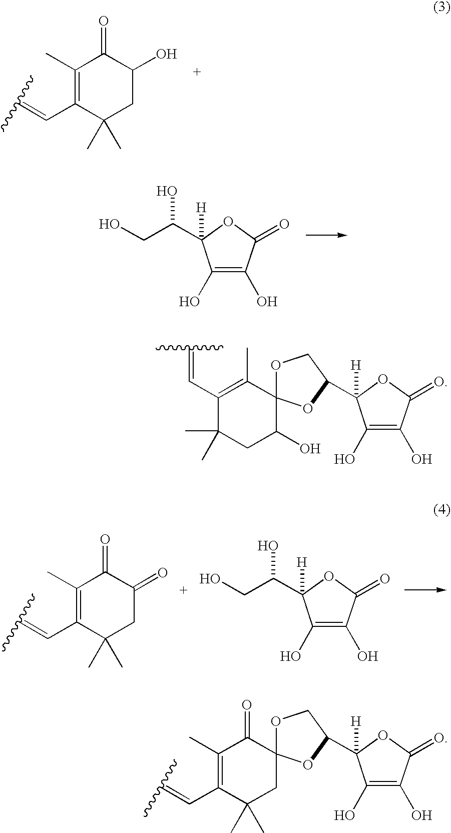

In some embodiments, a carotenoid may be coupled to a water-soluble moiety (e.g., vitamin C) with a glyoxylate linker as depicted in EQN. 6. Tetrahedron 1989, 22, 6987-6998, herein incorporated by reference, discloses similar acetal formations.

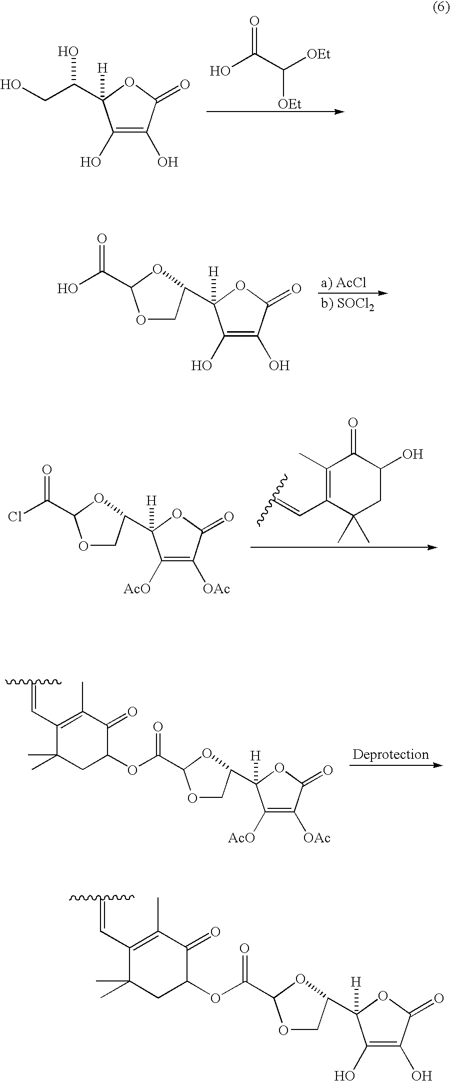

In some embodiments, a carotenoid may be coupled to a water-soluble moiety (e.g., vitamin C) with a glyoxylate linker as depicted in EQN. 7. J. Med. Chem. 1988, 31, 1363-1368, herein incorporated by reference, discloses the glyoxylic acid chloride.

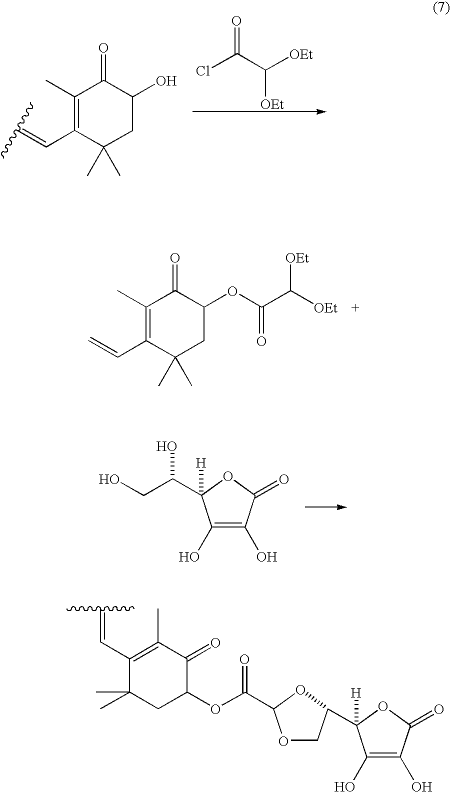

In some embodiments, a carotenoid may be coupled to a water-soluble moiety (e.g., vitamin C) with a phosphate linker as depicted in EQN. 8. Carbohydr. Res. 1988, 176, 73-78, herein incorporated by reference, discloses the L-ascorbate 6-phosphate.

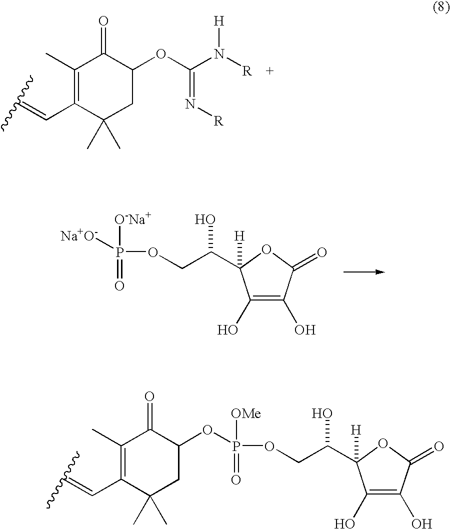

In some embodiments, a carotenoid may be coupled to a water-soluble moiety (e.g., vitamin C) with a phosphate linker as depicted in EQN. 9. Carbohydr. Res. 1979, 68, 313-319, herein incorporated by reference, discloses the 6-bromo derivative of vitamin C. Carbohydr. Res. 1988, 176, 73-78, herein incorporated by reference, discloses the 6-bromo derivative of vitamin C's reaction with phosphates.

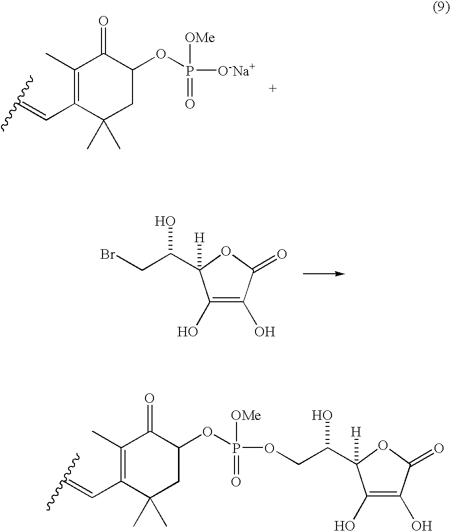

In some embodiments, a carotenoid may be coupled to a water-soluble moiety (e.g., vitamin C) with a phosphate linker as depicted in EQN. 10. J. Med Chem. 2001, 44, 1749-1757 and J. Med Chem. 2001, 44, 3710-3720, herein incorporated by reference, disclose the allyl chloride derivative and its reaction with nucleophiles, including phosphates, under mild basic conditions.

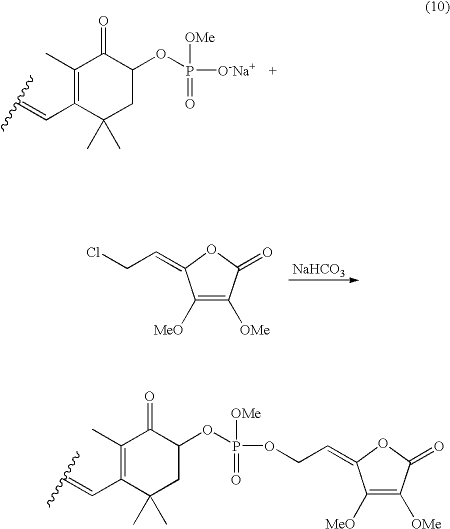

In some embodiments, a carotenoid may be coupled to a water-soluble moiety (e.g., vitamin C) with a phosphate linker as depicted in EQN. 11. Vitamin C may be coupled to the carotenoid using selective esterification at C-3 of unprotected ascorbic acid with primary alcohols.

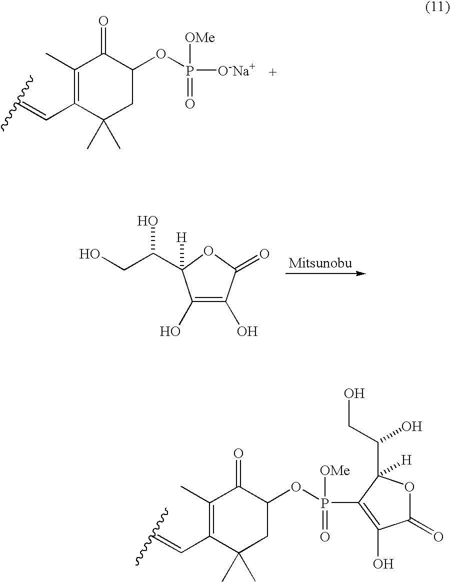

In some embodiments, a carotenoid may be coupled to a water-soluble moiety (e.g., vitamin C) with a phosphate linker as in 242. Structure 242 may include one or more counterions (e.g., Group IA metals). 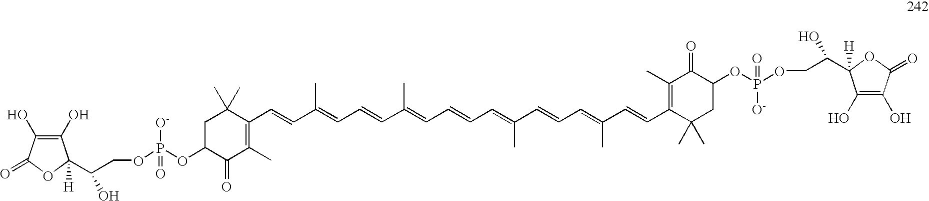
EQN. 12 depicts an example of a synthesis of a protected form of 242.

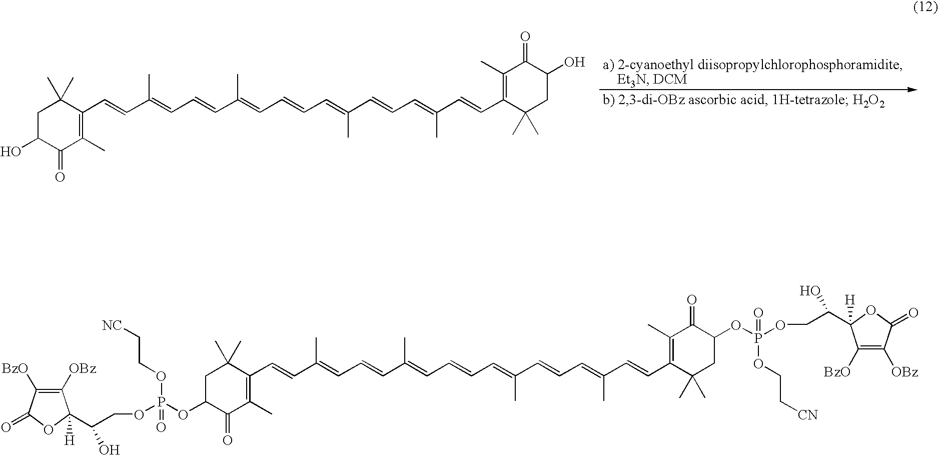

In some embodiments, a chemical compound may include a carotenoid derivative including one or more amino acids (e.g., lysine) and/or amino acid analogs or derivatives (e.g., lysine hydrochloric acid salt) coupled to a carotenoid (e.g., structure (170)).

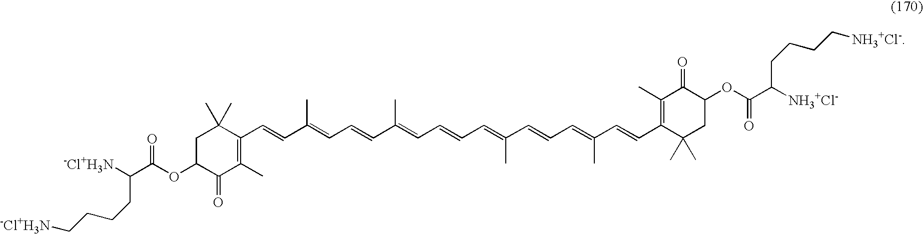

In some embodiments, a carotenoid analog or derivative may include:

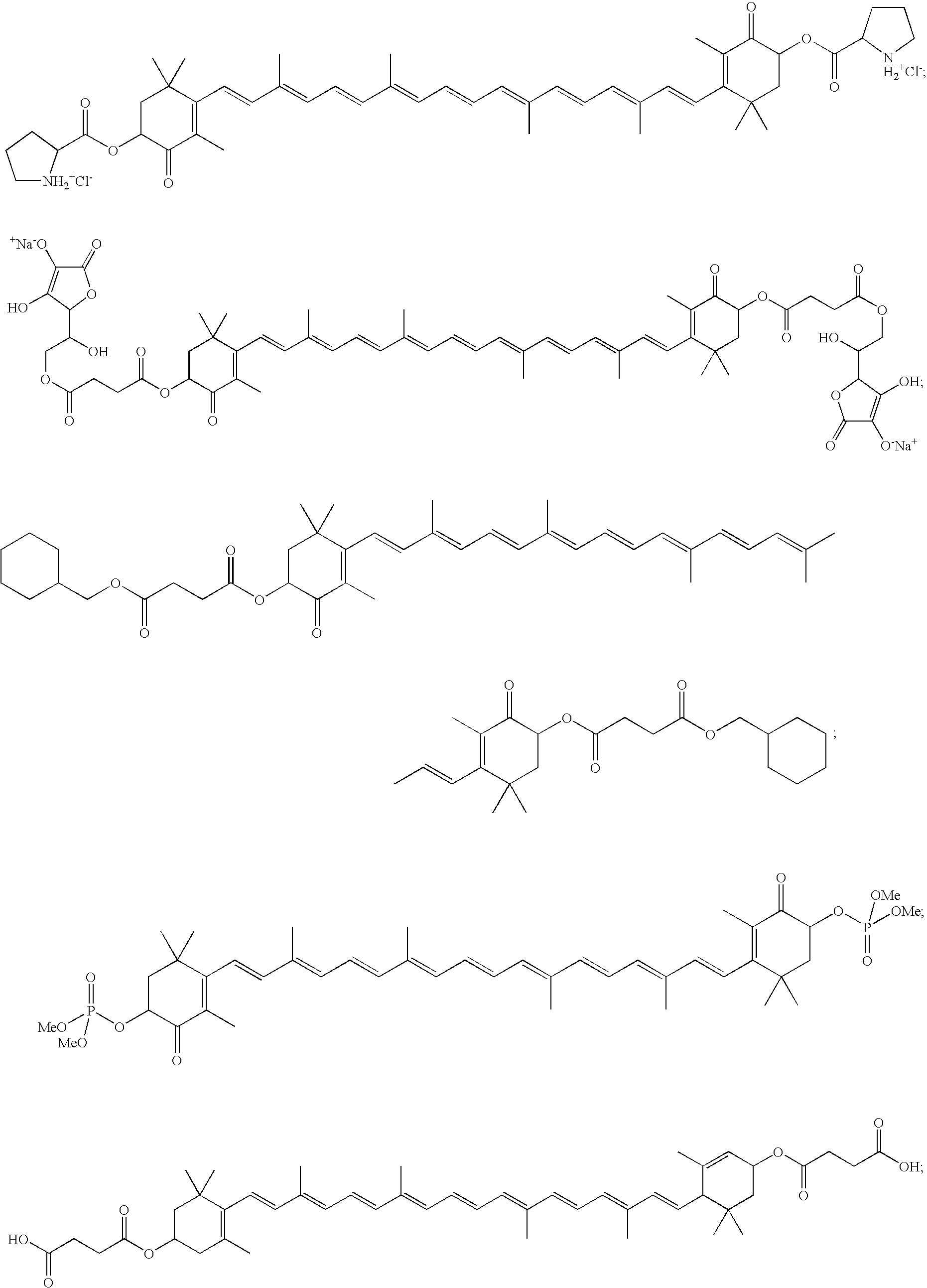

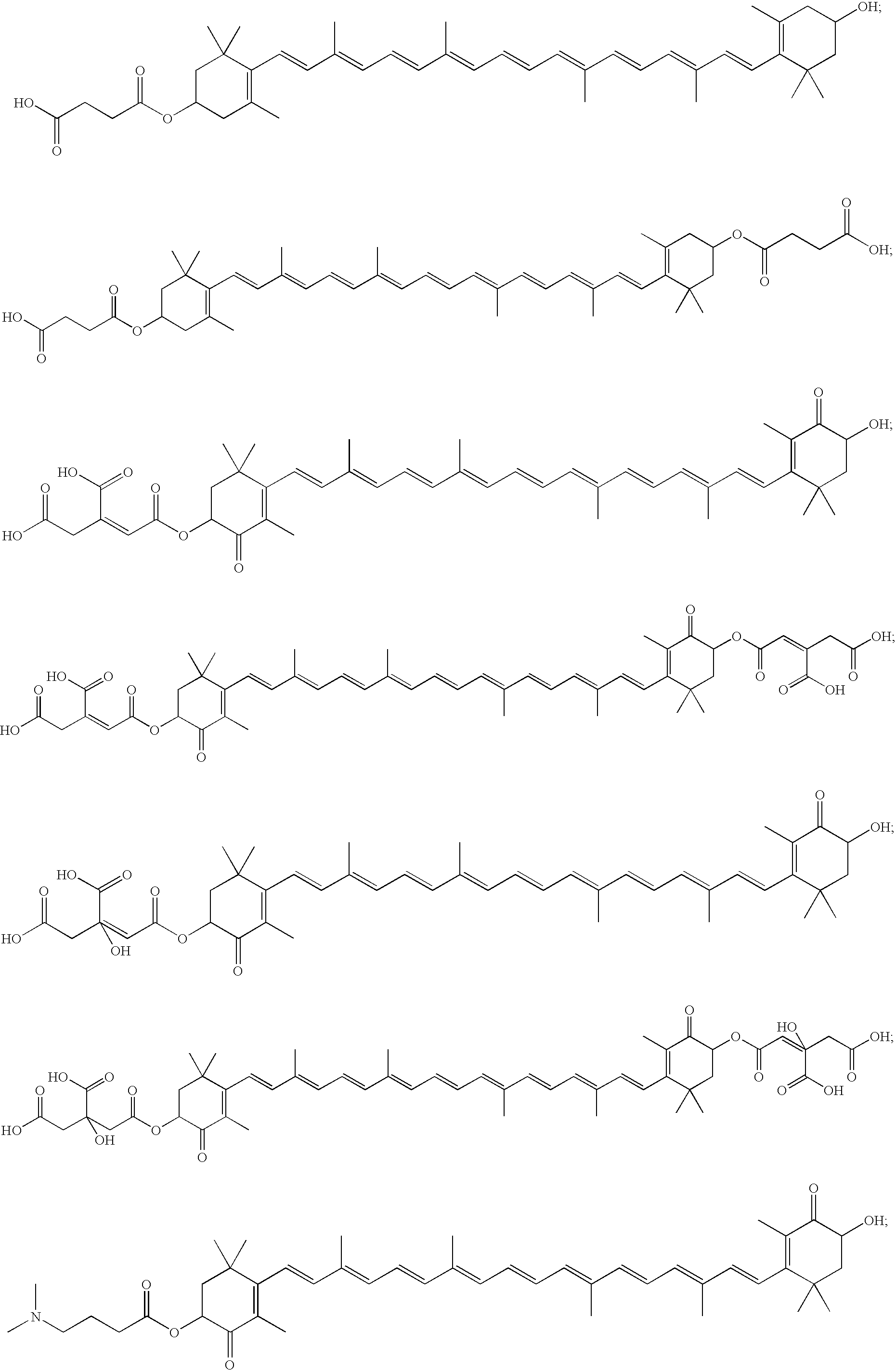

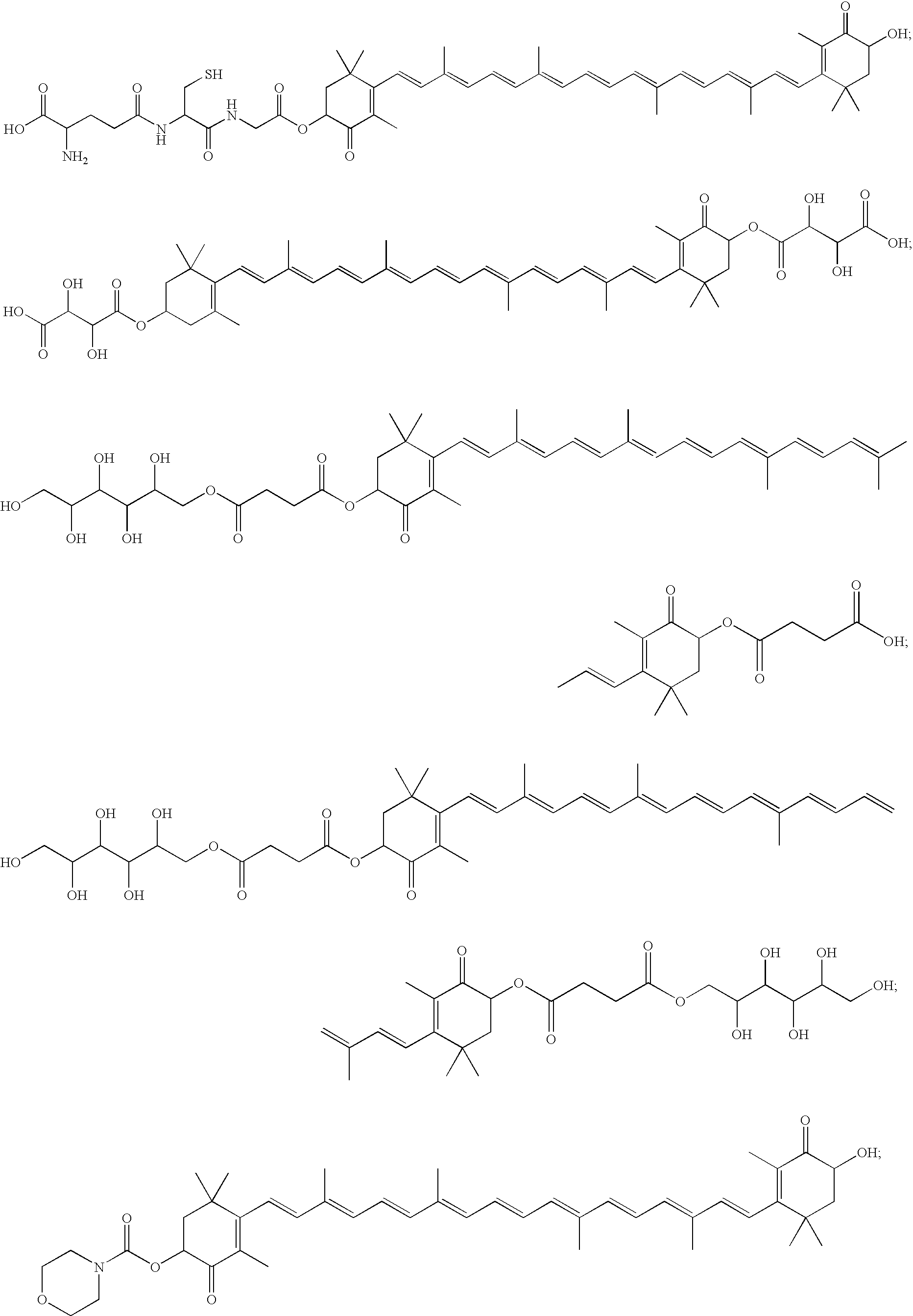

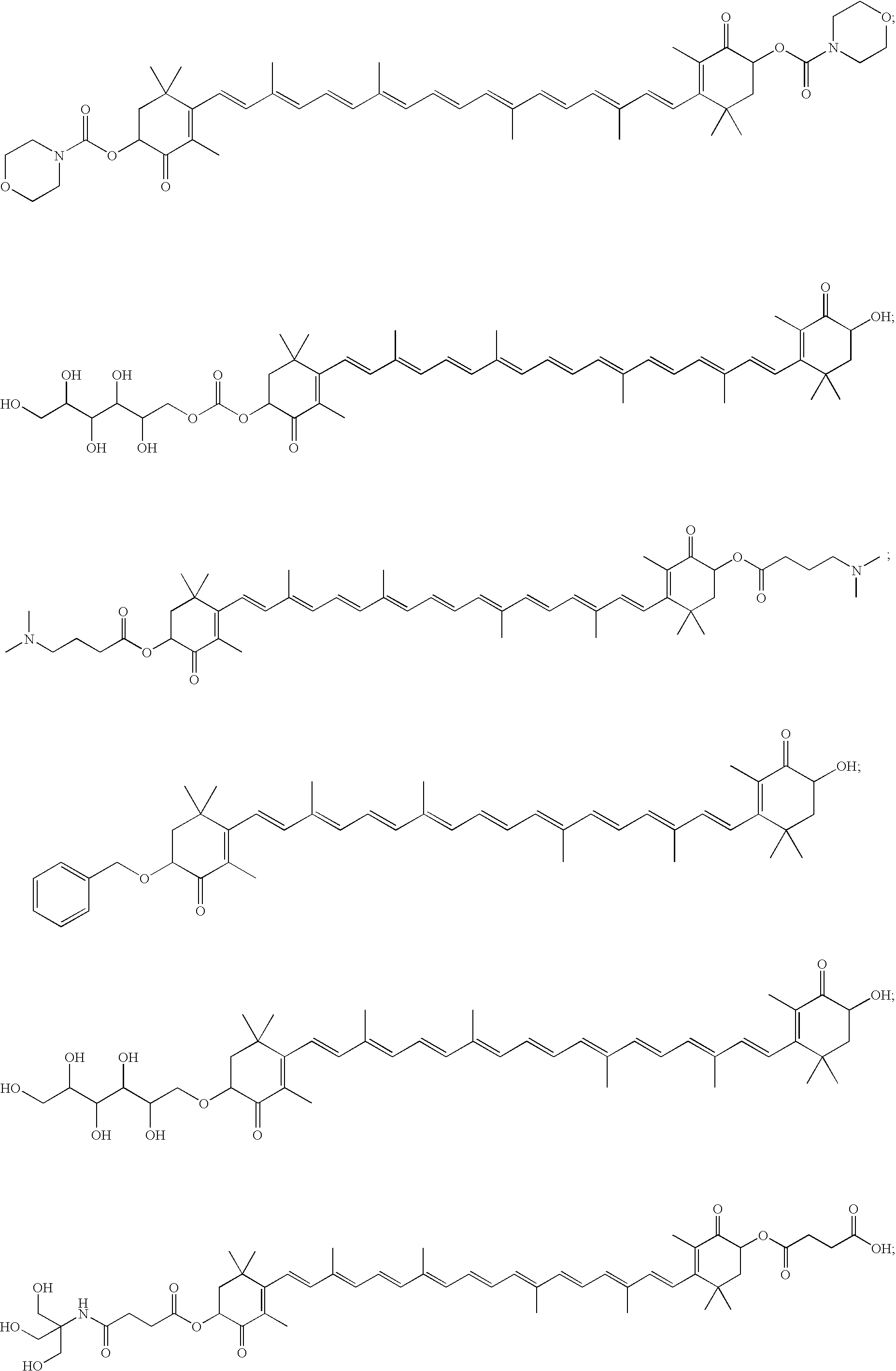

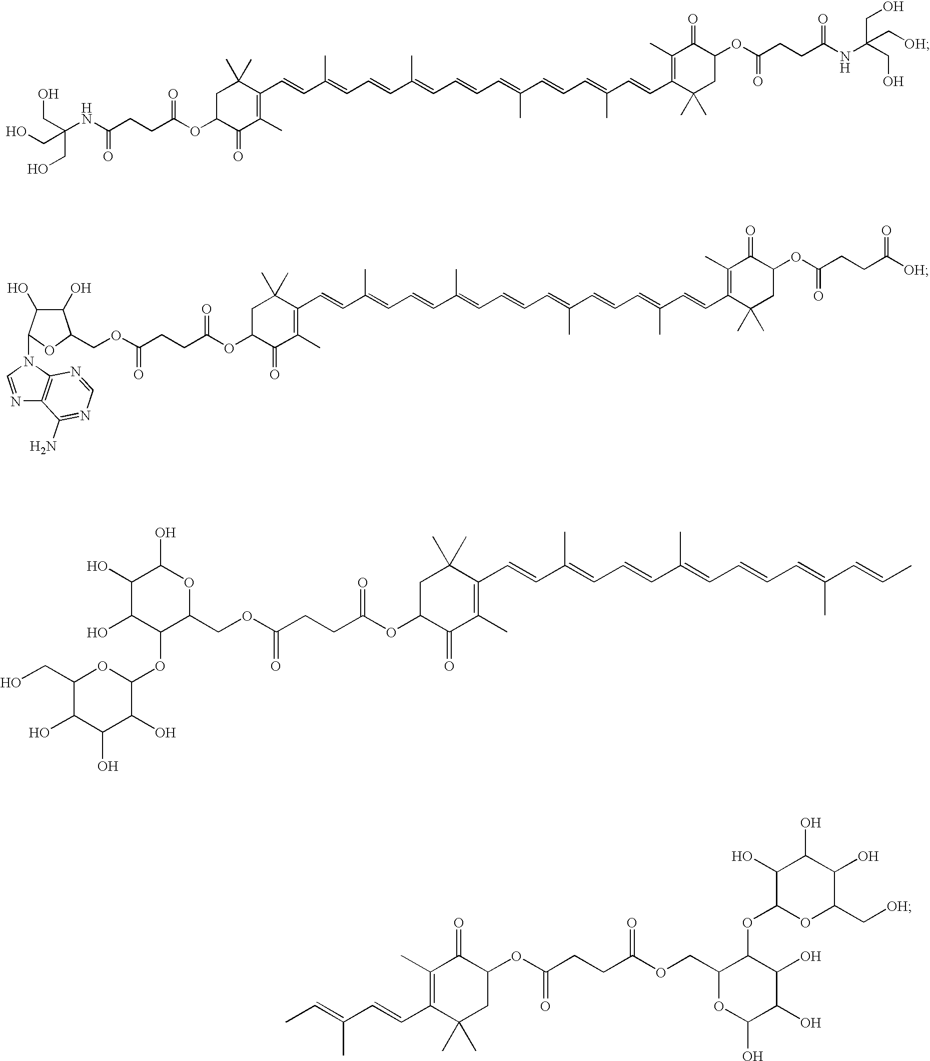

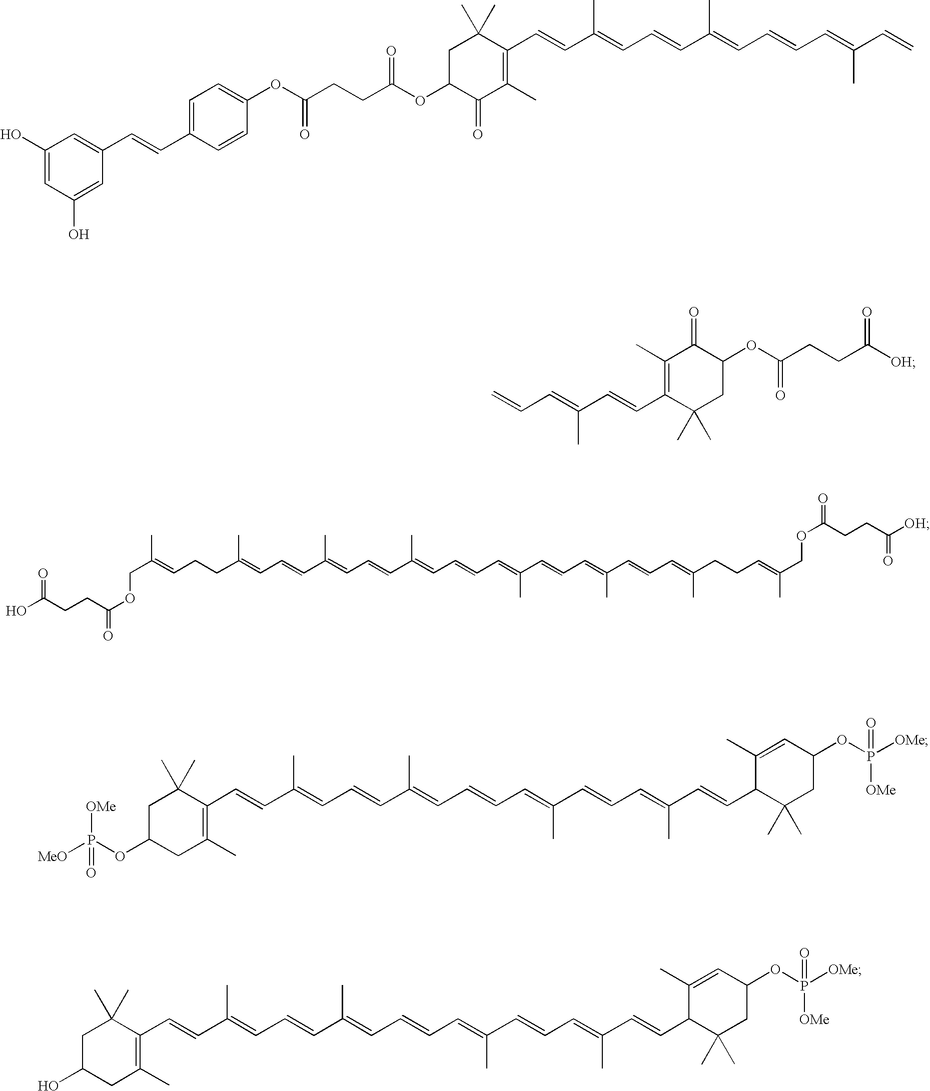

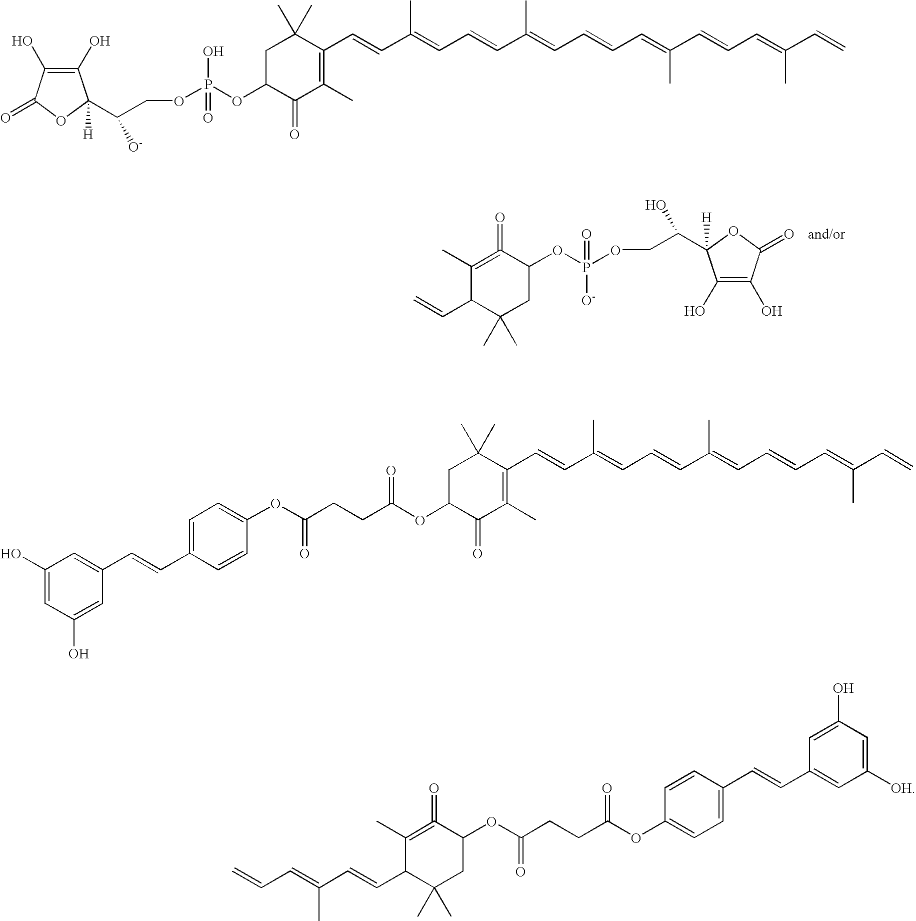

In some embodiments, a chemical compound may include a disuccinic acid ester carotenoid derivative having the structure (160)

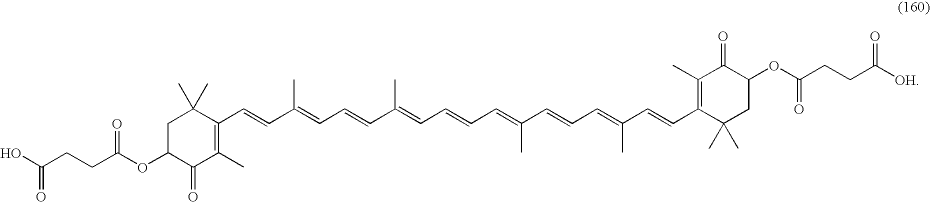

In some embodiments, a chemical compound may include a disodium salt disuccinic acid ester carotenoid derivative having the structure (162)

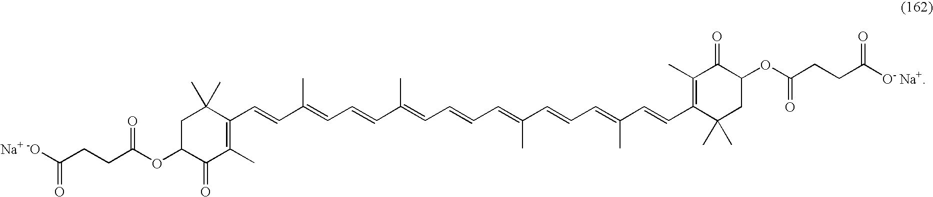

Compounds described herein embrace isomers mixtures, racemic, optically active, and optically inactive stereoisomers and compounds. Carotenoid analogs or derivatives may have increased water solubility and/or water dispersibility relative to some or all known naturally occurring carotenoids. In some embodiments, one or more co-antioxidants may be coupled to a carotenoid or carotenoid derivative or analog.

In some embodiments, carotenoid analogs or derivatives may be employed in “self-formulating” aqueous solutions, in which the compounds spontaneously self-assemble into macromolecular complexes. These complexes may provide stable formulations in terms of shelf life. The same formulations may be parenterally administered, upon which the spontaneous self-assembly is overcome by interactions with serum and/or tissue components in vivo.

Some specific embodiments may include phosphate, succinate, co-antioxidant (e.g., Vitamin C, Vitamin C analogs, Vitamin C derivatives, Vitamin E, Vitamin E analogs, Vitamin E derivatives, or flavonoids), or combinations thereof derivatives or analogs of carotenoids. Flavonoids may include, for example, quercetin, xanthohumol, isoxanthohumol, or genistein. Derivatives or analogs may be derived from any known carotenoid (naturally or synthetically derived). Specific examples of naturally occurring carotenoids which compounds described herein may be derived from include for example zeaxanthin, lutein, lycophyll, astaxanthin, and lycopene.

The synthesis of water-soluble and/or water-dispersible carotenoids (e.g., C40) analogs or derivatives—as potential parenteral agents for clinical applications may improve the injectability of these compounds as therapeutic agents, a result perhaps not achievable through other formulation methods. The methodology may be extended to carotenoids with fewer than 40 carbon atoms in the molecular skeleton and differing ionic character. The methodology may be extended to carotenoids with greater than 40 carbon atoms in the molecular skeleton. The methodology may be extended to non-symmetric carotenoids. The aqueous dispersibility of these compounds allows proof-of-concept studies in model systems (e.g. cell culture), where the high lipophilicity of these compounds previously limited their bioavailability and hence proper evaluation of efficacy. Esterification or etherification may be useful to increase oral bioavailability, a fortuitous side effect of the esterification process, which can increase solubility in gastric mixed micelles. The net overall effect is an improvement in potential clinical utility for the lipophilic carotenoid compounds as therapeutic agents.

In some embodiments, the principles of retrometabolic drug design may be utilized to produce novel soft drugs from the asymmetric parent carotenoid scaffold (e.g., RRR-lutein (β,ε-carotene-3,3′-diol)). For example, lutein scaffold for derivatization was obtained commercially as purified natural plant source material, and was primarily the RRR-stereoisomer (one of 8 potential stereoisomers). Lutein (Scheme 1) possesses key characteristics-similar to starting material astaxanthin-which make it an ideal starting platform for retrometabolic syntheses: (1) synthetic handles (hydroxyl groups) for conjugation, and (2) an excellent safety profile for the parent compound.

In some embodiments, carotenoid analogs or derivatives may have increased water solubility and/or water dispersibility relative to some or all known naturally occurring carotenoids.

In some embodiments, the carotenoid derivatives may include compounds having a structure including a polyene chain (i.e., backbone of the molecule). The polyene chain may include between about 5 and about 15 unsaturated bonds. In certain embodiments, the polyene chain may include between about 7 and about 12 unsaturated bonds. In some embodiments a carotenoid derivative may include 7 or more conjugated double bonds to achieve acceptable antioxidant properties.

In some embodiments, decreased antioxidant properties associated with shorter polyene chains may be overcome by increasing the dosage administered to a subject or patient.

In some embodiments, the carotenoid derivatives or analogs may be synthesized from naturally occurring carotenoids. In some embodiments, the carotenoid derivatives may be synthesized from any naturally occurring carotenoid including one or more alcohol substituents. In other embodiments, the carotenoid derivatives may be synthesized from a derivative of a naturally occurring carotenoid including one or more alcohol substituents. The synthesis may result in a single stereoisomer. The synthesis may result in a single geometric isomer of the carotenoid derivative. The synthesis/synthetic sequence may include any prior purification or isolation steps carried out on the parent carotenoid.

In some embodiments, a synthesis may be a total synthesis using methods described herein to synthesize carotenoid derivatives and/or analogs. An example may include, but is not limited to, a 3S,3′S all-E carotenoid derivative, where the parent carotenoid is astaxanthin. The synthetic sequence may include protecting and subsequently deprotecting various functionalities of the carotenoid and/or substituent precursor. When derivates or analogs are prepared from alcohol-functionalized carotenoids, a base catalyzed reaction may be used to react the alcohol functional groups with the substituent precursor. Substituent precursors include precursors that include a functional group that may act as a leaving group for a substitution reaction. The base may include any non-nucleophilic base known to one skilled in the art such as, for example, tertiary amines, pyridine, pyrrolidine, etc. The alcohol may act as a nucleophile reacting with the substituent precursor, displacing the leaving group. Leaving groups may include, but are not limited to, I, Cl, Br, tosyl, brosyl, mesyl, or trifyl. These are only a few examples of leaving groups that may be used, many more are known and would be apparent to one skilled in the art. In some embodiments, a base may be used to deprotonate the alcohol. For example, reaction with alkyl lithium bases, alkali metal hydroxide, or alkali metal alcohol salts may deprotonate a hydroxy group of the carotenoid. In other examples the leaving group may be internal and may subsequently be included in the final structure of the carotenoid derivative, a non-limiting example may include anhydrides or strained cyclic ethers. For example, the alcohol may be reacted with succinic anhydride.

In an embodiment, the disuccinic acid ester of astaxanthin may be further converted to the disodium salt. Examples of synthetic sequences for the preparation of some of the specific embodiments depicted are described in the Examples section. The example depicted below is a generic non-limiting example of a synthetic sequence for the preparation of astaxanthin carotenoid derivatives.

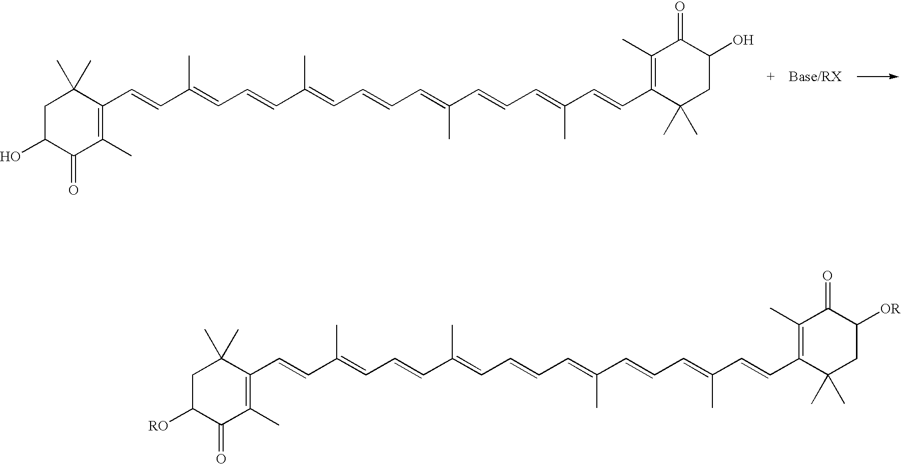

In some embodiments, one or more of the conversions and/or reactions discussed herein may be carried out within one reaction vessel increasing the overall efficiency of the synthesis of the final product. In some embodiments, a product of one reaction during a total synthesis may not be fully worked up before continuing on with the following reaction. In general, fully working up a reaction implies completely isolating and purify the product from a reaction. A reaction may instead only partially be worked up. For example, solid impurities, which fall out of solution during the course of a reaction, may be filtered off and the filtrate washed with solvent to ensure all of the resulting product is washed through and collected. In such a case the resulting collected product still in solution may not be isolated, but may then be combined with another reagent and further transformed. In some cases multiple transformations may be carried out in a single reaction flask simply by adding reagents one at a time without working up intermediate products. These types of “shortcuts” will improve the overall efficiency of a synthesis, especially when dealing with larger quantity reactions (e.g., along the lines of pilot plant scale and/or plant scale).

In some embodiments, an alcohol-functionalized carotenoid may provide a skeleton with a useful handle with which to appropriately derivative a carotenoid based water dispersible end product. The example depicted above is a generic non-limiting example; examples depicted in Schemes 1 and 2 provide more specific examples of the synthesis of water-soluble and/or water-dispersible carotenoid analogs or derivatives. Schemes 1 and 2 depict the syntheses of two water-dispersible lutein derivatives, the sodium salts of lutein disuccinate and lutein diphosphate. Derivatizing hydrophobic carotenoids may impart water-dispersibility.

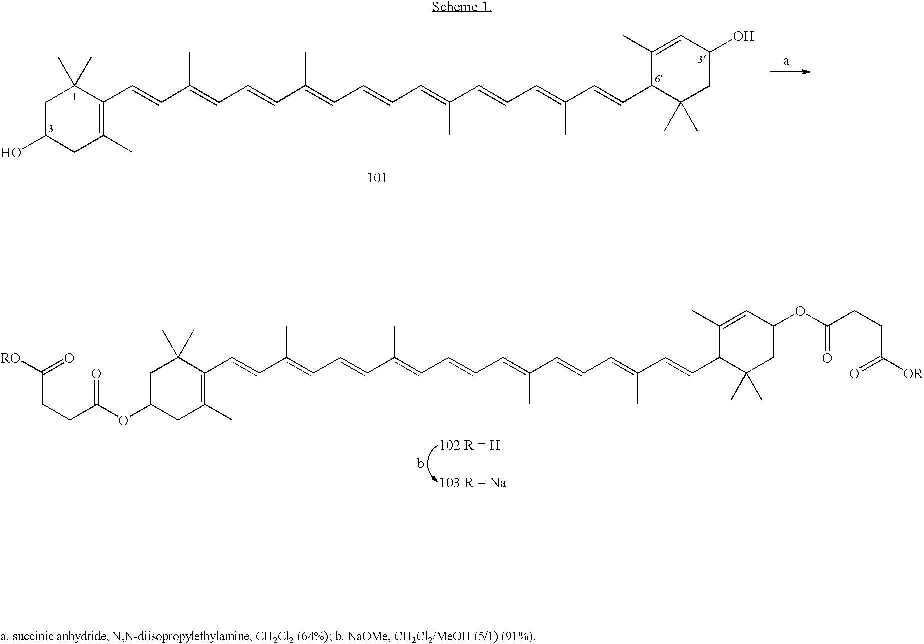

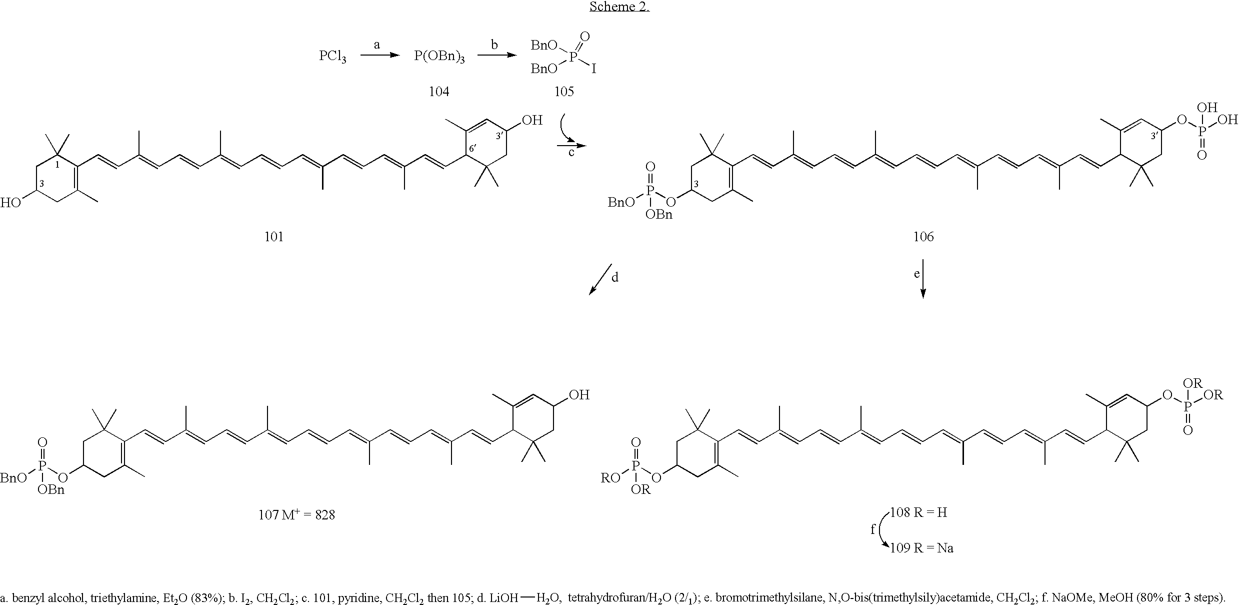

As seen in Scheme 1, the synthesis of disuccinate salt 103 began with succinylation of natural source lutein using succinic anhydride and Hünig base (N,N′-diisopropylethylamine). Reactions may be run in polar organic solvents. Disuccinylation of lutein was optimized by running the reaction in a concentrated fashion and using modest excesses of anhydride and base. Using high concentrations of reagents may allow easier extraction of impurities and side products once the reaction is complete. Aqueous acidic workup yielded disuccinate 102, such that excess reagents and reaction byproducts were removed by copiously extracting the organic layer with dilute HCl. The resulting viscous, red-orange oil was washed or slurried with hexanes to remove non-polar impurities. A successfully functionalized carotenoid may be transformed into an ionic salt derivative or analog in order to increase the water solubility. A carotenoid may be transformed into an ionic salt derivative or analog by reacting the carotenoid with a base. Bases may include alkali metal hydroxides (e.g., sodium hydroxide) or tertiary amines (e.g., triethylamine). In some embodiments, bases, upon deprotonation of one or more moieties of the carotenoid may result in by products which are easily removed (e.g., removed under reduced pressure, extracted). The water-dispersible derivative 103 was generated by treating compound 102 with methanolic sodium methoxide. The reaction was quenched with water and the resulting red-orange aqueous layer was first extracted with Et₂O, then lyophilized to provide the sodium salt in good yield.

In some embodiments, a carotenoid may be phosphorylated to increase water solubility and/or dispersibility. In some embodiments, a carotenoid may be diphosphorylated to increase water solubility and/or dispersibility. Successful diphosphorylation of lutein may be achieved using dimethyl phosphoroiodidate. Dimethyl phosphoroiodidate may be formed in situ. Dimethyl phosphoroiodidate may be formed by reacting commercially available trimethyl phosphite with iodine. In some embodiments, a certain degree of success in removing all four diphosphate methyl groups may be realized when using bromotrimethylsilane in the presence of N,O-bis(trimethylsilyl)acetamde. However, this deprotection protocol may not be optimal in that methyl group dealkylation was usually accompanied by the significant decomposition of lutein phosphate.

In some embodiments, a three-step method to provide the tetra-sodium salt of lutein diphosphate 109 may be achieved using benzyl esters as protecting groups for the lutein phosphoric acids (Scheme 2). Lutein (e.g., natural source) may be phosphorylated using dibenzyl phosphoroiodidate. Dibenzyl phosphoroiodidate may be formed in situ. Dibenzyl phosphoroiodidate may be formed by reacting tribenzyl phosphite with iodine. As seen in Scheme 2, tribenzyl phosphite may be prepared by the addition of benzyl alcohol to phosphorus trichloride in the presence of triethylamine. In some embodiments, silica gel chromatography of the crude reaction mixture may yield tribenzyl phosphite in good yield. Compound 106 was formed by treating lutein with freshly prepared dibenzyl phosphoroiodidate in the presence of pyridine. Aqueous workup of the reaction followed by the removal of pyridine by azeotropic distillation using toluene may provide a crude red oil. Contaminations, excess reagents, and reaction byproducts may be removed during work up of the reaction or at a later time (e.g., after a subsequent reaction). Non-polar impurities may be removed from the crude product mixture by alternately washing or slurrying with hexanes and Et₂O to give 106.

In some embodiments, dealkylation of one or more of the four benzyl esters of the phosphoric acid moieties may occur during the phosphorylation reaction. Dealkylation may occurr at the more sensitive allylic 3′ phosphate positions. As seen in Scheme 2, the attempted removal of the phosphoric acid benzyl esters of 106 using LiOH—H₂O may result in the generation of a less polar product versus compound 106, exhibiting a molecular ion of 828 as noted by LC/MS analysis. Under these reaction conditions, dephosphorylation at one of the two hydroxyls of the lutein derivative may occur rather than the desired debenzylation to give compound 107. Such data indirectly support compound 106's structure and thus the occurrence of bis-dealkylation at one phosphate versus mono-dealkylation at both phosphates as an additional result of the phosphorylation of lutein. If mono-dealkylation at both phosphates occurred during phosphorylation, then treatment of the resulting product with LiOH—H₂O would have produced a lutein derivative possessing one phosphoric acid containing only one benzyl ester, exhibiting a molecular ion of 738 upon LC/MS analysis.

In some embodiments, successful dealkylation of the phosphate protecting groups of 106 may be achieved using bromotrimethylsilane in the presence of N,O-bis(trimethylsilyl)acetamide (see Scheme 2). A significant amount of excess reagents and reaction byproducts may be removed from the resulting red oil by alternately washing or slurrying the crude mixture with ethyl acetate and CH₂Cl₂ to provide diphosphate 108 as an orange oil.

In some embodiments, the sodium salt of lutein diphosphate (109) may be generated by treating 108 with methanolic sodium methoxide (see Scheme 2). The resulting crude orange solid may be washed or slurried with methanol and then dissolved in water. The aqueous layer may be extracted first with CH₂Cl₂, then with ethyl acetate, and again with CH₂Cl₂. Lyophilization of the red-orange aqueous solution may give the sodium salt as an orange, hygroscopic solid. The phosphorylation process may provide the desired water-dispersible lutein derivative 109 in good yield over the three steps.

The synthetic preparation of carotenoid derivatives or analogs such as disodium disuccinate astaxanthin 162 at multigram scale (e.g., 200 g to 1 kg) is necessary if one wishes to produce these molecules commercially. Synthetic modifications of carotenoids, with the goal of increasing aqueous solubility and/or dispersibility, have been sparingly reported in the literature. At the time process development began, surveys of the peer-reviewed and patent literature indicated that neither a synthetic sequence nor an efficient process for the synthesis of 160 or 162 had been reported. Therefore, the bench-scale synthetic sequence and later the scale-up to multigram scale were optimized to improve both the yield and purity of the desired compound. Examples of synthetic preparation of carotenoids and carotenoid derivatives or analogs are illustrated in U.S. Patent Application Ser. No. 60/615,032 filed on Oct. 1, 2004, entitled “METHODS FOR SYNTHESIS OF CAROTENOIDS, INCLUDING ANALOGS, DERIVATIVES, AND SYNTHETIC AND BIOLOGICAL INTERMEDIATES” to Lockwood et al. which is incorporated by reference as if fully set forth herein.

The disodium disuccinate derivatives of synthetic astaxanthin were successfully synthesized in gram amounts and at high purity (>90%) area under the curve (AUC) by HPLC. The compound in “racemic” form demonstrated water “dispersibility” of 8.64 mg/mL, a significant improvement over the parent compound astaxanthin, which is insoluble in water. Initial biophysical characterization demonstrated that Cardax™ derivatives (as both the statistical mixture of stereoisomers and as individual stereoisomers) were potent direct scavengers of superoxide anion in the aqueous phase, the first such description in this model system for a C40 carotenoid. Plasma-protein binding studies in vitro revealed that the meso-(3R,3′S)-disodium disuccinate astaxanthin derivative bound immediately and preferentially to human serum albumin (HSA) at a binding site, suggesting that beneficial ligand-binding associations might take place in vivo after parenteral administration of the compound. The single- and multiple-dose pharmacokinetics of an oral preparation of the racemic compound (in lipophilic emulsion) were then investigated in a murine model, and significant plasma and tissue levels of nonesterified astaxanthin were achieved. Proof-of-concept studies in ischemia-reperfusion injury performed in rodents subsequently revealed that intravenous pretreatment with Cardax™ was significantly cardioprotective and achieved myocardial salvage in this experimental infarction model (e.g., up to 56% at the highest dose tested). The test material for three of the studies described above was obtained from a single pilot batch of compound (>200 g single batch at >97% purity by HPLC).

In some embodiments, it may be advantageous to be able to efficiently separate out individual stereoisomers of a racemic mixture of a chemical compound. Efficiently separating out individual stereoisomers on a relatively large scale may advantageously increase availability of starting materials.

In some embodiments, chromatographic separation techniques may be used to separate stereoisomers of a racemic mixture. In some embodiments pure optically active stereoisomers may be reacted with a mixture of stereoisomers of a chemical compound to form a mixture of diastereomers. Diastereomers may have different physical properties as opposed to stereoisomers, thus making it easier to separate diastereomers.

For example it may be advantageous to separate out stereoisomers from a racemic mixture of astaxanthin. In some embodiments, astaxanthin may be coupled to an optically active compound (e.g., dicamphanic acid). Coupling astaxanthin to optically active compounds produces diastereomers with different physical properties. The diastereomers produced may be separated using chromatographic separation techniques as described herein.

Bulk chromatographic separation of the diastereomeric dicamphanic acid ester(s) of synthetic astaxanthin at preparative chromatography scale was performed to subsequently make gram-scale quantities of each stereoisomer of disodium disuccinate ester astaxanthin.

As used herein the terms “structural carotenoid analogs or derivatives” may be generally defined as carotenoids and the biologically active structural analogs or derivatives thereof. “Derivative” in the context of this application is generally defined as a chemical substance derived from another substance either directly or by modification or partial substitution. “Analog” in the context of this application is generally defined as a compound that resembles another in structure but is not necessarily an isomer. Typical analogs or derivatives include molecules which demonstrate equivalent or improved biologically useful and relevant function, but which differ structurally from the parent compounds. Parent carotenoids are selected from the more than 700 naturally occurring carotenoids described in the literature, and their stereo- and geometric isomers. Such analogs or derivatives may include, but are not limited to, esters, ethers, carbonates, amides, carbamates, phosphate esters and ethers, sulfates, glycoside ethers, with or without spacers (linkers).

As used herein the terms “the synergistic combination of more than one structural analog or derivative or synthetic intermediate of carotenoids” may be generally defined as any composition including one structural carotenoid analog or derivative or synthetic intermediate combined with one or more other structural carotenoid analogs or derivatives or synthetic intermediate or co-antioxidants, either as derivatives or in solutions and/or formulations.

In some embodiments, techniques described herein may be applied to the inhibition and/or amelioration of any disease or disease state related to reactive oxygen species (“ROS”) and other radical and non-radical species.

In some embodiments, techniques described herein may be applied to the inhibition and/or amelioration of inflammation, including but not limited to ischemic reperfusion injury of a tissue.

An embodiment may include the administration of structural carotenoid analogs or derivatives or synthetic intermediates alone or in combination to a subject such that the occurrence of inflammation is thereby inhibited and/or ameliorated. The structural carotenoid analogs or derivatives or synthetic intermediates may be water-soluble and/or water dispersible derivatives. The carotenoid derivatives may include any substituent that substantially increases the water solubility of the naturally occurring carotenoid. The carotenoid derivatives may retain and/or improve the antioxidant properties of the parent carotenoid. The carotenoid derivatives may retain the non-toxic properties of the parent carotenoid. The carotenoid derivatives may have increased bioavailability, relative to the parent carotenoid, upon administration to a subject. The parent carotenoid may be naturally occurring.

Another embodiments may include the administration of a composition comprised of the synergistic combination of more than one structural analog or derivative or synthetic intermediate of carotenoids to a subject such that the occurrence of a proliferative disorder is thereby reduced. The composition may be a “racemic” (i.e. mixture of the potential stereoisomeric forms) mixture of carotenoid derivatives. Included as well are pharmaceutical compositions comprised of structural analogs or derivatives or synthetic intermediates of carotenoids in combination with a pharmaceutically acceptable carrier. In one embodiment, a pharmaceutically acceptable carrier may be serum albumin. In one embodiment, structural analogs or derivatives or synthetic intermediates of carotenoids may be complexed with human serum albumin (i.e., HSA) in a solvent. HSA may act as a pharmaceutically acceptable carrier.

In some embodiments, a single stereoisomer of a structural analog or derivative or synthetic intermediate of carotenoids may be administered to a human subject in order to ameliorate a pathological condition. Administering a single stereoisomer of a particular compound (e.g., as part of a pharmaceutical composition) to a human subject may be advantageous (e.g., increasing the potency of the pharmaceutical composition). Administering a single stereoisomer may be advantageous due to the fact that only one isomer of potentially many may be biologically active enough to have the desired effect.

In some embodiments, compounds described herein may be administered in the form of nutraceuticals. “Nutraceuticals” as used herein, generally refers to dietary supplements, foods, or medical foods that: 1. possess health benefits generally defined as reducing the risk of a disease or health condition, including the management of a disease or health condition or the improvement of health; and 2. are safe for human consumption in such quantity, and with such frequency, as required to realize such properties. Generally a nutraceutical is any substance that is a food or a part of a food and provides medical or health benefits, including the prevention and treatment of disease. Such products may range from isolated nutrients, dietary supplements and specific diets to genetically engineered designer foods, herbal products, and processed foods such as cereals, soups and beverages. It is important to note that this definition applies to all categories of food and parts of food, ranging from dietary supplements such as folic acid, used for the prevention of spina bifida, to chicken soup, taken to lessen the discomfort of the common cold. This definition also includes a bio-engineered designer vegetable food, rich in antioxidant ingredients, and a stimulant functional food or pharmafood. Within the context of the description herein where the composition, use and/or delivery of pharmaceuticals are described nutraceuticals may also be composed, used, and/or delivered in a similar manner where appropriate.

In some embodiments, compositions may include all compositions of 1.0 gram or less of a particular structural carotenoid analog, in combination with 1.0 gram or less of one or more other structural carotenoid analogs or derivatives or synthetic intermediates and/or co-antioxidants, in an amount which is effective to achieve its intended purpose. While individual subject needs vary, determination of optimal ranges of effective amounts of each component is with the skill of the art. Typically, a structural carotenoid analog or derivative or synthetic intermediates may be administered to mammals, in particular humans, orally at a dose of 5 to 100 mg per day referenced to the body weight of the mammal or human being treated for a particular disease. Typically, a structural carotenoid analog or derivative or synthetic intermediate may be administered to mammals, in particular humans, parenterally at a dose of between 5 to 1000 mg per day referenced to the body weight of the mammal or human being treated for a particular disease. In other embodiments, about 100 mg of a structural carotenoid analog or derivative or synthetic intermediate is either orally or parenterally administered to treat or prevent disease.

The unit oral dose may comprise from about 0.25 mg to about 1.0 gram, or about 5 to 25 mg, of a structural carotenoid analog. The unit parenteral dose may include from about 25 mg to 1.0 gram, or between 25 mg and 500 mg, of a structural carotenoid analog. The unit intracoronary dose may include from about 25 mg to 1.0 gram, or between 25 mg and 100 mg, of a structural carotenoid analog. The unit doses may be administered one or more times daily, on alternate days, in loading dose or bolus form, or titrated in a parenteral solution to commonly accepted or novel biochemical surrogate marker(s) or clinical endpoints as is with the skill of the art.

In addition to administering a structural carotenoid analog or derivative or synthetic intermediate as a raw chemical, the compounds may be administered as part of a pharmaceutical preparation containing suitable pharmaceutically acceptable carriers, preservatives, excipients and auxiliaries which facilitate processing of the structural carotenoid analog or derivative or synthetic intermediates which may be used pharmaceutically. The preparations, particularly those preparations which may be administered orally and which may be used for the preferred type of administration, such as tablets, softgels, lozenges, dragees, and capsules, and also preparations which may be administered rectally, such as suppositories, as well as suitable solutions for administration by injection or orally or by inhalation of aerosolized preparations, may be prepared in dose ranges that provide similar bioavailability as described above, together with the excipient. While individual needs may vary, determination of the optimal ranges of effective amounts of each component is within the skill of the art.

The pharmaceutical preparations may be manufactured in a manner which is itself known to one skilled in the art, for example, by means of conventional mixing, granulating, dragee-making, softgel encapsulation, dissolving, extracting, or lyophilizing processes. Thus, pharmaceutical preparations for oral use may be obtained by combining the active compounds with solid and semi-solid excipients and suitable preservatives, and/or co-antioxidants. Optionally, the resulting mixture may be ground and processed. The resulting mixture of granules may be used, after adding suitable auxiliaries, if desired or necessary, to obtain tablets, softgels, lozenges, capsules, or dragee cores.

Suitable excipients may be fillers such as saccharides (e.g., lactose, sucrose, or mannose), sugar alcohols (e.g., mannitol or sorbitol), cellulose preparations and/or calcium phosphates (e.g., tricalcium phosphate or calcium hydrogen phosphate). In addition binders may be used such as starch paste (e.g., maize or corn starch, wheat starch, rice starch, potato starch, gelatin, tragacanth, methyl cellulose, hydroxypropylmethylcellulose, sodium carboxymethylcellulose, and/or polyvinyl pyrrolidone). Disintegrating agents may be added (e.g., the above-mentioned starches) as well as carboxymethyl-starch, cross-linked polyvinyl pyrrolidone, agar, or alginic acid or a salt thereof (e.g., sodium alginate). Auxiliaries are, above all, flow-regulating agents and lubricants (e.g., silica, talc, stearic acid or salts thereof, such as magnesium stearate or calcium stearate, and/or polyethylene glycol, or PEG). Dragee cores are provided with suitable coatings, which, if desired, are resistant to gastric juices. Softgelatin capsules (“softgels”) are provided with suitable coatings, which, typically, contain gelatin and/or suitable edible dye(s). Animal component-free and kosher gelatin capsules may be particularly suitable for the embodiments described herein for wide availability of usage and consumption. For this purpose, concentrated saccharide solutions may be used, which may optionally contain gum arabic, talc, polyvinyl pyrrolidone, polyethylene glycol (PEG) and/or titanium dioxide, lacquer solutions and suitable organic solvents or solvent mixtures, including dimethylsulfoxide (DMSO), tetrahydrofuran (THF), acetone, ethanol, or other suitable solvents and co-solvents. In order to produce coatings resistant to gastric juices, solutions of suitable cellulose preparations such as acetylcellulose phthalate or hydroxypropylmethyl-cellulose phthalate, may be used. Dye stuffs or pigments may be added to the tablets or dragee coatings or softgelatin capsules, for example, for identification or in order to characterize combinations of active compound doses, or to disguise the capsule contents for usage in clinical or other studies.

Other pharmaceutical preparations that may be used orally include push-fit capsules made of gelatin, as well as soft, thermally sealed capsules made of gelatin and a plasticizer such as glycerol or sorbitol. The push-fit capsules may contain the active compounds in the form of granules that may be mixed with fillers such as, for example, lactose, binders such as starches, and/or lubricants such as talc or magnesium stearate and, optionally, stabilizers and/or preservatives. In soft capsules, the active compounds may be dissolved or suspended in suitable liquids, such as fatty oils such as rice bran oil or peanut oil or palm oil, or liquid paraffin. In some embodiments, stabilizers and preservatives may be added.

In some embodiments, pulmonary administration of a pharmaceutical preparation may be desirable. Pulmonary administration may include, for example, inhalation of aerosolized or nebulized liquid or solid particles of the pharmaceutically active component dispersed in and surrounded by a gas.

Possible pharmaceutical preparations, which may be used rectally, include, for example, suppositories, which consist of a combination of the active compounds with a suppository base. Suitable suppository bases are, for example, natural or synthetic triglycerides, or paraffin hydrocarbons. In addition, it is also possible to use gelatin rectal capsules that consist of a combination of the active compounds with a base. Possible base materials include, for example, liquid triglycerides, polyethylene glycols, or paraffin hydrocarbons.

Suitable formulations for parenteral administration include, but are not limited to, aqueous solutions of the active compounds in water-soluble and/or water dispersible form, for example, water-soluble salts, esters, carbonates, phosphate esters or ethers, sulfates, glycoside ethers, together with spacers and/or linkers. Suspensions of the active compounds as appropriate oily injection suspensions may be administered, particularly suitable for intramuscular injection. Suitable lipophilic solvents, co-solvents (such as DMSO or ethanol), and/or vehicles including fatty oils, for example, rice bran oil or peanut oil and/or palm oil, or synthetic fatty acid esters, for example, ethyl oleate or triglycerides, may be used. Aqueous injection suspensions may contain substances that increase the viscosity of the suspension including, for example, sodium carboxymethyl cellulose, sorbitol, dextran, and/or cyclodextrins. Cyclodextrins (e.g., β-cyclodextrin) may be used specifically to increase the water solubility for parenteral injection of the structural carotenoid analog. Liposomal formulations, in which mixtures of the structural carotenoid analog or derivative with, for example, egg yolk phosphotidylcholine (E-PC), may be made for injection. Optionally, the suspension may contain stabilizers, for example, antioxidants such as BHT, and/or preservatives, such as benzyl alcohol.

### EXAMPLES

Having now described the invention, the same will be more readily understood through reference to the following example(s), which are provided by way of illustration, and are not intended to be limiting of the present invention.

General. Natural source lutein (90%) was obtained from ChemPacific, Inc. (Baltimore, Md.) as a red-orange solid and was used without further purification. All other reagents and solvents used were purchased from Acros (New Jersey, USA) and were used without further purification. All reactions were performed under N₂ atmosphere. All flash chromatographic purifications were performed on Natland International Corporation 230-400 mesh silica gel using the indicated solvents. LC/MS (APCI) and LC/MS (ESI) were recorded on an Agilent 1100 LC/MSD VL system; column: Zorbax Eclipse XDB-C18 Rapid Resolution (4.6×75 mm, 3.5 μm, USUT002736); temperature: 25° C.; starting pressure: 105 bar; flow rate: 1.0 mL/min; mobile phase (% A=0.025% trifluoroacetic acid in H₂O, % B=0.025% trifluoroacetic acid in acetonitrile) Gradient program: 70% A/30% B (start), step gradient to 50% B over 5 min, step gradient to 98% B over 8.30 min, hold at 98% B over 25.20 min, step gradient to 30% B over 25.40 min; PDA Detector: 470 nm. The presence of trifluoroacetic acid in the LC eluents acts to protonate synthesized lutein disuccinate and diphosphate salts to give the free di-acid forms, yielding M⁺=768 for the disuccinate salt sample and M⁺=728 for the diphosphate salt sample in MS analyses. LRMS:+mode; ESI: electrospray chemical ionization, ion collection using quadrapole; APCI: atmospheric pressure chemical ionization, ion collection using quadrapole. MS (ESI-IT) was recorded on a HCT plus Bruker Daltonics Mass Spectrometer system, LRMS:+mode; ESI-IT: electrospray chemical ionization, ion collection using ion trap. ¹H NMR analyses were attempted on Varian spectrometers (300 and 500 MHz). NMR analyses of natural source lutein as well as synthesized lutein derivatives yielded only partially discernable spectra, perhaps due to the presence of interfering impurities (natural source lutein), or due to aggregation (natural source lutein and derivatives). In attempts to circumvent the problems associated with NMR analyses, samples were prepared using mixtures of deuterated solvents including methanol/chloroform, methanol/water, methyl sulfoxide/water, and chloroform/methanol/water. However, such attempts failed to give useful data.

Natural source lutein (β,ε-carotene-3,3′-diol), 1. LC/MS (ESI): 9.95 min (2.78%), λₘₐₓ226 nm (17%), 425 nm (100%); 10.58 min (3.03%), λₘₐₓ 225 nm (21%), 400 nm (100%); 11.10 min (4.17%), λₘₐₓ 225 nm (16%); 447 nm (100%); 12.41 min (90.02%), λₘₐₓ 269 nm (14%), 447 nm (100%), m/z 568 M⁺(69%), 551 [M−H₂O+H]⁺ (100%), 533 [M−2H₂O+H]⁺ (8%)

β,ε-carotenyl 3,3′-disuccinate, 2. To a solution of natural source lutein (1) (0.50 g, 0.879 mmol) in CH₂Cl₂ (8 mL) was added N,N-diisopropylethylamine (3.1 mL, 17.58 mmol) and succinic anhydride (0.88 g, 8.79 mmol). The solution was stirred at RT overnight and then diluted with CH₂Cl₂ and quenched with water/1 M HCl (5/1). The aqueous layer was extracted two times with CH₂Cl₂ and the combined organic layer was washed three times with cold water/1 M HCl (5/1), dried over Na₂SO₄, and concentrated. The resulting red-orange oil was washed (slurried) three times with hexanes to yield disuccinate 2 (0.433 g, 64%) as a red-orange solid; LC/MS (APCI): 10.37 min (4.42%), λₘₐₓ 227 nm (56%), 448 nm (100%), m/z 769 [M+H]⁺ (8%), 668 [M−C₄O₃H₄]⁺ (9%), 637 (36%), 138 (100%); 11.50 min (92.40%), λₘₐₓ 269 nm (18%), 447 nm (100%), m/z 769 [M+H]⁺ (7%), 668 [M−C₄O₃H₄]⁺ (9%), 651 (100%); 12.03 min (3.18%) λₘₐₓ 227nm (55%), 446 nm (100%), m/z 668 [M−C₄O₃H₄]⁺ (15%), 550 (10%), 138 (100%)

β,ε-carotenyl 3,3′-disuccinate sodium salt, 3. To a solution of disuccinate 2 (0.32 g, 0.416 mmol) in CH₂Cl₂/methanol (5 mL/1 mL ) at 0° C. was added drop-wise sodium methoxide (25% wt in methanol; 0.170 mL, 0.748 mmol). The solution was stirred at RT overnight and then quenched with water and stirred for 5 min. The solution was then concentrated and the aqueous layer was washed four times with Et₂O. Lyophilization of the clear, red-orange aqueous solution yielded 3 (0.278 g, 91%) as an orange, hygroscopic solid; LC/MS (APCI): 11.71 min (94.29%), λₘₐₓ 269 nm (18%), 446 nm (100%), m/z 769 [M−2Na+3H]⁺ (8%), 668 [M−2Na+2H−C₄O₃H₄]⁺ (6%), 651 (100%); 12.74 min (5.71%), λₘₐₓ 227 nm (30%), 269 nm (18%), 332 nm (39%), 444 nm (100%), m/z 768 [M−2Na+2H]⁺ (2%), 668 [M−2Na+2H−C₄O₃H₄]⁺ (3%), 651 (12%), 138 (100%)

Tribenzyl phosphite, 4. To a well-stirred solution of phosphorus trichloride (1.7 mL, 19.4 mmol) in Et₂O (430 mL) at 0° C. was added dropwise a solution of triethylamine (8.4 mL, 60.3 mmol) in Et₂O (20 mL), followed by a solution of benzyl alcohol (8.1 mL, 77.8 mmol) in Et₂O (20 mL). The mixture was stirred at 0° C. for 30 min and then at RT overnight. The mixture was filtered and the filtrate concentrated to give a colorless oil. Silica chromatography (hexanes/Et₂O/triethylamine, 4/1/1%) of the crude product yielded 4 (5.68 g, 83%) as a clear, colorless oil that was stored under N₂ at −20° C.; ¹H NMR: δ 7.38 (15H, m), 4.90 (6H, d)

Dibenzyl phosphoroiodidate, 5. To a solution of tribenzyl phosphite (5.43 g, 15.4 mmol) in CH₂Cl₂ (8 mL) at 0° C. was added 12 (3.76 g, 14.8 mmol). The mixture was stirred at 0° C. for 10 min or until the solution became clear and colorless. The solution was then stirred at RT for 10 min and used directly in the next step.

3-(Bis benzyl-phosphoryloxy)-3′-(phosphoryloxy)-β,ε-carotene, 6. To a solution of natural source lutein (1) (0.842 g, 1.48 mmol) in CH₂Cl₂ (8 mL) was added pyridine (4.8 mL, 59.2 mmol). The solution was stirred at 0° C. for 5 min and then freshly prepared 5 (14.8 mmol) in CH₂Cl₂ (8 mL) was added drop-wise to the mixture at 0° C. The solution was stirred at 0° C. for 1 h and then diluted with CH₂Cl₂ and quenched with brine. The aqueous layer was extracted twice with CH₂Cl₂ and the combined organic layer was washed once with brine, then dried over Na₂SO₄ and concentrated. Pyridine was removed from the crude red oil by azeotropic distillation using toluene. The crude product was alternately washed (slurried) twice with hexanes and Et₂O to yield 6 as a red oil, used in the next step without further purification; LC/MS (ESI): 9.93 min (44.78%), λₘₐₓ 267 nm (33%), 444 nm (100%), m/z 890 [M−H₂O ]⁺ (8%), 811 [M−PO₃H−H₂+H]⁺ (73%), 533 (100%); 9.99 min (29.0%), λₘₐₓ 268 nm (24%). 446 nm (100%), m/z 890 [M−H₂O]⁺ (6%), 811 [M−PO₃H−H₂O +H]⁺ (72%), 533 (100%); 10.06 min (26.23%), λₘₐₓ 266 nm (15%), 332 nm (22%), 444 nm (100%), m/z 890 [M−H₂O ]⁺ (5%), 811 [M−PO₃H−H₂O+H]⁺ (90%), 533 (100%)

3-(Bis benzyl-phosphoryloxy)-3′-hydroxy-β,ε-carotene, 7. To a solution of 6 (0.033 mmol) in tetrahydrofuran/water (1 mL/0.5 mL) at 0° C. was added LiOH-H20 (0.003 g, 0.073 mmol). The solution stirred at RT for 1 h and then quenched with methanol. The crude reaction mixture was analyzed by LC/MS; LC/MS (ESI): 10.02 min (40.60%), λₘₐₓ 266 nm (12%), 333 nm (25%), 445 nm (100%), m/z 890 [M−H₂O]⁺ (33%), 811 [M−PO₃H−H₂O+H]⁺ (50%), 533 (100%); 16.37 min (49.56%) λₘₐₓ 267 nm (16%), 332 nm (27%), 446 nm (100%), m/z 828 M⁺ (55%), 550 (44%)

3,3′-Diphosphoryloxy-β,ε-carotene, 8. To a solution of 6 (1.48 mmol) in CH₂Cl₂ (10 mL) at 0° C. was added drop-wise N,O-bis(trimethylsilyl)acetamide (3.7 mL, 14.8 mmol) and then bromotrimethylsilane (1.56 mL, 11.8 mmol). The solution was stirred at 0° C. for 1 h, quenched with methanol, diluted with CH₂Cl₂, and then concentrated. The resulting red oil was alternately washed (slurried) three times with ethyl acetate and CH₂Cl₂ to yield crude phosphate 8 (2.23 g) as a dark orange oil, used in the next step without further purification; LC/MS (ESI): 8.55 min (45.67%), λₘₐₓ 214 mn (25%), 268 nm (28%), 447 nm (100%), m/z 631 [M−PO₃H−H₂O+H]⁺ (30%), 533 (18%), 279 (13%), 138 (87%); 8.95 min (35.0%), λₘₐₓ 217 nm (14%), 268 nm (23%), 448 nm (100%), m/z 631 [M−PO₃H−H₂O+H]⁺ (26%), 533 (32%), 279 (18%), 138 (100%); 9.41 min (9.70%); λₘₐₓ 225 nm (37%), 269 nm (23%), 335 nm (19%), 447 nm (100%), m/z 631 [M−PO₃H−H₂O+H]⁺ (6%), 553 (18%), 279 (13%), 138 (100%)

3,3′-Diphosphoryloxy-β,ε-carotene sodium salt, 9. To a solution of crude 8 (ca 50%; 2.23 g, 3.06 mmol) in methanol (20 mL) at 0° C. was added drop-wise sodium methoxide (25%; 3.5 mL, 15.3 mmol). The solution was stirred at RT for 2h and the resulting orange solid was washed (slurried) three times with methanol. Water was added to the moist solid and the resulting aqueous layer was extracted with CH₂Cl₂, ethyl acetate, and again with CH₂Cl₂. Lyophilization of the clear, red-orange aqueous solution yielded 9 (0.956 g, 80% over 3 steps) as an orange, hygroscopic solid; LC/MS (ESI): 7.81 min (22.34%), λₘₐₓ 215 nm (34%), 268 nm (30%), 448 nm (100%), m/z 711 [M−4Na—H₂O+5H]⁺ (9%), 533 (13%), 306 (100%); 8.33 min (39.56%), λₘₐₓ 217 nm (14%), 268 nm (20%), 228 nm (100%), m/z 711 [M−4Na−H₂O+5H]⁺ (10%), 533 (11%), 306 (100%); 8.90 min (38.09%), λₘₐₓ 223 nm (45%), 269 nm (30%), 336 nm (26%), 448 nm (100%), m/z 711 [M−4Na−H₂O+5H]⁺ (8%), 631 [M−4Na−PO₃H−H₂O+5H]⁺ (18%), 533 (20%), 306 (100%); MS (ESI-IT): m/z 816 M⁺ (55%), 772 [M−2Nz+2H]⁺ (37%), 728 [M−4Na+4H]⁺ (74%)

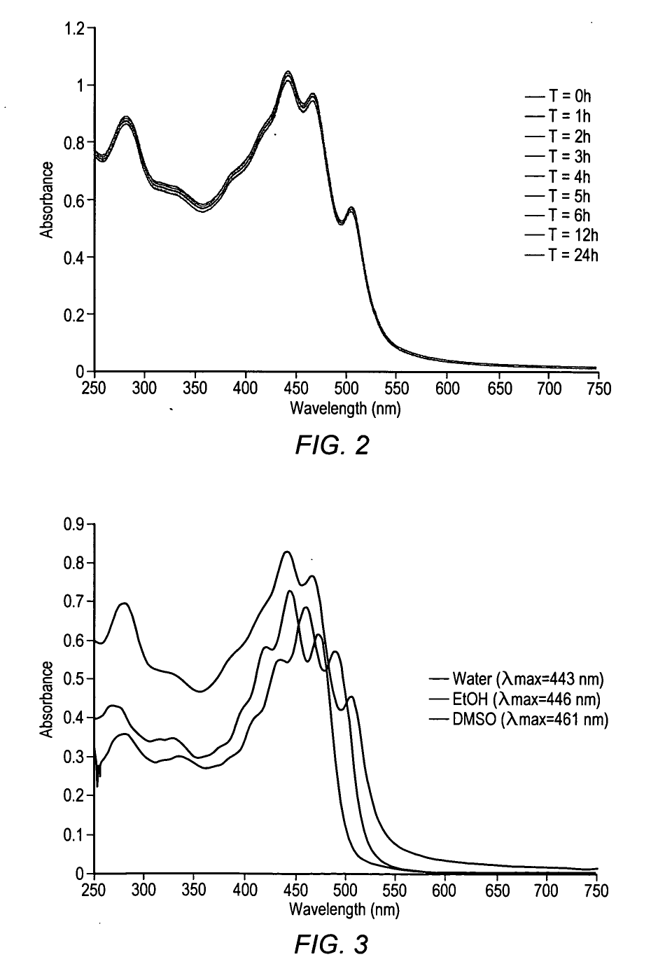

UV/Visible spectroscopy. For spectroscopic sample preparations, 3 and 9 were dissolved in the appropriate solvent to yield final concentrations of approximately 0.01 mM and 0.2 mM, respectively. The solutions were then added to a rectangular cuvette with 1 cm path length fitted with a glass stopper. The absorption spectrum was subsequently registered between 250 and 750 mn. All spectra were accumulated one time with a bandwidth of 1.0 mn at a scan speed of 370 nm/min. For the aggregation time-series measurements, spectra were obtained at baseline (immediately after solvation; time zero) and then at the same intervals up to and including 24 hours post-solvation (see FIG. 2-FIG. 7). Concentration was held constant in the ethanolic titration of the diphosphate lutein sodium salt, for which evidence of card-pack aggregation was obtained (FIG. 5-FIG. 7).

Determination of aqueous solubility/dispersibility. 30.13 mg of 3 was added to 1 mL of USP-purified water. The sample was rotated for 2 hours, then centrifuged for 5 minutes. After centrifuging, solid was visible in the bottom of the tube. A 125-μL aliquot of the solution was then diluted to 25 mL. The sample was analyzed by UV/V is spectroscopy at 436 nm, and the absorbance was compared to a standard curve compiled from 4 standards of known concentration. The concentration of the original supernatant was calculated to be 2.85 mg/mL and the absorptivity was 36.94 AU*mL/cm*mg. Slight error may have been introduced by the small size of the original aliquot.

Next, 30.80 mg of 9 was added to 1 mL of USP-purified water. The sample was rotated for 2 hours, then centrifuged for 5 minutes. After centrifuging, solid was visible in the bottom of the tube. A 125-μL aliquot of the solution was then diluted to 25 mL. The sample was analyzed by UV/V is spectroscopy at 411 nm, and the absorbance was compared to a standard curve compiled from 4 standards of known concentration. The concentration of the original supernatant was calculated to be 29.27 mg/mL and the absorptivity was 2.90 AU*mL/cm*mg. Slight error may have been introduced by the small size of the original aliquot.

Leukocyte Isolation and Preparation. Human polymorphonuclear leukocytes (PMNs) were isolated from freshly sampled venous blood of a single volunteer (S.F.L.) by Percoll density gradient centrifugation as described previously. Briefly, each 10 mL of whole blood was mixed with 0.8 mL of 0.1 M EDTA and 25 mL of saline. The diluted blood was then layered over 9 mL of Percoll at a specific density of 1.080 g/mL. After centrifugation at 400×g for 20 min at 20° C., the plasma, mononuclear cell, and Percoll layers were removed. Erythrocytes were subsequently lysed by addition of 18 mL of ice-cold water for 30 s, followed by 2 mL of 10× PIPES buffer (25 mM PIPES, 110 mM NaCl, and 5 mM KCl, titrated to pH 7.4 with NaOH). Cells were then pelleted at 4° C., the supernatant was decanted, and the procedure was repeated. After the second hypotonic cell lysis, cells were washed twice with PAG buffer [PIPES buffer containing 0.003% human serum albumin (HSA) and 0.1% glucose]. Afterward, PMNs were counted by light microscopy on a hemocytometer. The isolation yielded PMNs with a purity of >95%. The final pellet was then suspended in PAG-CM buffer (PAG buffer with 1 mM CaCl₂ and 1 mM MgCl₂).

EPR Measurements. All EPR measurements were performed using a Bruker ER 300 EPR spectrometer operating at X-band with a TM₁₁₀ cavity as previously described. The microwave frequency was measured with a Model 575 microwave counter (EIP Microwave, Inc., San Jose, Calif.). To measure superoxide anion (O\overline{}₂) generation from phorbol-ester (PMA)-stimulated PMNs, EPR spin-trapping studies were performed using the spin trap DEPMPO (Oxis, Portland, Oreg.) at 10 mM. 1×10⁶ PMNs were stimulated with PMA (1 ng/mL) and loaded into capillary tubes for EPR measurements. To determine the radical scavenging ability of 3 and 9 in aqueous and ethanolic formulations, PMNs were pre-incubated for 5 minutes with test compound, followed by PMA stimulation.

Instrument settings used in the spin-trapping experiments were as follows: modulation amplitude, 0.32 G; time constant, 0.16 s; scan time, 60 s; modulation frequency, 100 kHz; microwave power, 20 milliwatts; and microwave frequency, 9.76 GHz. The samples were placed in a quartz EPR flat cell, and spectra were recorded. The component signals in the spectra were identified and quantified as reported previously.

UV/V is Spectral Properties in Organic and Aqueous Solvents.

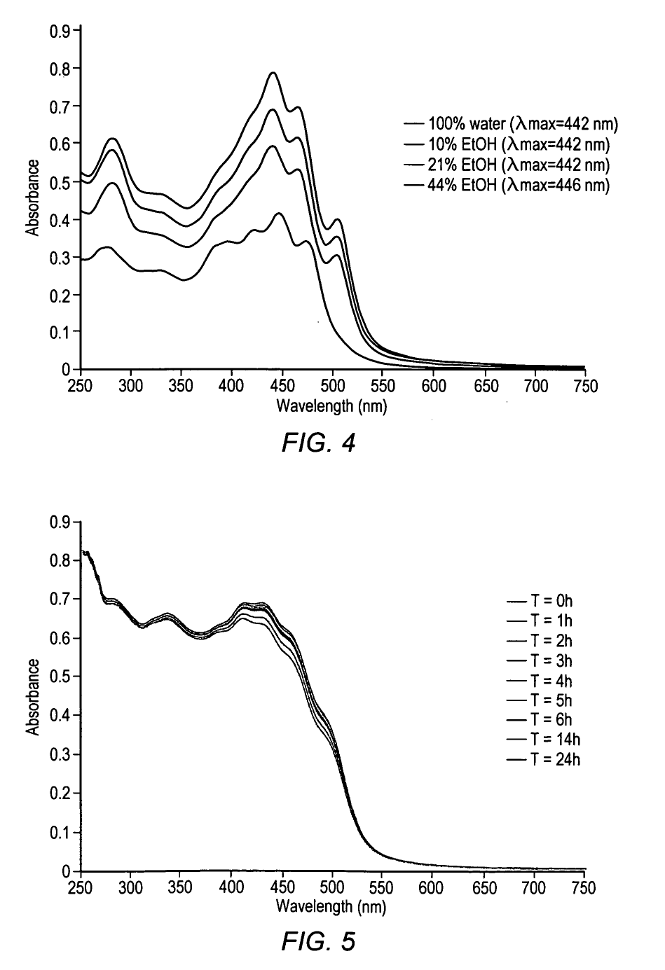

UV-V is spectral evaluation of the disuccinate lutein sodium salt is depicted in FIG. 2 -FIG. 4. FIG. 2 depicts a time series of the UV/V is absorption spectra of the disodium disuccinate derivative of natural source lutein in water. The λₘₐₓ (443 nm) obtained at time zero did not appreciably blue-shift over the course of 24 hours, vibrational fine structure was maintained (% III/II=35%), and the spectra became only slightly hypochromic (i.e. decreased in absorbance intensity) over time, indicating minimal time-dependent supramolecular assembly (aggregation) of the card-pack type during this time period. Existence of head-to-tail (J-type) aggregation in solution cannot be ruled out.

FIG. 3 depicts a UV/V is absorption spectra of the disodium disuccinate derivative of natural source lutein in water (λₘₐₓ=443 nm), ethanol (λₘₐₓ=446 nm), and DMSO (λₘₐₓ=461 nm). Spectra were obtained at time zero. A prominent cis peak is seen with a maximum at 282 nm in water. The expected bathochromic shift of the spectrum in the more polarizable solvent (DMSO) is seen (461 nm). Only a slight hypsochromic shift is seen between the spectrum in water and that in ethanol, reflecting minimal card-pack aggregation in aqueous solution. Replacement of the main visible absorption band observed in EtOH by an intense peak in the near UV region—narrow and displaying no vibrational fine structure—is not observed in the aqueous solution of this highly water-dispersible derivative, in comparison to the spectrum of pure lutein in an organic/water mixture.

FIG. 4 depicts a UV/V is absorption spectra of the disodium disuccinate derivative of natural source lutein in water (λₘₐₓ=442 nm) with increasing concentrations of ethanol. The λₘₐₓ increases to 446 nm at an EtOH concentration of 44%, at which point no further shift of the absorption maximum occurs (i.e. a molecular solution has been achieved), identical to that obtained in 100% EtOH (See FIG. 3).

UV-V is spectral evaluation of the diphosphate lutein sodium salt is depicted in FIG. 5-FIG. 7. FIG. 5 depicts a time series of the UV/V is absorption spectra of the disodium diphosphate derivative of natural source lutein in water. Loss of vibrational fine structure (spectral distribution beginning to approach unimodality) and the blue-shifted lambda max relative to the lutein chromophore in EtOH suggested that card-pack aggregation was present immediately upon solvation. The λₘₐₓ (428 nm) obtained at time zero did not appreciably blue-shift over the course of 24 hours, and the spectra became slightly more hypochromic over time (i.e. decreased in absorbance intensity), indicating additional time-dependent supramolecular assembly (aggregation) of the card-pack type during this time period. This spectrum was essentially maintained over the course of 24 hours (compare with FIG. 2, disuccinate lutein sodium salt).

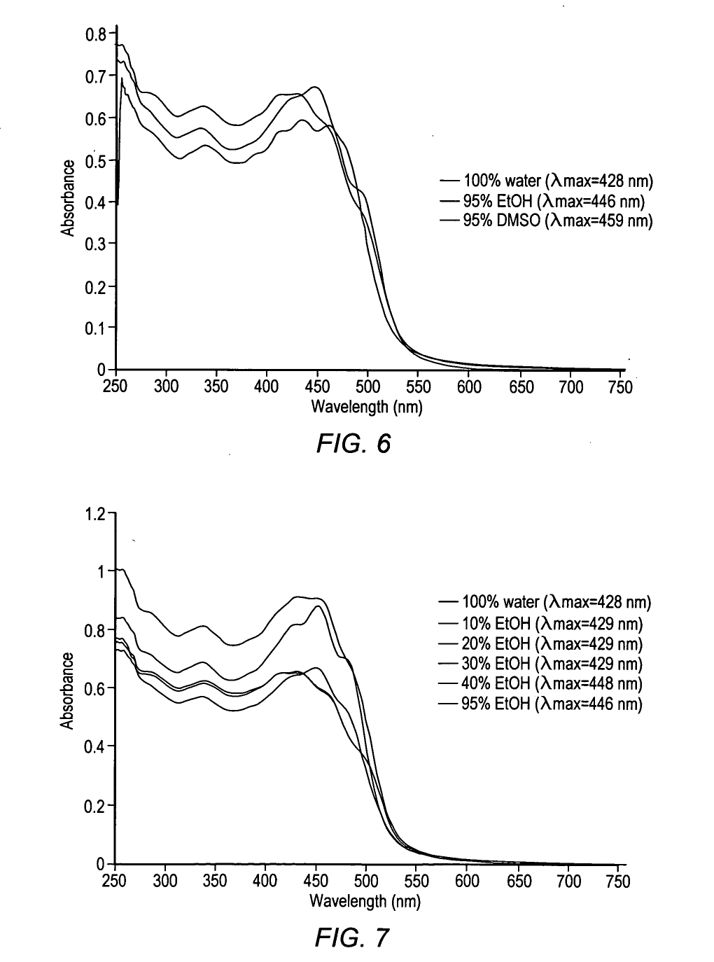

FIG. 6 depicts a UV/V is absorption spectra of the disodium diphosphate derivative of natural source lutein in 95% ethanol (λₘₐₓ=446 nm), 95% DMSO (λₘₐₓ=459 nm), and water (λₘₐₓ=428 nm). A red-shift was observed (λₘₐₓ to 446 nm), as was observed with the disuccinate derivate. Wetting of the diphosphate lutein derivative with a small amount of water was required to obtain appreciable solubility in organic solvent (e.g. EtOH and DMSO). Spectra were obtained at time zero. The expected bathochromic shift (in this case to 459 nm) of the spectrum in the more polarizable solvent (95% DMSO) is seen. Increased vibrational fme structure and red-shifting of the spectra were observed in the organic solvents.

FIG. 7 depicts a UV/V is absorption spectra of the disodium diphosphate derivative of natural source lutein in water (λₘₐₓ=428 nm) with increasing concentrations of ethanol. Concentration of the derivative was held constant for each increased concentration of EtOH in solution. The λₘₐₓ increases to 448 nm at an EtOH concentration of 40%, at which no ftirther shift of the absorption maximum occurs (i.e. a molecular solution is reached).

Direct Superoxide Anion Scavenging by EPR Spectroscopy

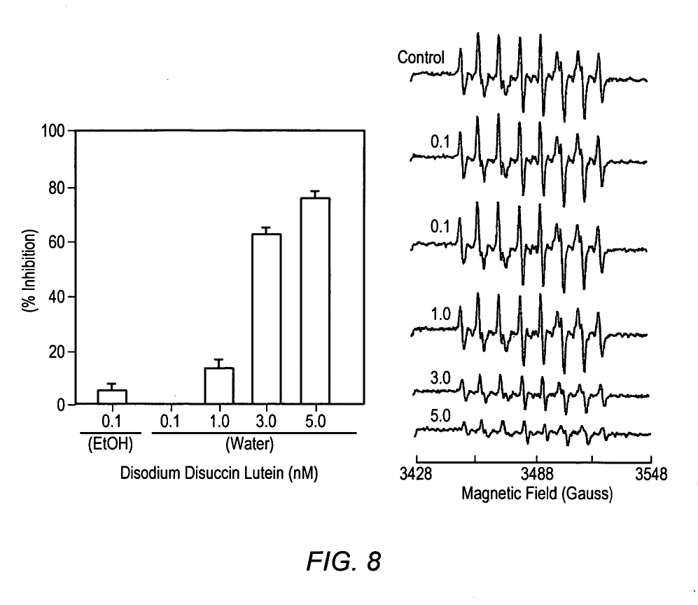

The mean percent inhibition of superoxide anion signal (±SEM) as detected by DEPMPO spin-trap by the disodium disuccinate derivative of natural source lutein (tested in water) is shown in FIG. 8. A 100 μM formulation (0.1 mM) was also tested in 40% EtOH, a concentration shown to produce a molecular (i.e. non-aggregated) solution. As the concentration of the derivative increased, inhibition of superoxide anion signal increased in a dose-dependent manner. At 5 mM, approximately ¾ (75%) of the superoxide anion signal was inhibited. No significant scavenging (0% inhibition) was observed at 0.1 mM in water. Addition of 40% EtOH to the derivative solution at 0.1 mM did not significantly increase scavenging over that provided by the EtOH vehicle alone (5% inhibition). The millimolar concentration scavenging by the derivative was accomplished in water alone, without the addition of organic co-solvent (e.g., acetone, EtOH), heat, detergents, or other additives. This data suggested that card-pack aggregation for this derivative was not occurring in aqueous solution (and thus limiting the interaction of the aggregated carotenoid derivative with aqueous superoxide anion).

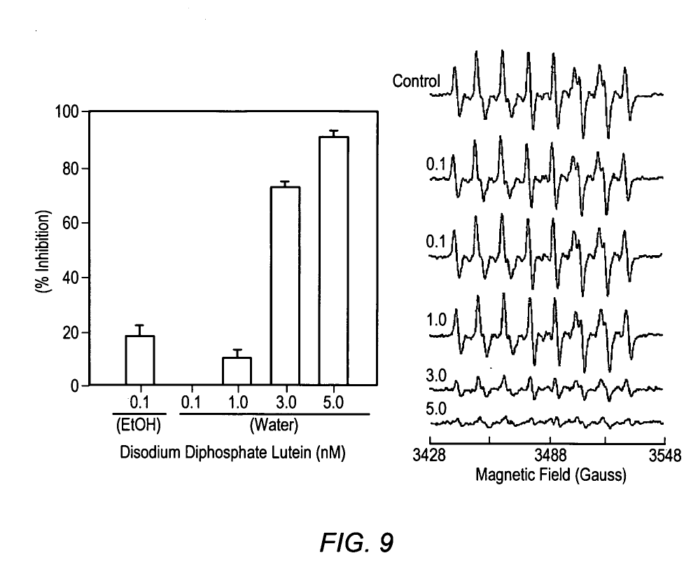

The mean percent inhibition of superoxide anion signal (±SEM) as detected by DEPMPO spin-trap by the disodium diphosphate derivative of natural source lutein (tested in water) is shown in FIG. 9. A 100 μM formulation (0.1 mM) was also tested in 40% EtOH, a concentration also shown to produce a molecular (i.e. non-aggregated) solution of this derivative. As the concentration of the derivative increased, inhibition of the superoxide anion signal increased in a dose-dependent manner. At 5 mM, slightly more than 90% of the superoxide anion signal was inhibited (versus 75% for the disuccinate lutein sodium salt). As for the disuccinate lutein sodium salt, no apparent scavenging (0% inhibition) was observed at 0.1 mM in water. However, a significant increase over background scavenging by the EtOH vehicle (5%) was observed after the addition of 40% EtOH , resulting in a mean 18% inhibition of superoxide anion signal. This suggested that disaggregation of the compound lead to an increase in scavenging ability by this derivative, pointing to slightly increased scavenging ability of molecular solutions of the more water-dispersible diphosphate derivative relative to the disuccinate derivative. Again, the millimolar concentration scavenging by the derivative was accomplished in water alone, without the addition of organic co-solvent (e.g., acetone, EtOH), heat, detergents, or other additives.

TABLE 1

| | | | | Mean | | | | | |
|---|---|---|---|---|---|---|---|---|---|
| Sample | Solvent | Concentration | N | (% inhibition) | S.D. | SEM | Min | Max | Range |
| Lutein Disuccinate | 40% | 0.1 mM | 3 | 5.0 | 4.4 | 2.5 | 0 | 8 | 8 |
| Sodium Salt | EtOH | | | | | | | | |
| Lutein Disuccinate | Water | 0.1 mM | 1 | 0.0 | ND | ND | 0 | 0 | 0 |
| Sodium Salt | | | | | | | | | |
| Lutein Disuccinate | Water | 1.0 mM | 3 | 13.0 | 5.6 | 3.2 | 8 | 19 | 11 |
| Sodium Salt | | | | | | | | | |
| Lutein Disuccinate | Water | 3.0 mM | 3 | 61.7 | 4.0 | 2.3 | 58 | 66 | 8 |
| Sodium Salt | | | | | | | | | |
| Lutein Disuccinate | Water | 5.0 mM | 3 | 74.7 | 4.5 | 2.6 | 70 | 79 | 9 |
| Sodium Salt | | | | | | | | | |

Descriptive statistics of mean % inhibition of superoxide anion signal for aqueous and ethanolic (40%) formulations of disodium disuccinate derivatives of natural source lutein tested in the current study. Sample sizes of 3 were evaluated for each formulation, with the exception of natural source lutein in 40% EtOH stock solution (N=1). Mean % inhibition did not increase over background levels until sample concentration reached 1 mM in water; likewise, addition of 40% EtOH at the 0.1 mM concentration did not increase scavenging over background levels attributable to the EtOH vehicle (mean=5% inhibition).

TABLE 2

| | | | | Mean | | | | | |
|---|---|---|---|---|---|---|---|---|---|
| Sample | Solvent | Concentration | N | (% inhibition) | S.D. | SEM | Min | Max | Range |
| Lutein Diphosphate | 40% | 0.1 mM | 3 | 18.0 | 7.0 | 4.0 | 11 | 25 | 14 |
| Sodium Salt | EtOH | | | | | | | | |
| Lutein Diphosphate | Water | 0.1 mM | 1 | 0.0 | ND | ND | 0 | 0 | 0 |
| Sodium Salt | | | | | | | | | |
| Lutein Diphosphate | Water | 1.0 mM | 3 | 9.3 | 3.5 | 2.0 | 6 | 13 | 7 |
| Sodium Salt | | | | | | | | | |
| Lutein Diphosphate | Water | 3.0 mM | 3 | 72.3 | 3.1 | 1.8 | 69 | 75 | 6 |
| Sodium Salt | | | | | | | | | |
| Lutein Diphosphate | Water | 5.0 mM | 3 | 91.0 | 2.6 | 1.5 | 88 | 93 | 5 |
| Sodium Salt | | | | | | | | | |

Lutein

Descriptive statistics of mean % inhibition of superoxide anion signal for aqueous and ethanolic (40%) formulations of disodium diphosphate derivatives of natural source lutein tested in the current study. Sample sizes of 3 were evaluated for each formulation, with the exception of lutein diphosphate in water at 100 μM (0.1 mM) where N=1. Mean % inhibition of superoxide anion signal increased in a dose-dependent manner as the concentration of lutein diphosphate was increased in the test assay. At 100 μM in water, no inhibition of scavenging was seen. The molecular solution in 40% EtOH (mean % inhibition=18%) was increased above background scavenging (5%) by the ethanolic vehicle, suggesting that disaggregation increased scavenging at that concentration. Slightly increased scavenging (on a molar basis) may have been obtained with the diphosphate derivative in comparison to disuccinate derivative (see Table 1 and FIG. 8).

In the current study, facile preparations of the disodium disuccinate and tetrasodium phosphate esters of natural source (RRR) lutein are described. These asymmetric C40 carotenoid derivatives exhibited aqueous dispersibility of 2.85 and 29.27 mg/mL, respectively. Evidence for both card-pack (H-type) and head-to-tail (J-type) supramolecular assembly was obtained with UV-V is spectroscopy for the aqueous solutions of these compounds. Electronic paramagnetic spectroscopy of direct aqueous superoxide scavenging by these derivatives demonstrated nearly identical dose-dependent scavenging profiles, with slightly increased scavenging noted for the diphosphate derivative. In each case, scavenging in the millimolar range was observed. These results show that as parenteral soft drugs with aqueous radical scavenging activity, both compounds are useful in those clinical applications in which rapid and/or intravenous delivery is desired for the desired therapeutic effect(s).

### EXPERIMENTAL EXAMPLES

Materials and Methods:

Cardax™ (‘racemic’ disodium disuccinate astaxanthin; “rac-dAST”) was synthesized from crystalline astaxanthin [3S,3′S, 3R,3′S, 3R,3′R (1:2:1)], a statistical mixture of stereoisomers obtained commercially (Buckton-Scott, India; Frey et al. 2004). This material was utilized for oral gavage studies in mice [purity>97.0% by HPLC, as area under the curve (AUC)].

The individual astaxanthin stereoisomers were also separated by HPLC as dicamphanate esters and then saponified to non-esterified astaxanthin, allowing for the synthesis of the meso-disodium disuccinate astaxanthin derivative (meso-dAST) for testing in the current study (Frey et al. 2004). The all-trans (all-E) form of the meso stereoisomer used was a linear, rigid molecule (bolaamphiphile) owing to the lack of cis (or Z) configuration(s) in the polyene chain of the spacer material (FIG. 2; Foss et al. 2005). The disodium disuccinate derivative of synthetic meso-astaxanthin was successfully synthesized at >99% purity by HPLC (as AUC).

Five hundred (500) U of human recombinant 5-lipoxygenase (5-LOX; Product No. 437996, Lot No. B60857; in 100 mM Tris containing 5 mM EDTA, pH 8.0; specific activity 18.48 units/mg protein) was obtained from Calbiochem (San Diego, Calif., USA) and was used without modification. Tris HCl buffer (0.1 M, pH 8.0) and spectroscopy grade dimethyl sulfoxide (DMSO, Scharlau Chemie S. A., Barcelona, Spain) were used for the in vitro studies.

Oral Administration of Test Compound to Mice

The methods of Showalter et al. (2004) were used, with slight modifications, to prepare the emulsion vehicle and dose-formulations for oral gavage to individual mice:

Emulsion Vehicle and Dose Formulation Preparation

An oil/water emulsion was prepared as follows. Soybean lecithin (500 mg, Type IV-S, Sigma-Aldrich Co., St. Louis, Mo.; catalog number P3644) was added to 10.0 mL of a 9:1 mixture of filtered (0.2 micron Millipore®) water and olive oil (2.5 mL, Bertolli USA, Inc., Secaucus, N.J.). This mixture was vortexed intermittently for approximately 30 min until the suspension was uniform. This primary emulsion was then mixed with additional water and olive oil in the proportion 2:2:1 (primary emulsion:water:oil). This final emulsion was again vortexed at room temperature, producing a uniform, thick, cloudy yellow suspension with a final proportion of 3.2 parts water to 1 part oil with 20 mg/mL lecithin. Emulsion material was stored either at room temperature for short periods, or refrigerated at 4° C. for several weeks.

Cardax™ dose formulations were then prepared as follows. The emulsion was vortexed and crystalline disodium disuccinate astaxanthin (50 mg/mL) was added. The dose formulation was again vortexed, and the compound readily entered into a uniform suspension at this concentration, allowing for dosing at 500 mg/kg by oral gavage in the mice.

Oral Gavage Dosing of C57BL/6 Mice

Male C57BL/6 mice, 8 to 12 weeks old and approximately 25 to 30 g, were housed in cages (5 mice/cage) and fed standard mouse chow (Prolab 2500, Purina, St. Louis, Mo.) and water ad libitum for at least five days prior to the start of the experiment. Mice were then fasted overnight with free access to water, and emulsion containing Cardax™ was given by oral gavage at 500 mg/kg body weight in a single dose using a 20 ga straight gavage needle (Popper, New Hyde Park, N.Y.). Typical gavage volumes were ˜25 μL per mouse. One hour after administration of the emulsion, food and water were restored to all animals. Oral gavage was provided once per day for 7 days prior to the peritoneal lavage collection for oxidative stress marker analysis on day 8.

Measurement of Oxidative Stress Markers

Total levels of specific fatty acid and protein oxidation products were quantified by HPLC with on-line electrospray ionization tandem mass spectrometry (LC/ESI/MS/MS) using methods recently described (Zhang et al. 2002). Briefly, following addition of known amounts of heavy isotope labeled internal standards, total fatty acids of lipids in both plasma and peritoneal lavage fluids were first released by base saponification. Fatty acids were then extracted into organic solvents, dried under inert atmosphere (nitrogen or argon), resuspended in mobile phase, and then injected and analyzed on-line by LC/ESI/MS/MS using characteristic parent→daughter ions for multiple specific oxidation products of arachidonic acid (AA) and linoleic acid (LA). Following integration of relevant peaks and their appropriate corresponding internal standards, levels of each analyte were calculated and presented. Results are expressed both in absolute terms (molarity, M) and relative to the level of their precursor amino acids (arachidonate for HETEs, EETs, F2-isoprostanes and oxoEETs; and linoleate for HODEs and oxoODEs).

Preparation of Stock Solution of Meso-dAST for Spectroscopic Experiments

0.27 mg of meso-dAST (M_{w}=840.95) was dissolved in 0.64 mL DMSO to yield a 5×10⁻⁴ M stock solution.

Circular Dichroism (CD) and UV/V is Absorption Spectroscopy Measurements

CD and UV/V is spectra were recorded on a Jasco J-715 spectropolarimeter at 37±0.2° C. in a rectangular cuvette with 1 cm pathlength. Temperature control was provided by a Peltier thermostat equipped with magnetic stirring. All spectra were accumulated three times with a bandwidth of 1.0 nm and a resolution of 0.5 nm at a scan speed of 100 nm/min. CD spectra were recorded and displayed as ‘Θ’ (ellipticity) in units of millidegrees (mdeg). Induced CD spectra resulting from the interaction of the meso-dAST with 5-LO were obtained by subtracting the CD spectrum of the protein from that of the complex.

CD/UV/V is Titration of 5-LOX with Meso-dAST in pH 8.0 Tris HCl Buffer Solution at 37° C.

Two hundred (200) μL of the 5-LO stock solution was mixed with 1.8 mL buffer solution in a cuvette with 1 cm optical pathlength, and then small amounts of the ligand stock solution (c=5.0×10⁻⁴ M) were added with an automatic pipette in 10 μL aliquots. The molar concentration of the enzyme (M_{w}=78,000) in the sample solution was calculated to be 4.5×10⁻⁵ M on the basis of the specific activity provided by the manufacturer (see above).

Mathematical Analysis of the CD Titration Data

The analysis of the CD titration data was based on the following approach. At the lowest enzyme/carotenoid derivative ratios, only one carotenoid molecule binds to the 5-LOX enzyme with an association constant of K₁:
E+L□EL

where ‘E’ denotes 5-LO, ‘L’ denotes the meso-dAST ligand molecule, and ‘EL’ represents the 1:1 complex of the enzyme and the carotenoid derivative. With increased ligand concentrations, a second carotenoid molecule begins to bind to 5-LO in close proximity to the first ligand, forming a right-handed chiral molecular arrangement. This complexation exhibits an association constant K₂:
EL+L□ELL

where ‘ELL’ denotes the 1:2 enzyme-carotenoid derivative complex. Thus, an equilibrium existed between the two types of enzyme-carotenoid derivative complexation, which was shifted to the ‘ELL’ species at elevated ligand concentrations. Due to the fact that only 1:2 complexes possess excitonic signal, the CD titration curve was sigmoid. Association constant determination from the CD titration data measured at 449.5 nm was carried out by a Fortran 77 program written in our laboratory [Hazai et al, JCAMD, in press].

Molecular Modeling Calculations of the Meso-dAST:5-LO Interaction

Geometric optimization of the meso-dAST:5-LOX complexation was carried out with the Sybyl 6.6 program (Tripos Inc., St. Louis, Mo.) using the MMFF94 force field and applying the Powell conjugate gradient method. The Autodock 3.0 program package was used for mapping the energetically most favorable binding of meso-dAST to 5-LO. The X-ray crystallographic structure of the mammalian 5-LO enzyme has not yet been determined. Therefore, the three-dimensional coordinates of the rabbit reticulocyte 15-lipoxygenase (rabbit 15-LO) was used as a surrogate (PDB entry 1LO). Gasteiger-Huckel partial charges were applied both for the ligand and the protein. Solvation parameters were added to the protein coordinate files, and the ligand torsions were defined using the ‘Addsol’ and ‘Autotors’ program utilities, respectively. The atomic affinity grids were prepared with 0.375 Å spacing by the Autogrid program for the whole protein target. Random starting positions, orientations and torsions (for flexible bonds) were used for the ligand. Each docking run consisted of 100 cycles.

Statistical Analyses

Statistical analyses were performed with the NCSS statistical package (Kaysville, Utah). Student's t test was used for evaluation of group means when data was normally distributed; Wilcoxon's rank sum test was used for non-normally distributed data. Data are reported as mean±standard error of the mean (mean ±SEM). In Table 3, data significant at the P<0.05 level are shaded; non-parametric testing is identified as “NP”.

Results

Electrospray ionization tandem mass spectrometry was used to simultaneously quantify individual molecular species of HETEs, HPETEs, the prostaglandin PGF_{2α}, F₂-isoprostanoids, hydroxy- and hydroperoxy-octadecadienoic acids (H(P)ODEs), and their precursors, arachidonic acid (AA) and linoleic acid (LA). Additionally, the molar ratios of o-tyrosine (oY/F). were measured.

Oxidative Stress Markers

Forty-two (42) individual parameters were evaluated, at 5 separate time points, for each of the Cardax™-treated and placebo-treated mice supernatant samples (Table 1), yielding a matrix of 210 individual cells per treatment grouping. For all comparisons that reached statistical significance at P<0.05, the individual cell was shaded in gray.

At baseline, all comparisons among individual oxidative and cellular parameters was non-significant except for two cases (8-HETE ratio and CLY/Y); in each of the subsequent time points, no significant differences between groups was identified for these 2 parameters. Hence, at baseline, the groups were very similar in terms of the assayed markers.

Arachidonic acid (AA) and linoleic acid (LA) were significantly elevated in the Cardax™-treated group at 16 h thioglycollate and at 72 h thioglycollate/4 h zymosan, indicating a sparing effect for these 2 oxidative substrates at these timepoints.

The following parameters exhibited a significant decrease (P <0.05) following 7-day oral treatment with Cardax™:
PGF_{2α} (ratio) at 72 h thioglyocollate/4 h zymosan
11-HETE (ratio) at 72 h thioglycollate/4 h zymosan
12-HETE (ratio) at 16 h thioglycollate
5-HETE (ratio) at 72 h thioglycollate/4 h zymosan
8-iso-F_{2α} (pmol) at 16 h thioglycollate/4 h zymosan
8-iso-F_{2α} (ratio) at 16 h thioglycollate/4 h zymosan
9-HODE (ratio) at 72 h thioglycollate/4 h zymosan
5-oxo-EET (ratio) at 72 h thioglycollate/4 h zymosan
oY/F (mmol/mol) at 16 h thioglycollate/4 h zymosan

In addition, the following parameters exhibited a significant increase (P<0.05) following 7-day oral treatment with Cardax™:
12-HETE (pmol) at 72 h thioglycollate/4 h zymosan
15-HETE (pmol) at 72 h thioglycollate/4 h zymosan
8-HETE (pmol) at 72 h thioglycollate/4 h zymosan
5,6-EET (pmol) at 16 h thioglycollate
DiY/Y at 16 h thioglycollate
NO2/Y at 16 h thioglycollate

For the oxidative marker elevations of 8-, 12-, and 15-HETE, as well as 5,6-EET, these significant increases disappeared when normalized to the concentration of the starting substrate (AA) in each case.

Significant differences in 2 cellular parametes (LYMPHS, RBC) identified at the 16 h thioglycollate time point did not persist at the 72 h thioglycollate time point.

Table 3 summarizes the statistical analyses of in vivo lipid oxidation molecular fingerprint data obtained with the experimentally induced peritonitis model in mice treated with ddAst (Cardax™) in greater detail.

UV/V is and CD Spectroscopic Evaluation of Meso-dAST and 5-LOX

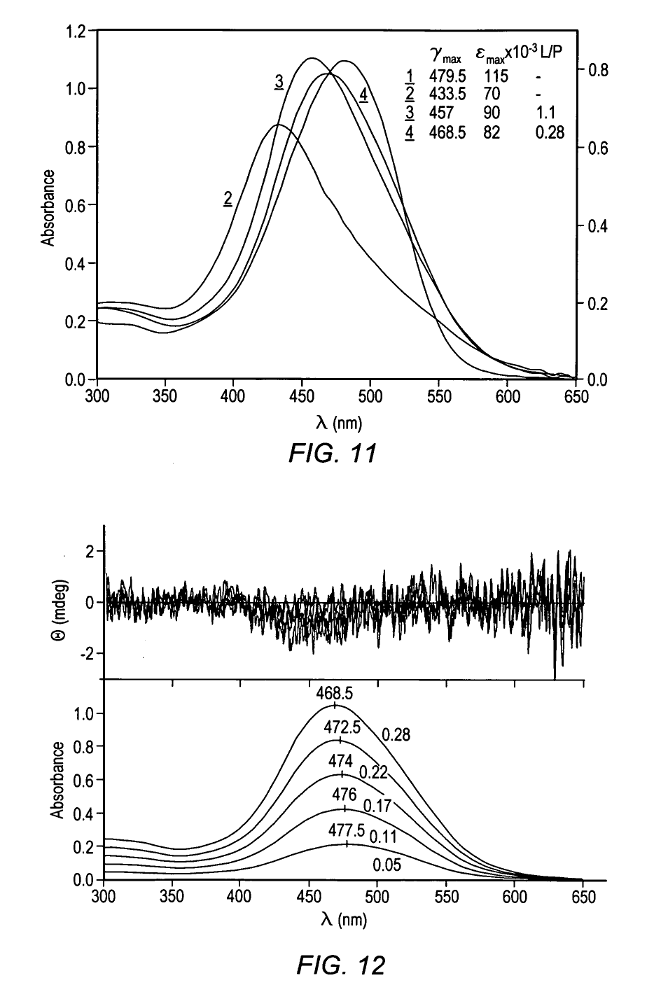

The UV/V is absorption spectra of meso-dAST in ethanol (EtOH) and Tris buffer, in the presence and absence of 5-LO, are shown in FIG. 11. In Tris buffer alone, the meso-dAST spectrum is blue-shifted and hypochromic relative to that in EtOH, or in the presence of 5-LO in either buffer.

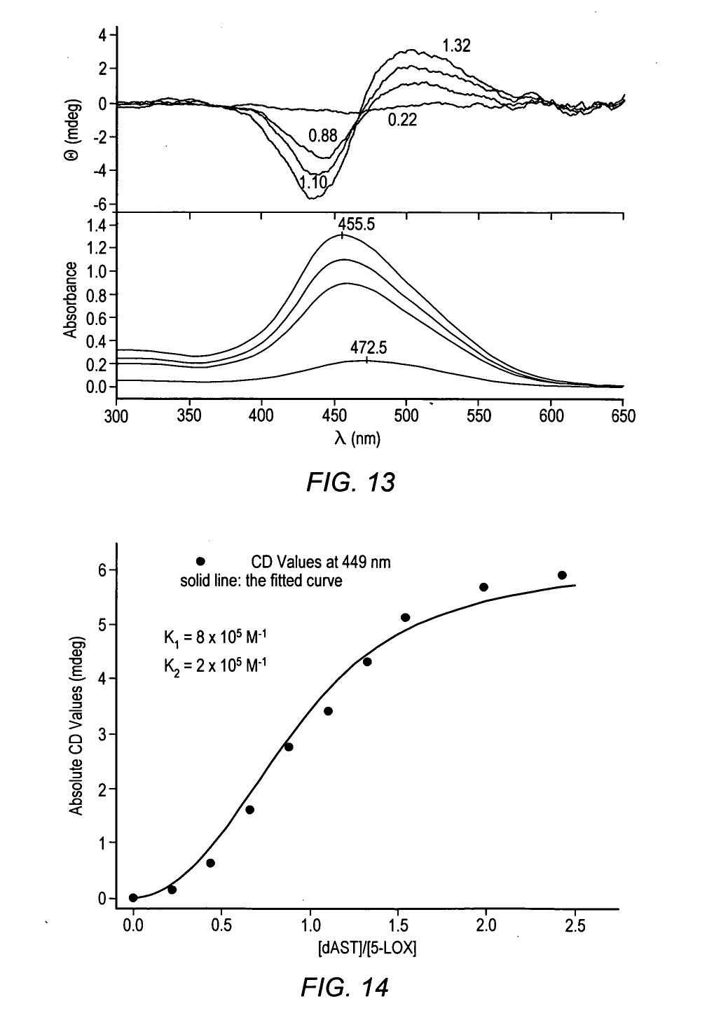

The titration of a constant concentration of 5-LO (4.5×10⁻⁵ M) in buffer solution with differing concentrations of meso-dAST is shown in FIGS. 12 and 13. As the concentration of the carotenoid ligand increased, the UV/V is spectrum shifted slightly to shorter wavelengths; however, no induced CD activity was detected at L/P ratios below 0.3 (FIG. 4). As the L/P ratio was increased above 0.5, long-wavelength positive and short-wavelength negative CD bands appeared (FIG. 13).

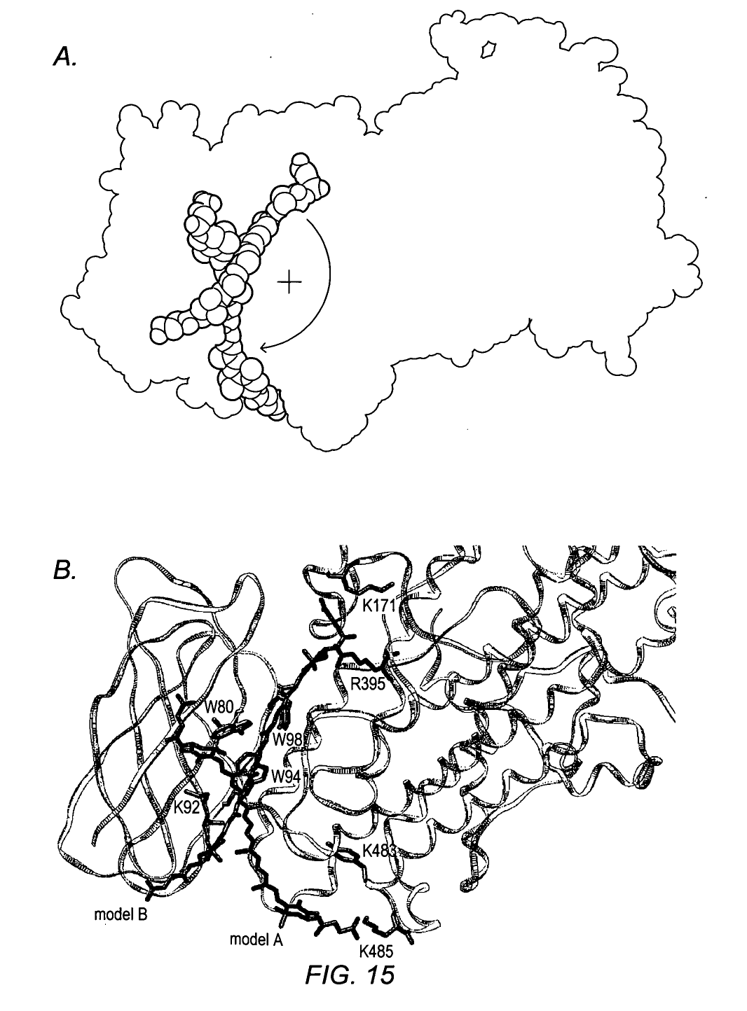

The association constant for the ligand/protein binding was determined after fitting the induced circular dichroism data obtained during titration of 5-LO with meso-dAST (FIG. 14). The obtained “best fit” curve was sigmoidal in shape. Computational docking of the meso-dAST molecule with the published XRC data for mammalian 15-LO is shown in FIG. 15.

Molecular solutions of meso-dAST can be prepared easily using organic solvents. The visible absorption spectrum of meso-dAST in ethanol (EtOH) exhibits a typical C40 carotenoid patten (FIG. 11), reflecting the strongly allowed π-π*-type transition with unresolved vibrational bands. The absence of vibrational fine structure is consistent with the absorption spectra of other carotenoids (and in particular C40 ketocarotenoids such as astaxanthin) in which the conjugation extends to various types of terminal rings (Frank et al. 2000; Zigmantas et al. 2004). Despite the presence of the ionizable carboxylic groups which substantially enhance the water solubility of all isomeric forms of dAST, the molecule preserves its tendency to form self-aggregates in aqueous environments (Zsila et al. 2003). Accordingly, a large blue shift (479.5 nm→433.5 nm), bandwidth narrowing, and absorbance intensity loss (hypochromism) were measured in alkaline buffer solution (FIG. 11), indicating the formation of the so called “card-pack” or H-type aggregates in this solution. In these aggregates, the polyene chains are stacked on each other in a nearly parallel arrangement (Bikádi et al. 2002).

Due to the tight packing within the supramolecular assemblies observed in solutions of the carotenoid derivative alone, the electronic transition moments of the neighbouring polyene chromophores interact with each other resulting in the observed spectral changes (Zsila et al. 2003; Zsila et al. 2001). However, the presence of 5-LO in the buffer solution appears to hamper the self-aggregation of meso-dAST molecules. As shown by the absorption spectra in FIG. 3, the enzyme significantly decreased the extent of the blue shift of the main peak. For example, at an L/P ratio of 0.28 (curve 4 in FIG. 11), the absorption spectrum of the meso-dAST: 5-LO solution only differed slightly from the absorption spectrum of meso-dAST alone measured in EtOH. Additionally, the molar absorption coefficients (6) calculated on the basis of the total carotenoid concentration of the samples were also significantly higher in the presence of 5-LOX when compared with the values obtained for meso-dAST alone in buffer solution. Overall, these data suggested a carotenoid-enzyme binding interaction which prevented the aqueous aggregation of the ligand molecules.

Due to the mutual cancellation effects of the opposite absolute stereochemical configurations of its 3 and 3′ chiral centers, meso-dAST shows no CD activity either in organic or aqueous solutions (data not shown). However, as demonstrated previously with meso-dAST—human serum albumin (HSA) binding (Zsila et al. 2003), the carotenoid molecule can exhibit induced CD activity upon interaction with a chiral protein environment. CD titration measurements were conducted at two different enzyme concentrations. First, meso-dAST was added consecutively to 4.5×10⁻⁵ M solutions of 5-LOX dissolved in Tris HCl buffer solution (pH 8.0). As the concentration of the meso-dAST increased, the λₘₐₓ of the absorption spectrum was shifted slightly to shorter wavelengths (by 9 nm); however, no net CD activity was observed during the entire titration range (FIG. 12). Notably, the value of the L/P ratio did not exceed 0.3.

In the second set of experiments, the meso-dAST:5-LOX ratio was increased from 0.2 to 2.4. Above an L/P ratio of 0.5, a long-wavelength positive and a short-wavelength negative CD band pair developed in the visible absorption region (FIG. 13). The intensities of the CD band pair increased with the increasing concentration of meso-dAST, but showed no further amplification upon reaching a 1:2 5-LOX: meso-dAST ratio (FIG. 14). Such an oppositely-signed (bisignate) CD band pair is typical for chiral exciton coupling, when either two identical or differing chromophores set up a chiral spatial arrangement and their electronic transitions interact with each other. This spatial arrangement gives rise to a CD couplet, and the sign order is determined by the absolute sense of the intermolecular angle between the two transition moments (Harada and Nakanishi 1972; Lightner and Gurst 2000). When the long-wavelength CD band is positive, the exciton chirality rule predicts a positive angle between the interacting transition moments. In our case, the long axis of the meso-dAST molecule corresponds to the orientation of the π-π* electronic transition moment. Therefore, the CD results suggested that two carotenoid molecules bound adjacently to the enzyme arranged in such a manner that their long axes formed a positive (clockwise) intermolecular overlay angle. At low L/P ratios, only the first binding site was occupied, and thus exciton-type CD activity could not be observed (FIG. 12).

As previously demonstrated, HSA binding of a single meso-dAST molecule produced induced, but non-excitonic type CD bands due to the formation of a helical chiral conformation of the terminal ring and the backbone double bond system upon interaction with the asymmetric protein binding site (Zsila et al. 2003; Zsila et al. 2004). In the case of albumin, the immediate binding site residues determined the helicity of the conjugated π-system of the ligand molecule via non-covalent chemical interactions. The lack of any extrinsic Cotton effects (CE) at low L/P ratios of the meso-dAST: 5-LOX mixture suggested that the carotenoid molecule bound at the surface of the enzyme. The probable binding site would be a shallow cleft or a channel on the enzyme surface, whose residues would not tightly encompass the ligand; therefore, the conformational motions of the carotenoid end groups would be only slightly restricted. This “superficial binding” concept is further supported by the lack of a bathochromic shift of the main absoprtion peak of the mixture (relative to the ethanolic spectrum), even at high enzyme concentrations (FIG. 12).

Protein-induced red-shifts of the absorption band of carotenoids bound in carotenoprotein complexes are well known and described (Zsila et al. 2003; Zsila et al. 2004; Jouni and Wells 1996). The high polarizability of the protein matrix decreases the π-π* excitation energy of the polyene chain when surrounded by hydrophobic residues, and shifts the λₘₐₓ to longer wavelengths (longer than that measured in EtOH). However, if the carotenoid binds in a superficial, solvent-exposed pocket of the protein, this red-shifting mechanism does not operate. This is attributed to the direct contact between the conjugated backbone of the carotenoid and water molecules in solution.

The induced CD values plotted as a function of the meso-dAST:5-LOX ratios are displayed in FIG. 14. The sigmoidal curves can be modeled by assuming that 5-LOX can bind two carotenoid molecules at two binding sites in close proximity, and that the excitonic signal is directly proportional to the concentration of the enzyme-carotenoid complex. The slow increase of the CD values between 0 and 0.5 ligand/protein ratio indicates that in this region only one ligand is bound per protein. The sudden increase in CD value with an inflexion at the 1:1 L/P ratio indicates the binding of the second meso-dAST to the protein, with saturation at the 1:2 L/P ratio. Our calculations suggest an association constant of 8×10⁵ M⁻¹ for the first ligand, and a 4X-weaker affinity for the second meso-dAST molecule (FIG. 14).

Molecular Modeling Study of the Meso-dAST:5-Lipoxygenase Interaction

As the three-dimensional structure of 5-LOX has not been resolved yet, the known homologous structure of the rabbit reticulocyte 15-lipoxygenase (15-LOX) was used in our docking calculations. The sequence identity between the two enzymes is ˜40%; direct comparison of the two sequences (rabbit and human) was performed using BioEdit software, and sequences were obtained from the PIR database at Georgetown University. Meso-dAST was docked to the surface of 15-LOX in each of the docking runs. The two “best-energy” docking results are shown in FIG. 15. Both meso-dAST molecules were docked into a surface pocket with spatial geometry capable of binding long, straight ligands. Model ‘A’ is more buried by the protein residues than model ‘B’ (see FIG. 15), but in both cases the polyene chains are largely accessible to solvent molecules. Due to the stereochemistry of the binding pocket, the relative orientations of the carotenoid molecules define a positive intermolecular overlay angle. This finding is in harmony with the the positive-twist orientation between the enzyme-bound meso-dAST molecules that was reflected by the clear positive exciton couplet measured experimentally. The long, hydrophobic polyene chains are in contact with several apolar residues, mostly alanines, leucines and isoleucines. Additionally, π-π stacking can occur between the conjugated backbones and adjacent aromatic residues of the binding site: W80 and Y483 appear to participate in such interaction in the case of model ‘A’, while W94 and Y98 form similar contacts in model ‘B’ (FIG. 15B). Beside these hydrophobic interactions, salt bridges between the carboxyl groups of meso-dAST and basic residues (K92, K171, R395, K485) might be also involved in the binding interaction, further strengthening the overall L/P binding (FIG. 15B).

TABLE 3
Means (± standard errors of the mean, SEM) for each oxidative/cellular parameter measured
at each of the 5 time points in the current study. Results of statistical testing (parametric,
Student's t-test; non-parametric, Wilcoxon rank-sum test) are shown; individual comparisons with
P ≦ 0.05 are shaded. An N of 6 to 8 animals was evaluated for each dosing group at each time point.

| | 0 hr | | | 16 hr | | |
|---|---|---|---|---|---|---|
| | baseline | | | thioglycollate | | |
| | Cardax | NS | | Cardax | NS | |
| | Mean ± S.D. | Mean ± S.D. | P-Value | Mean ± S.D. | Mean ± S.D. | P-Value |
| AA (pmol) | 587.5 ± 516.8 | 524.4 ± 427.8 | 1000 NP | 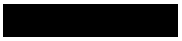 | 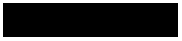 | 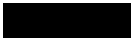 |
| LA (pmol) | 8239 ± 6058 | 8939 ± 5859 | 0.843 | 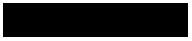 | 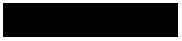 | 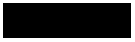 |
| PGF_{2α}(pmol) | 0.036 ± 0.081 | 0.095 ± 0.143 | 0.158 NP | 0.037 ± 0.029 | 0.020 ± 0.017 | 0.172 |
| PGF_{2α}(ratio) | 0.033 ± 0.056 | 0.159 ± 0.123 | 0.081 NP | 0.034 ± 0.036 | 0.034 ± 0.034 | 0.986 |
| 11-HETE (pmol) | 0.121 ± 0.093 | 0.091 ± 0.087 | 0.471 NP | 0.434 ± 0.393 | 0.711 ± 1.451 | 0.372 NP |
| 11-HETE (ratio) | 0.219 ± 0.058 | 0.176 ± 0.074 | 0.290 | 0.239 ± 0.132 | 0.815 ± 1.538 | 0.793 NP |
| 12-HETE (pmol) | 8.931 ± 7.471 | 4.207 ± 1.596 | 0.161 | 10.17 ± 6.720 | 14.39 ± 12.86 | 0.425 |
| 12-HETE (ratio) | 16.20 ± 7.290 | 11.25 ± 6.636 | 0.246 | 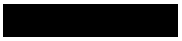 | 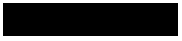 | 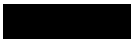 |
| 13-HODE (pmol) | 26.62 ± 22.16 | 15.70 ± 5.869 | 0.271 | 18.84 ± 13.15 | 18.69 ± 12.69 | 0.982 |
| 13-HODE (ratio) | 2.976 ± 1.375 | 2.300 ± 1.235 | 0.391 | 0.905 ± 0.326 | 2.660 ± 2.762 | 0.096 |
| 15-HETE (pmol) | 5.374 ± 5.429 | 2.029 ± 1.049 | 0.378 NP | 3.649 ± 2.385 | 7.251 ± 11.103 | 0.958 NP |
| 15-HETE (ratio) | 8.632 ± 4.144 | 5.424 ± 3.998 | 0.202 | 2.096 ± 0.541 | 8.932 ± 11.703 | 0.318 NP |
| 5,6-EET (pmol) | 0.092 ± 0.093 | 0.085 ± 0.105 | 0.936 NP | 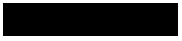 | 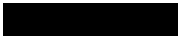 | 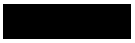 |
| 5,6-EET (ratio) | 1.148 ± 0.071 | 0.144 ± 0.059 | 0.905 | 0.115 ± 0.049 | 0.073 ± 0.040 | 0.081 |
| 5-HETE (pmol) | 0.096 ± 0.144 | 0.058 ± 0.088 | 0.471 NP | 0.334 ± 0.261 | 0.548 ± 1.132 | 0.318 NP |
| 5-HETE (ratio) | 0.129 ± 0.060 | 0.092 ± 0.057 | 0.302 | 0.218 ± 0.148 | 0.617 ± 1.203 | 0.875 NP |
| 8-HETE (pmol) | 0.386 ± 0.401 | 0.141 ± 0.082 | 0.230 NP | 0.747 ± 0.595 | 0.875 ± 1.180 | 0.495 NP |
| 8-HETE (ratio) | 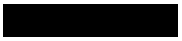 | 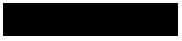 | 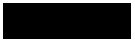 | 0.392 ± 0.155 | 1.082 ± 1.238 | 0.270 NP |
| 9-HETE (pmol) | 0.510 ± 0.475 | 0.290 ± 0.128 | 0.810 NP | 0.826 ± 0.622 | 1.375 ± 2.052 | 0.958 NP |
| 9-HETE (ratio) | 0.879 ± 0.355 | 0.711 ± 0.331 | 0.416 | 0.476 ± 0.247 | 1.716 ± 2.188 | 0.227 NP |
| 9-HODE (pmol) | 1.791 ± 1.461 | 1.869 ± 1.151 | 0.920 | 5.367 ± 3.820 | 2.783 ± 2.210 | 0.120 |
| 9-HODE (ratio) | 0.212 ± 0.147 | 0.229 ± 0.079 | 0.298 NP | 0.292 ± 0.167 | 0.419 ± 0.541 | 0.564 NP |
| 8-iso-F_{2α}(pmol) | 0.156 ± 0.134 | 0.089 ± 0.109 | 0.354 | 0.052 ± 0.046 | 0.075 ± 0.060 | 0.874 NP |
| 8-iso-F_{2α}(ratio) | 0.307 ± 0.284 | 0.163 ± 0.142 | 0.292 | 0.044 ± 0.049 | 0.117 ± 0.131 | 0.065 NP |
| 5-oxo-ETE (pmol) | 0.020 ± 0.035 | 0.115 ± 0.257 | 0.789 NP | 0.128 ± 0.132 | 0.045 ± 0.040 | 0.113 |
| 5-oxo-ETE (ratio) | 0.033 ± 0.057 | 0.200 ± 0.403 | 0.532 NP | 0.056 ± 0.043 | 0.056 ± 0.037 | 0.998 |
| ALV MAC % | 0 ± 0 | 0 ± 0 | N/A | 3.000 ± 1.000 | 13.50 ± 15.97 | 0.316 |
| EOSIN % | 0 ± 0 | 0 ± 0 | N/A | 6.000 ± 4.359 | 2.750 ± 1.500 | 0.199 NP |
| LYMPHS % | 0 ± 0 | 0 ± 0 | N/A | 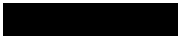 | 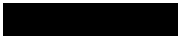 | 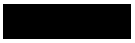 |
| MONOS % | 0 ± 0 | 0 ± 0 | N/A | 39.67 ± 14.57 | 23.75 ± 9.605 | 0.139 |
| NEUTRO % | 0 ± 0 | 0 ± 0 | N/A | 25.33 ± 17.90 | 10.50 ± 8.266 | 0.196 |
| OTHER % | 0 ± 0 | 0 ± 0 | N/A | 2.667 ± 1.155 | 5.500 ± 3.697 | 0.359 NP |
| REACTIVE % | 0 ± 0 | 0 ± 0 | N/A | 0.333 ± 0.577 | 0.000 ± 0.000 | 0.386 NP |
| RES EP % | 0 ± 0 | 0 ± 0 | N/A | 0.000 ± 0.000 | 0.000 ± 0.000 | N/A |
| RBC | 0 ± 0 | 0 ± 0 | N/A | 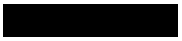 | 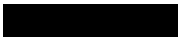 | 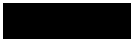 |
| WBC | 0 ± 0 | 0 ± 0 | N/A | 528.7 ± 365.5 | 556.0 ± 110.8 | 0.890 |
| BrY/Y (mmol/mol) | 0.123 ± 0.027 | 0.107 ± 0.023 | 0.309 | 0.574 ± 0.617 | 0.128 ± 0.044 | 0.227 NP |
| ClY/Y (mmol/mol) | 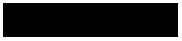 | 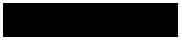 | 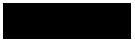 | 12.56 ± 17.19 | 0.328 ± 0.327 | 0.270 NP |
| DiY/Y (mmol/mol) | 8.117 ± 5.780 | 9.845 ± 7.479 | 0.690 | 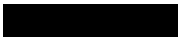 | 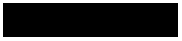 | 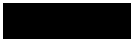 |
| NO2Y/Y (umol/mol) | 26.45 ± 14.53 | 23.36 ± 4.822 | 0.662 | 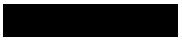 | 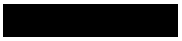 | 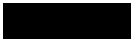 |
| mY/F (mmol/mol) | 0.472 ± 0.377 | 0.167 ± 0.096 | 0.158 | 0.468 ± 0.399 | 0.399 ± 0.234 | 0.949 NP |
| oY/P (mmol/mol) | 0.595 ± 0.300 | 0.797 ± 0.596 | 0.475 | 0.678 ± 0.469 | 0.314 ± 0.167 | 0.128 NP |
| | 16 hr/4 hr | | | 72 hr | | |
| | thioglycollate + zymosan | | | thioglycollate | | |
| | Cardax | NS | | Cardax | NS | |
| | Mean ± S.D. | Mean ± S.D. | P-Value | Mean ± S.D. | Mean ± S.D. | P-Value |
| AA (pmol) | 1208 ± 513 | 1258 ± 551 | 0.685 NP | 836.3 ± 441.3 | 735.9 ± 536.1 | 0.524 NP |
| LA (pmol) | 13158 ± 6225 | 14623 ± 2779 | 0.557 | 14242 ± 10380 | 12800 ± 8399 | 0.603 NP |
| PGF_{2α}(pmol) | 0.038 ± 0.014 | 0.058 ± 0.041 | 0.250 | 0.021 ± 0.021 | 0.025 ± 0.019 | 0.726 |
| PGF_{2α}(ratio) | 0.038 ± 0.026 | 0.067 ± 0.070 | 0.685 NP | 0.030 ± 0.026 | 0.040 ± 0.032 | 0.542 |
| 11-HETE (pmol) | 0.654 ± 0.400 | 0.614 ± 0.401 | 0.862 NP | 0.112 ± 0.069 | 0.101 ± 0.055 | 0.741 |
| 11-HETE (ratio) | 0.669 ± 0.633 | 0.588 ± 0.441 | 0.954 NP | 0.151 ± 0.091 | 0.193 ± 0.140 | 0.512 |
| 12-HETE (pmol) | 2.483 ± 2.347 | 7.298 ± 7.184 | 0.385 NP | 3.896 ± 2.022 | 2.167 ± 1.922 | 0.113 |
| 12-HETE (ratio) | 2.209 ± 1.716 | 5.817 ± 4.435 | 0.065 | 5.055 ± 3.001 | 4.765 ± 4.674 | 0.891 |
| 13-HODE (pmol) | 5.689 ± 4.149 | 11.90 ± 10.43 | 0.165 | 5.552 ± 3.243 | 4.319 ± 2.116 | 0.393 |
| 13-HODE (ratio) | 0.456 ± 0.249 | 0.939 ± 0.906 | 0.772 NP | 0.504 ± 0.393 | 0.496 ± 0.320 | 0.968 |
| 15-HETE (pmol) | 2.739 ± 1.089 | 4.112 ± 3.029 | 0.278 | 1.018 ± 0.605 | 0.537 ± 0.411 | 0.091 |
| 15-HETE (ratio) | 2.605 ± 1.417 | 3.691 ± 2.155 | 0.277 | 1.179 ± 0.457 | 1.161 ± 0.874 | 0.962 |
| 5,6-EET (pmol) | 0.092 ± 0.037 | 0.059 ± 0.024 | 0.056 | 0.055 ± 0.048 | 0.041 ± 0.050 | 0.450 NP |
| 5,6-EET (ratio) | 0.088 ± 0.057 | 0.055 ± 0.026 | 0.160 | 0.060 ± 0.028 | 0.068 ± 0.074 | 0.791 |
| 5-HETE (pmol) | 1.082 ± 0.502 | 1.128 ± 0.820 | 0.900 | 0.129 ± 0.103 | 0.080 ± 0.050 | 0.325 NP |
| 5-HETE (ratio) | 0.997 ± 0.601 | 1.231 ± 1.461 | 1.000 NP | 0.170 ± 0.134 | 0.153 ± 0.123 | 0.793 |
| 8-HETE (pmol) | 0.270 ± 0.116 | 0.425 ± 0.338 | 0.452 NP | 0.215 ± 0.101 | 0.134 ± 0.108 | 0.148 NP |
| 8-HETE (ratio) | 0.263 ± 0.196 | 0.383 ± 0.232 | 0.452 NP | 0.272 ± 0.122 | 0.215 ± 0.129 | 0.391 |
| 9-HETE (pmol) | 0.559 ± 0.198 | 0.735 ± 0.445 | 0.354 | 0.274 ± 0.147 | 0.164 ± 0.105 | 0.117 |
| 9-HETE (ratio) | 0.529 ± 0.313 | 0.665 ± 0.338 | 0.272 NP | 0.348 ± 0.191 | 0.336 ± 0.270 | 0.862 NP |
| 9-HODE (pmol) | 2.767 ± 1.006 | 3.350 ± 1.072 | 0.299 | 1.831 ± 1.130 | 2.481 ± 1.417 | 0.349 |
| 9-HODE (ratio) | 0.245 ± 0.142 | 0.251 ± 0.137 | 0.862 NP | 0.180 ± 0.163 | 0.285 ± 0.188 | 0.275 |
| 8-iso-F_{2α}(pmol) | 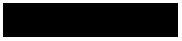 | 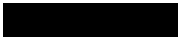 | 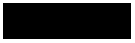 | 0.010 ± 0.014 | 0.005 ± 0.014 | 0.334 NP |
| 8-iso-F_{2α}(ratio) | 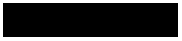 | 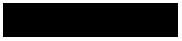 | 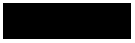 | 0.016 ± 0.021 | 0.013 ± 0.038 | 0.334 NP |
| 5-oxo-ETE (pmol) | 0.085 ± 0.026 | 0.091 ± 0.058 | 0.815 | 0.026 ± 0.051 | 0.016 ± 0.041 | 0.824 NP |
| 5-oxo-ETE (ratio) | 0.080 ± 0.040 | 0.092 ± 0.098 | 0.954 NP | 0.021 ± 0.035 | 0.024 ± 0.044 | 0.941 NP |
| ALV MAC % | 0 ± 0 | 0 ± 0 | N/A | 15.67 ± 5.033 | 12.67 ± 10.07 | 0.668 |
| EOSIN % | 0 ± 0 | 0 ± 0 | N/A | 1.667 ± 2.082 | 3.750 ± 2.754 | 0.326 |
| LYMPHS % | 0 ± 0 | 0 ± 0 | N/A | 53.00 ± 7.211 | 50.50 ± 18.70 | 0.838 |
| MONOS % | 0 ± 0 | 0 ± 0 | N/A | 27.00 ± 9.539 | 32.25 ± 14.17 | 0.607 |
| NEUTRO % | 0 ± 0 | 0 ± 0 | N/A | 0.000 ± 0.000 | 2.000 ± 2.708 | 0.267 |
| OTHER % | 0 ± 0 | 0 ± 0 | N/A | 2.333 ± 0.577 | 0.750 ± 0.957 | 0.095 NP |
| REACTIVE % | 0 ± 0 | 0 ± 0 | N/A | 0.333 ± 0.577 | 1.500 ± 1.291 | 0.266 NP |
| RES EP % | 0 ± 0 | 0 ± 0 | N/A | 0.000 ± 0.000 | 0.500 ± 1.000 | 0.564 NP |
| RBC | 0 ± 0 | 0 ± 0 | N/A | 90.33 ± 4.136 | 32.16 ± 3.130 | 0.152 |
| WBC | 0 ± 0 | 0 ± 0 | N/A | 435.3 ± 134.4 | 316.5 ± 83.5 | 0.377 NP |
| BrY/Y (mmol/mol) | 0.162 ± 0.047 | 0.148 ± 0.041 | 0.585 | 1.291 ± 2.826 | 0.177 ± 0.082 | 0.452 NP |
| ClY/Y (mmol/mol) | 0.156 ± 0.116 | 0.078 ± 0.041 | 0.125 | 5.932 ± 15.213 | 0.208 ± 0.104 | 1.000 NP |
| DiY/Y (mmol/mol) | 1.057 ± 0.720 | 1.096 ± 1.432 | 0.330 NP | 5.642 ± 2.786 | 7.186 ± 6.823 | 0.954 NP |
| NO2Y/Y (umol/mol) | 5.476 ± 4.227 | 6.713 ± 5.976 | 0.709 | 54.68 ± 44.16 | 38.78 ± 21.22 | 0.380 |
| mY/F (mmol/mol) | 0.258 ± 0.161 | 0.397 ± 0.264 | 0.637 NP | 0.217 ± 0.150 | 0.275 ± 0.153 | 0.272 NP |
| oY/P (mmol/mol) | 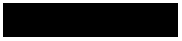 | 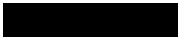 | 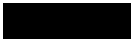 | 0.257 ± 0.146 | 0.281 ± 0.131 | 0.742 |

| | 72 hr/4 hr | | |
|---|---|---|---|
| | thioglycollate + zymosan | | |
| | Cardax | NS | |
| | Mean ± S.D. | Mean ± S.D. | P-Value |
| AA (pmol) | 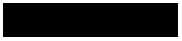 | 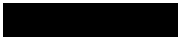 | 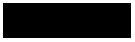 |
| LA (pmol) | 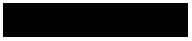 | 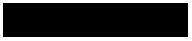 | 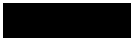 |
| PGF_{2α}(pmol) | 0.040 ± 0.033 | 0.047 ± 0.023 | 0.629 |
| PGF_{2α}(ratio) | 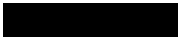 | 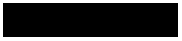 | 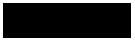 |
| 11-HETE (pmol) | 0.837 ± 0.553 | 0.651 ± 0.237 | 0.862 NP |
| 11-HETE (ratio) | 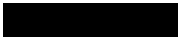 | 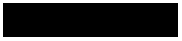 | 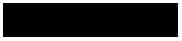 |
| 12-HETE (pmol) | 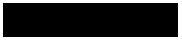 | 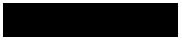 | 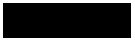 |
| 12-HETE (ratio) | 1.452 ± 0.773 | 1.582 ± 1.336 | 0.603 NP |
| 13-HODE (pmol) | 7.181 ± 3.663 | 5.543 ± 1.603 | 0.271 |
| 13-HODE (ratio) | 0.229 ± 0.078 | 0.297 ± 0.050 | 0.118 NP |
| 15-HETE (pmol) | 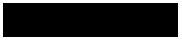 | 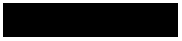 | 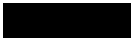 |
| 15-HETE (ratio) | 1.453 ± 0.420 | 1.788 ± 0.455 | 0.164 |
| 5,6-EET (pmol) | 0.384 ± 0.849 | 0.090 ± 0.048 | 0.685 NP |
| 5,6-EET (ratio) | 0.061 ± 0.094 | 0.059 ± 0.020 | 0.056 NP |
| 5-HETE (pmol) | 1.236 ± 0.754 | 1.040 ± 0.405 | 0.385 NP |
| 5-HETE (ratio) | 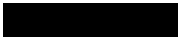 | 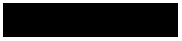 | 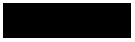 |
| 8-HETE (pmol) | 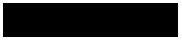 | 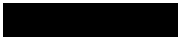 | 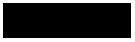 |
| 8-HETE (ratio) | 0.138 ± 0.041 | 0.159 ± 0.035 | 0.183 NP |
| 9-HETE (pmol) | 0.917 ± 0.781 | 0.505 ± 0.136 | 0.116 NP |
| 9-HETE (ratio) | 0.253 ± 0.071 | 0.354 ± 0.091 | 0.056 NP |
| 9-HODE (pmol) | 4.747 ± 4.406 | 3.750 ± 1.284 | 0.772 NP |
| 9-HODE (ratio) | 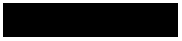 | 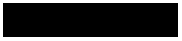 | 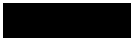 |
| 8-iso-F_{2α}(pmol) | 0.057 ± 0.062 | 0.072 ± 0.141 | 0.502 NP |
| 8-iso-F_{2α}(ratio) | 0.017 ± 0.023 | 0.070 ± 0.147 | 0.760 NP |
| 5-oxo-ETE (pmol) | 0.148 ± 0.181 | 0.495 ± 0.777 | 0.073 NP |
| 5-oxo-ETE (ratio) | 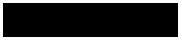 | 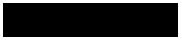 | 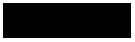 |
| ALV MAC % | 0 ± 0 | 0 ± 0 | N/A |
| EOSIN % | 0 ± 0 | 0 ± 0 | N/A |
| LYMPHS % | 0 ± 0 | 0 ± 0 | N/A |
| MONOS % | 0 ± 0 | 0 ± 0 | N/A |
| NEUTRO % | 0 ± 0 | 0 ± 0 | N/A |
| OTHER % | 0 ± 0 | 0 ± 0 | N/A |
| REACTIVE % | 0 ± 0 | 0 ± 0 | N/A |
| RES EP % | 0 ± 0 | 0 ± 0 | N/A |
| RBC | 0 ± 0 | 0 ± 0 | N/A |
| WBC | 0 ± 0 | 0 ± 0 | N/A |
| BrY/Y (mmol/mol) | 0.211 ± 0.114 | 0.198 ± 0.081 | 0.862 NP |
| ClY/Y (mmol/mol) | 0.085 ± 0.032 | 0.087 ± 0.043 | 0.904 |
| DiY/Y (mmol/mol) | 0.543 ± 0.119 | 0.417 ± 0.116 | 0.059 |
| NO2Y/Y (umol/mol) | 8.618 ± 9.430 | 12.70 ± 14.24 | 0.385 NP |
| mY/F (mmol/mol) | 0.243 ± 0.286 | 0.343 ± 0.401 | 0.118 NP |
| oY/P (mmol/mol) | 0.249 ± 0.345 | 0.162 ± 0.100 | 0.385 NP |

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

### REFERENCES

The following references are specifically incorporated herein by reference:

### U.S. patent applications

U.S. patent application Ser. No.: 10/793,670; Filed: Mar. 4, 2004; Inventor(s): Lockwood et al.; Title: “CAROTENOID ANALOGS OR DERIVATIVES FOR THE INHIBITION AND AMELIORATION OF DISEASE”

### U.S. patent Documents

U.S. Pat. No. 5,871,766 February 1999 Hennekens

### OTHER REFERENCES

Axford-Gatley, R. A, and Wilson, J. G. (1991). Reduction of experimental myocardial infarct size by oral administration of alpha-tocopherol. Cardiovasc Res 25: 89-92.
Baxter, G. F., and Yellon, D. M. (1993). Attenuation of reperfusion-induced ventricular fibrillation in the rat isolated hypertrophied heart by preischemic diltiazem treatment. Cardiovasc Drugs Ther 7: 225-231.
Blaustein, A. S., Schine, L., Brooks, W. W., Franburg, B. L, and Bing, O. H. (1986). Influence of exogenously generated oxidant species on myocardial function. Am J Physiol 250: H595-H599.
Bolli, R., Patel, B. S., Zhu, W. X., et al. (1987). The iron chelator desferioxamine attenuates postischemic ventricular dysfunction. Am J Physiol 253: H1372-H1380.
Bolli, R., Zhu, W. X., and Hartley, C. J. (1987). Attenuation of dysfunction in the postischemic “stunned” myocardium by dimethylurea. Circulation 76: 458-468.
Bolli, R., Jeroudi, M. O., Patel, B. S., et al. (1989). Direct evidence that oxygen-derived free radicals contribute to post-ischemic myocardial dysfunction in the intact dog. Proc Natl Acad Sci USA 86: 4695-4699.
Chopra, M., McMurray, J., Stewart, J., Dargie, H. J., and Smith, W. E. (1990). Free radical scavenging: potentially beneficial action of thiol-containing angiotensin-converting enzyme inhibitors. Biochem Soc Trans 18: 1184-1185.
Conorev, E. A., Sharov, V. G., and Saks, V. A. (1991). Improvement of contractile recovery of isolated rat heart after cardioplegic ischemic arrest with endogenous phosphocreatine: involvement of antiperoxidative effect? Cardiovasc Res 25: 164-171.
Duilio, C., Ambrosio, G., Kuppusamy, P., Dipaula, A., Becker, L. C., and Zweier, J. L. (2001). Neutrophils are primary source of O₂ radicals during reperfusion after prolonged myocardial ischemia. Am J Physiol Heart Circ Physiol 280: H2649-H2657.
Goto, S., Kogure, K., Abe, K., Kimata, Y., Kitahama, K., Yamashita, E., and Terada, H. (2001). Efficient radical trapping at the surface and inside the phospholipids membrane is responsible for highly potent antiperoxidative activity of the carotenoid astaxanthin. Biochimica et Biophysica Acta 1512: 251-258.
Hearse, D. J., Manning, A. S., Downey, J. M., and Yellon, D. M. (1986). Xanthine oxidase: a critical mediator of myocardial injury during ischemia and reperfusion. Acta Physiol Scand 548: 65-74.
Horwitz, L. D., Kong, Y., and Robertson, A. D. (1999). Timing of treatment for myocardial reperfusion injury. J Cardiovasc Pharmacol 33 (1): 19-29.
Johansson, M. H., Deinum, J., Marklund, S. L., and Sjodquist, P. O. (1990). Recombinant human extracellular superoxide dismutase reduces concentration of oxygen free radicals in the reperftised rat heart. Cardiovasc Res 24: 500-503.
Kimura, Y., Engelman, R. M., Rousou, J., Flack, J., Iyengar, J., and Das, D. K. (1992). Moderation of myocardial ischemia reperfusion injury by calcium channel and calmodulin receptor inhibition. Heart Vessels 7: 189-195.
Levy, Y., Bartha, P., Ben-Amotz, A., Brook, J. G., Dankner, G., Lin, S., and Hammerman, H. (1998). Plasma antioxidants and lipid peroxidation in acute myocardial infarction and thrombolysis. J Am Coll Nutr 17 (4): 337-341.
Mahaffey, K. W., Puma, J. A., Barbagelata, N. A., DiCarli, M. F., Leesar, M. A., Browne, K. F., Eisenberg, P. R., Bolli, R., Casas, C., Molina-Viamonte, V., Orlandi, C., Blevins, R., Gibbons, R. J., Califf, R. M., Granger, C. B. (1999). Adenosine as an adjunct to thrombolytic therapy for acute myocardial infarction. J Am Coll Cardiol 34(6): 1711-1720.
McMurray, J., and Chopra, M. (1991). Influence of ACE inhibitors on free radicals and reperfusion injury: pharmacological curiosity or therapeutic hope? Br J Pharmacol 31: 373-379.
Petty, M. A., Dow, J., Grisar, J. M., and De-Jong, W. Effect of a cardioselective alpha-tocopherol analogue on reperfusion injury in rats induced by myocardial ischemia. Eur J Pharmacol 192: 383-388.
Sajkowska, A., Wykretowicz, A., Szczepanik, A., Kempa, M., Minczykowski, A., and Wysocki, H. (1999). Fibrinolytic therapy and n-acetylcysteine in the treatment of patients with acute myocardial infarction: its influence on authentic plasma hydroperoxide levels and polymorphonuclear neutrophil oxygen metabolism. Cardiology 91: 60-65.
Schaer, G. L., Spaccavento, L. J., Browne, K. F., Krueger, K. A., Krichbau, D., Phelan, J. M., Fletcher, W. O., Grines, C. L., Edwards, S., Jolly, M. K., and Gibbons, R. J. (1996). Beneficial effects of RheothRx injection in patients receiving thrombolytic therapy for acute myocardial infarction. Results of a randomized, double-blind, placebo-controlled trial. Circulation 94(3): 298-307.
Sethi, R., Takeda, N., Nagano, M., and Dhalla, N. S. (2000). Beneficial effects of vitamin E treatment in acute myocardial infarction. J Cardiovasc Pharmacol Ther 5: 51-58.
Shuter, S. L., Davies, M. J., Garlick, P. B, Hearse, D. J., and Slater, T. F. (1990). Studies on the effects of of antioxidants and inhibitors of radical generation on free radical production in the reperfused rat heart using electron spin resonance spectroscopy. Free Radic Res Commun 9: 223-232.
Simpson, P. J., and Lucchesi, B. R. (1987). Free radicals and myocardial ischemia and reperfusion injury. J Lab Clin Med 110: 13-30.
Singh, R. B., Niaz, M. A., Sharma, J. P., Kumar, R., Bishnoi, I., and Begom, R. (1994). Plasma levels of antioxidant vitamins and oxidative stress in patients with acute myocardial infarction. Acta Cardiologica Vol. XLIX 5: 441-452.
Such, L., Morcillo, E., Chorro, F. J., et al. Beneficial effects of N-acetylcysteine on acute myocardial infarction in open chest dogs. Arch Pharmacol Toxicol 12: 37-40.
Rogers, M., Berestecky, J. M., Hossain, M. Z., Guo, H., Kadle, R., Nicholson, B. J., and Bertram, J. S. (1990). Retinoid-enhanced gap junctional communication is achieved by increased levels of connexin 43 mRNA and protein. Molecular Carcinogenesis 3: 335-343.
Bertram, J. S. (1999). Carotenoids and gene regulation. Nutrition Reviews 57(6): 182-191.
Gutstein, D. E., Morley, G. E., Tamaddon, H., Vaidya, D., Schneider, M. D., Chen, J., Chien, K. R., Stuhlmann, H., Fishman, G. I. (2001). Conduction slowing and sudden arrhythmic death in mice with cardiac-restricted inactivation of connexin 43. Circulation Research: 333-339.
Dulio, C., Ambrosio, G., Kuppusamy, P., Dipaula, A., Becker, L. C., Zweier, J. L. (2001). Neutrophils are primary source of O₂ radicals during reperfusion after prolonged myocardial ischemia. Am J Physiol Heart Circ Physiol 280: H2649-H2657.
Liao, M.-L., Wang, S. Y., Chung, C., Liang, Y.-T., Sejb, P. A., (1988). Synthesis of L-Ascorbate 6-Phosphate. Carbohydrate Research 176: 73-77.
Bock, K., Lundt, I., Pedersen, C., (1979). Preparation of Some Bromodeoxyaldonic Acids. Carbohydrate Research 68: 313-319.
Stuber, H. A., Tolbert, B. M., (1978). A New Synthesis of L-threo-Hex-2-Enaro-1,4-Lactone (“Saccharoascorbic” Acid): A Method for the Protection of the Enediol of Ascorbic Acid. Carbohydrate Research 60: 251-258.
G. Britton, Structure and properties of carotenoids in relation to function, FASEB J. 9 (1995) 1551-1558.
A. M. Papas, Antioxidant status: diet, health and disease; Part I: Factors affecting antioxidant status and its role, Mature Medicine (1999) 315-319.
W. Miki, Biological functions and activities of animal carotenoids, Pure Appl. Chemistry 63 (1991) 141-146.
S. F. Lockwood, S. O'Malley, G. L. Mosher, Improved aqueous solubility of crystalline astaxanthin (3,3′-dihydroxy-beta, beta-carotene-4,4′-dione) by Captisol(R) (sulfobutyl ether beta-cyclodextrin), J. Pharm. Sci. 92 (2003) 922-926.
M. Buchwaldt, W. P. Jencks, Optical properties of astaxanthin solutions and aggregates, Biochemistry 7 (1968) 834-843.
V. Salares, N. Young, P. Carey, H. Bernstein, Excited state (exciton) interactions in polyene aggregates, J. Raman Spectr. 6 (1977) 282-288.
R. K. Müller, K. Bernhard, H. Mayer, A. Ruttimann, M. Vecchi, Contribution to the analytical separation and the synthesis of 3-hydroxy-4-oxocarotenoids, Helv. Chim. Acta 63 (1980) 1654-1664.
T. Peters, All About Albumin, Academic Press, San Diego (Calif.), 1996.
H. Watanabe, S. Tanase, K. Nakajou, T. Maruyama, U. Kragh-Hansen, M. Otagiri, Role of Arg-410 and Tyr-411 in human serum albumin for ligand binding and esterase-like activity, Biochem. J. 349 (2000) 813-819.
U. Kragh-Hansen, V. Chuang, M. Otagiri, Practical aspects of the ligand-binding and enzymatic properties of human serum albumin, Biol. Pharm. Bull. 25 (2002) 695-704.
K. Bernhard, G. Englert, H. Mayer, R. K. Muller, A. Ruttimann, M. Vecchi, E. Widmer E, R. Zell, Synthesis of optically-active natural carotenoids and structurally related-compounds. 9. Synthesis of (3R)-hydroxyechinenone, (3R,3′R)-adonixanthin and (3R,3′S)-adonixanthin, (3R)-adonirubin, their optical antipodes and related-compounds, Helv. Chim. Acta 64 (1981) 2469-2484.
V. Sturzenegger, R. Buchecker, G. Wagniere, Classification of the CD spectra of carotenoids, Helv. Chim. Acta 63 (1980) 1074-1092.
A. G. Andrewes, G. Borch, S. Liaaen-Jensen, G. Snatzke, Animal carotenoids. 9. On the absolute configuration of astaxanthin and actinioerythrin, Acta Chem. Scand. B 28 (1974) 730-736.
B. F. Lutnaes, O. R. Gautun, S. Liaaen-Jensen, Is (9Z)-“meso”-zeaxanthin optically active? Chirality 13 (2001) 224-229.
Z. Bikádi, F. Zsila, J. Deli, G. Mády, M. Simonyi, The supramolecular structure of self-assembly formed by capsanthin derivatives, Enantiomer 7 (2002) 67-76.
K. Noack, A. J. Thomson, Conformation and optical-activity of all-trans, mono-cis, and di-cis carotenoids—temperature-dependent circular-dichroism, Helv. Chim. Acta 62 (1979) 1902-1921.
K. Noack, A. J. Thomson, Temperature and concentration dependent circular-dichroism of mono-cis and di-cis isomers of (3R,3′S)-astaxanthin diacetate, Helv. Chim. Acta 64 (1981) 2383-2392.
N. Harada, K. Nakanishi, Circular Dichroic Spectroscopy—Exciton Coupling in Organic Stereochemistry, University Science Books, Mill Valley (Calif.), 1983.
N. Harada, Y. Takuma, H. Uda, Circular dichroic power due to chiral exciton coupling between two polyacene chromophores, J. Am. Chem. Soc. 100 (1978) 4029-4036.
J-K. Choi, J. Ho, S. Curry, D. Qin, R. Bittman, J. Hamilton, Interactions of very long-chain saturated fatty acids with serum albumin, J. Lipid Res. 43 (2002) 1000-1010.
S. Curry, P. Brick, N. P. Franks, Fatty acid binding to human serum albumin: new insights from crystallographic studies, Biochim. Biophys. Acta 1441 (1999) 131-140.
Petitpas, T. Grüne, A. Bhattacharya, S. Curry, Crystal structures of human serum albumin complexed with monounsaturated and polyunsaturated fatty acids, J. Mol. Biol. 314 (2001) 955-960.
A. A. Bhattacharya, T. Grüne, S. Curry, Crystallographic analysis reveals common modes of binding of medium and long-chain fatty acids to human serum albumin, J. Mol. Biol. 303 (2000) 721-732.
Cross, C. E., B. Halliwell, E. T. Borish, W. A. Pryor, B. N. Ames, R. L. Saul, J. M. McCord, and D. Harman. (1987) Oxygen radicals and human disease. Ann. Intern. Med., 107:526-545.
Zhang, L.-X., Cooney, R. V., and Bertram, J. S. (1992) Carotenoids up-regulate connexin 43 gene expression independent of their pro-vitamin A or antioxidant properties. Cancer Res., 52, 5707-5712.
Aoi, W.; Naito, Y.; Sakuma, K.; Kuchide, M.; Tokuda, H.; Maoka, T.; Toyokuni, S.; Oka, S.; Yasuhara, M.; Yoshikawa, T. Astaxanthin limits exercise-induced skeletal and cardiac muscle damage in mice. Antioxid Redox Signal. 2003, 5, 139-144.
Bailey, J. M.; Bryant, R. W.; Whiting, J.; Salata, K. Characterization of 11-HETE and 15-HETE, together with prostacyclin, as major products of the cyclooxygenase pathway in cultured rat aorta smooth muscle cells. J Lipid Res. 1983, 24, 1419-1428.
Beckman, K. B.; Ames, B. N. The free radical theory of aging matures. Physiol Rev. 1998, 78, 547-581.
Beutner, S.; Bloedorn, B.; Frixel, S.; Blanco, I. H.; Hoffmann, T.; Martin, H.-D.; Mayer, B.; Noack, P.; Ruck, C.; Schmidt, M.; Schülke, I.; Sell, S.; Ernst, H.; Haremza, S.; Seybold, G. n.; Sies, H.; Stahl, W.; Walsh, R. Quantitative assessment of antioxidant properties of natural colorants and phytochemicals: Carotenoids, .Avonoids, phenols and indigoids. The role of β-carotene in antioxidant functions. J Sci Food Agric. 2001, 81, 559-568.
Bikádi, Z.; Zsila, F.; Deli, J.; Mady, G.; Simonyi, M. The supramolecular structure of self-assembly formed by capsanthin derivatives. Enantiomer. 2002, 7, 67-76.
Britton, G. Structure and properties of carotenoids in relation to function. FASEB J. 1995, 9, 1551-1558.
Burton, G. W.; Ingold, K. U. β-Carotene: An unusual type of lipid antioxidant. Science. 1984, 224, 569-573.
Cardounel, A. J.; Dumitrescu, C.; Zweier, J. L.; Lockwood, S. F. Direct superoxide anion scavenging by a disodium disuccinate astaxanthin derivative: Relative efficacy of individual stereoisomers versus the statistical mixture of stereoisomers by electron paramagnetic resonance imaging. Biochem Biophys Res Commun. 2003, 307, 704-712.
Cipollone, F.; Mezzetti, A.; Fazia, M. L.; Cuccurullo, C.; Iezzi, A.; Ucchino, S.; Spigonardo, F.; Bucci, M.; Cuccurullo, F.; Prescott, S. M.; Stafforini, D. M. Association between 5-lipoxygenase expression and plaque instability in humans. Arterioscler Thromb Vasc Biol. 2005, 25, 1665-1670.
Devaraj, S.; Jialal, I. α-Tocopherol decreases tumor necrosis factor-α mRNA and protein from activated human monocytes by inhibition of 5-lipoxygenase. Free Radic Biol Med. 2005, 38, 1212-1220.
Drazen, J. M. Asthma therapy with agents preventing leukotriene synthesis or action. Proc Assoc Am Physicians. 1999, 111, 547-559.
Foss, B. J.; Sliwka, H. R.; Partali, V.; Naess, S. N.; Elgsaeter, A.; Melo, T. B.; Naqvi, K. R.; O'Malley, S.; Lockwood, S. F. Hydrophilic carotenoids: Surface properties and aqueous aggregation of a rigid, long-chain, highly unsaturated dianionic bolaamphiphile with a carotenoid spacer. Chem Phys Lipids. 2005, 135, 157-167.
Frank, H. A.; Bautista, J. A.; Josue, J.; Pendon, Z.; Hiller, R. G.; Sharples, F. P.; Gosztola, D.; Wasielewski, M. R. Effect of the solvent environment on the spectroscopic properties and dynamics of the lowest excited states of carotenoids. J Phys Chem B. 2000, 104, 4569 -4577.
Frey, D. A.; Kataisto, E. W.; Ekmanis, J. L.; O'Malley, S.; Lockwood, S. F. The efficient synthesis of disodium disuccinate astaxanthin (Cardax). Org Process Res Dev. 2004, 8, 796-801.
Gross, G. J.; Lockwood, S. F. Cardioprotection and myocardial salvage by a disodium disuccinate astaxanthin derivative (Cardax™). Life Sci. 2004, 75, 215-224.
Gross, G. J.; Hazen, S. L.; Lockwood, S. F. Seven day oral supplementation with Cardax™ (disodium disuccinate astaxanthin) provides significant cardioprotection and reduces oxidative stress in rats. 2005, in press.
Gross, G. J.; Lockwood, S. F. Acute and chronic administration of disodium disuccinate astaxanthin (Cardax™) produces marked cardioprotection in dog hearts. Mol Cell Biochem. 2005, 272, 221-227.
Hakonarson, H.; Thorvaldsson, S.; Helgadottir, A.; Gudbjartsson, D.; Zink, F.; Andresdottir, M.; Manolescu, A.; Arnar, D. O.; Andersen, K.; Sigurdsson, A.; Thorgeirsson, G.; Jonsson, A.; Agnarsson, U.; Bjornsdottir, H.; Gottskalksson, G.; Einarsson, A.; Gudmundsdottir, H.; Adalsteinsdottir, A. E.; Gudmundsson, K.; Kristjansson, K.; Hardarson, T.; Kristinsson, A.; Topol, E. J.; Gulcher, J.; Kong, A.; Gurney, M.; Stefansson, K. Effects of a 5-lipoxygenase-activating protein inhibitor on biomarkers associated with risk of myocardial infarction: A randomized trial. JAMA. 2005, 293, 2245-2256.
Harada, N.; Nakanishi, K. The exciton chirality method and its application to configurational and conformational studies of natural products. Acc Chem Res. 1972, 5, 257-263.
Helmersson, J.; Vessby, B.; Larsson, A.; Basu, S. Cyclooxygenase-mediated prostaglandin F_{2α} is decreased in an elderly population treated with low-dose aspirin. Prostaglandins Leukot Essent Fatty Acids. 2005, 72, 227-233.
Jiang, Q.; Ames, B. N. γ-Tocopherol, but not α-tocopherol, decreases proinflammatory eicosanoids and inflammation damage in rats. FASEB J. 2003, 17, 816-822.
Jouni, Z. E.; Wells, M. A. Purification and partial characterization of a lutein-binding protein from the midgut of the silkworm Bombyx mori. J Biol Chem. 1996, 271, 14722-14726.
Kühn, H.; Belkner, J.; Wiesner, R.; Schewe, T.; Lankin, V. Z.; Tikhaze, A. K. Structure elucidation of oxygenated lipids in human atherosclerotic lesions. Eicosanoids. 1992, 5, 17-22.
Lauver, D. A.; Lockwood, S. F.; Lucchesi, B. R. Disodium disuccinate astaxanthin (Cardax) attenuates complement activation and reduces myocardial injury following ischemia/reperfusion. J Pharmacol Exp Ther. 2005, 314, 686-692.
Lee, S. J.; Bai, S. K.; Lee, K. S.; Namkoong, S.; Na, H. J.; Ha, K. S.; Han, J. A.; Yim, S. V.; Chang, K.; Kwon, Y. G.; Lee, S. K.; Kim, Y. M. Astaxanthin inhibits nitric oxide production and inflammatory gene expression by suppressing IκB kinase-dependent NF-κB activation. Mol Cells. 2003, 16, 97-105.
Libby, P.; Theroux, P. Pathophysiology of coronary artery disease. Circulation. 2005, 111, 3481-3488.
Lightner, D. A.; Gurst, J. E. Organic conformational analysis and stereochemistry from circular. In Dichroism spectroscopy. John Wiley: New York, 2000, p. Chapter 14.
Lötzer, K.; Funk, C. D.; Habenicht, A. J. The 5-lipoxygenase pathway in arterial wall biology and atherosclerosis. Biochim Biophys Acta. 2005, in press.
Mehrabian, M.; Allayee, H.; Wong, J.; Shi, W.; Wang, X. P.; Shaposhnik, Z.; Funk, C. D.; Lusis, A. J. Identification of 5-lipoxygenase as a major gene contributing to atherosclerosis susceptibility in mice. Circ Res. 2002, 91, 120-126.
Miki, W. Biological functions and activities of animal carotenoids. Pure Appl Chem. 1991, 63, 141-146.
Ohgami, K.; Shiratori, K.; Kotake, S.; Nishida, T.; Mizuki, N.; Yazawa, K.; Ohno, S. Effects of astaxanthin on lipopolysaccharide-induced inflammation in vitro and in vivo. Invest Ophthalmol Vis Sci. 2003, 44, 2694-2701.
Roberts, L. J., 2nd; Morrow, J. D. Products of the isoprostane pathway: Unique bioactive compounds and markers of lipid peroxidation. Cell Mol Life Sci. 2002, 59, 808-820.
Shimidzu, N.; Goto, M.; Miki, W. Carotenoids as singlet oxygen quenchers in marine organisms. Fisheries Science. 1996, 62, 134-137.
Shishehbor, M. H.; Brennan, M. L.; Aviles, R. J.; Fu, X.; Penn, M. S.; Sprecher, D. L.; Hazen, S. L. Statins promote potent systemic antioxidant effects through specific inflammatory pathways. Circulation. 2003, 108, 426-431.
Showalter, L. A.; Weinman, S. A.; Osterlie, M.; Lockwood, S. F. Plasma appearance and tissue accumulation of non-esterified, free astaxanthin in C57BL/6 mice after oral dosing of a disodium disuccinate diester of astaxanthin (Heptax™). Comp Biochem Physiol C Toxicol Pharmacol. 2004, 137, 227-236.
Singh, U.; Devaraj, S.; Jialal, I. Vitamin E, oxidative stress, and inflammation. Annu Rev Nutr. 2005, 25, 151-174.
Spanbroek, R.; Grabner, R.; Lotzer, K.; Hildner, M.; Urbach, A.; Ruhling, K.; Moos, M. P.; Kaiser, B.; Cohnert, T. U.; Wahlers, T.; Zieske, A.; Plenz, G.; Robenek, H.; Salbach, P.; Kuhn, H.; Radmark, O.; Samuelsson, B.; Habenicht, A. J. Expanding expression of the 5-lipoxygenase pathway within the arterial wall during human atherogenesis. Proc Natl Acad Sci USA. 2003, 100, 1238-1243.
Spector, A. A.; Gordon, J. A.; Moore, S. A. Hydroxyeicosatetraenoic acids (HETEs). Prog Lipid Res. 1988, 27, 271-323.
Steinberg, D.; Witztum, J. L. Is the oxidative modification hypothesis relevant to human atherosclerosis? Do the antioxidant trials conducted to date refute the hypothesis? Circulation. 2002, 105, 2107-2111.
Waddington, E.; Sienuarine, K.; Puddey, I.; Croft, K. Identification and quantitation of unique fatty acid oxidation products in human atherosclerotic plaque using high-performance liquid chromatography. Anal Biochem. 2001, 292, 234-244.
Zhang, R.; Brennan, M. L.; Shen, Z.; MacPherson, J. C.; Schmitt, D.; Molenda, C. E.; Hazen, S. L. Myeloperoxidase functions as a major enzymatic catalyst for initiation of lipid peroxidation at sites of inflammation. J Biol Chem. 2002, 277, 46116-46122.
Zigmantas, D.; Hiller, R. G.; Sharples, F. P.; Frank, H. A.; Sundstrom, V.; Polívka, T. Effect of a conjugated carbonyl group on the photophysical properties of carotenoids. Phys Chem Chem Phys. 2004, 6, 3009-3016.
Zimpfer, U.; Hofmann, C.; Dichmann, S.; Schopf, E.; Norgauer, J. Synthesis, biological effects and pathophysiological implications of the novel arachidonic acid metabolite 5-oxo-eicosatetraenoic acid (review). Int J Mol Med. 1998,2, 149-153.
Zsila, F.; Bikádi, Z.; Keresztes, Z.; Deli, J.; Simonyi, M. Investigation of the self-organization of lutein and lutein diacetate by electronic absorption, circular dichroism spectroscopy, and atomic force microscopy. J Phys Chem B. 2001, 105, 9413-9421.
Zsila, F.; Simonyi, M.; Lockwood, S. F. Interaction of the disodium disuccinate derivative of meso-astaxanthin with human serum albumin: From chiral complexation to self-assembly. Bioorg Med Chem Lett. 2003, 13, 4093-4100.
Zsila, F.; Fitos, I.; Bikadi, Z.; Simonyi, M.; Jackson, H. L.; Lockwood, S. F. In vitro plasma protein binding and aqueous aggregation behavior of astaxanthin dilysinate tetrahydrochloride. Bioorg Med Chem Lett. 2004, 14, 5357-5366.
Zsila, F.; Molnár, P.; Deli, J.; Lockwood, S. Circular dichroism and absorption spectroscopic data reveal binding of the natural cis-carotenoid bixin to human α₁-acid glycoprotein. Bioorganic Chemistry. 2005, in press.
Peters, N. S. (1995) Myocardial gap junction organization in ischemia and infarction. Microsc. Res. Tech., 31, 375-386.
Zhang, L.-X. and Bertram, J. S. (1994) Assays for Regulation of Gap Junctional Communication and Connexin expression by Carotenoids. In Packer, L. (ed.) Oxygen radicals in biological systems, Part C. In: Methods in Enzymology: Vol 234. Academic Press, Orlando, pp 235-44.
Hossain, M. Z., Zhang, L.-X., and Bertram, J. S. (1993) Retinoids and carotenoids upregulate gap junctional communication: correlation with enhanced growth control and cancer prevention. In Hall, J. E., Zampighi, G. A., and Davies, R. M. (eds.) Progess in Cell Research Vol. 3: Gap Junctions. Elsiever, Amsterdam, pp 301-9.
Perkins, G., Goodenough, D., and Sosinsky, G. (1997) Three-dimensional structure of the gap junction connexon. Biophysical Journal, 72, 533-544.
Saez, J. C., Martinez, A. D., Branes, M. C., and Gonzalez, H. E. (1998) Regulation of gap junctions by protein phosphorylation. Braz. J. Med. Biol. Res., 31, 593-600.
A. S. Moore, and A. M. Papas, Biochemistry and health significance of Vitamin E, J. Adv. Med. 9 (1996) 11-29.
P. Di Mascio, T. P. Devasagayam, S. Kaiser, and H. Sies, Carotenoids, tocopherols and thiols as biological singlet molecular oxygen quenchers, Biochem. Soc. Trans. 18 (1990) 1054-1056.
P. Di Mascio, M. E. Murphy, and H. Sies, Antioxidant defense systems: the role of carotenoids, tocopherols, and thiols, Am. J. Clin. Nutr. 53 (1991) 194S-200S.
J. H. Tinkler, F. Bo+EEHM, W. Schalch, and T. G. Truscott, Dietary carotenoids protect human cells from damage, J. Photochem. Photobiol. B. 26 (1994) 283-285. )}
H. Jyonouchi, S. Sun, K. Iijima, and M. D. Gross, Antitumor activity of astaxanthin and its mode of action, Nutr. Cancer 36 (2000) 59-65.
A. Kistler, H. Liechti, L. Pichard, E. Wolz, G. Oesterhelt, A. Hayes, and P. Maurel, Metabolism and CYP-inducer properties of astaxanthin in man and primary human hepatocytes, Arch. Toxicol. 75 (2002) 665-675.
A. Mortensen, L. H. Skibsted, J. Sampson, C. Rice-Evans, and S. A. Everett, Comparative mechanisms and rates of free radical scavenging by carotenoid antioxidants, FEBS Lett. 418 (1997) 91-97.
J. Terao, Antioxidant activity of beta-carotene-related carotenoids in solution., Lipids 24 (1989) 659-661.
P. Palozza, and N. I. Krinsky, Astaxanthin and canthaxanthin are potent antioxidants in a membrane model, Arch. Biochem. Biophys. 297 (1992) 291-295.
B. P. Lim, A. Nagao, J. Terao, K. Tanaka, T. Suzuki, and K. Takama, Antioxidant activity of xanthophylls on peroxyl radical-mediated phospholipid peroxidation, Biochim. Biophys. Acta 1126 (1992) 178-184.
B. N. Ames, M. K. Shigenaga, and T. M. Hagen, Oxidants, antioxidants, and the degenerative diseases of aging, Proc. Natl. Acad. Sci. U.S.A. 90 (1993) 7915-7922.
S. Mayne, Beta-carotene, carotenoids, and disease prevention in humans., FASEB J. 10 (1996) 690-701.
V. R. Salares, N. M. Young, H. J. Bernstein, and P. R. Carey, Resonance Raman spectra of lobster shell carotenoproteins and a model astaxanthin aggregate. A possible photobiological function for the yellow protein, Biochemistry 16 (1977) 4751-4756.
F. Zsila, J. Deli, Z. Bikadi, and M. Simonyi, Supramolecular assemblies of carotenoids, Chirality 13 (2001) 739-744.
Z. Bikadi, F. Zsila, J. Deli, G. Mady, and M. Simonyi, The supramolecular structure of self-assembly formed by capsanthin derivatives, Enantiomer 7 (2002) 67-76.
C. V. Serrano, Jr., E. A. Mikhail, P. Wang, B. Noble, P. Kuppusamy, and J. L. Zweier, Superoxide and hydrogen peroxide induce CD18-mediated adhesion in the postischemic heart, Biochim. Biophys. Acta 1316 (1996) 191-202.
E. W. Gabrielson, P. Kuppusamy, A. C. Povey, J. L. Zweier, and C. C. Harris, Measurement of neutrophil activation and epidermal cell toxicity by palytoxin and 12-O-tetradecanoylphorbol-13-acetate, Carcinogenesis 13 (1992) 1671-1674.
C. Lee, K. Miura, X. Liu, and J. L. Zweier, Biphasic regulation of leukocyte superoxide generation by nitric oxide and peroxynitrite, J. Biol. Chem. 275 (2000) 38965-38972.
M. Kurashige, E. Okimasu, M. Inoue, and K. Utsumi, Inhibition of oxidative injury of biological membranes by astaxanthin., Physiol. Chem. Phys. Med. NMR 22 (1990) 27-38.
E. Oliveros, A. M. Braun, T. Aminian-Saghafi, and H. R. Sliwka, Quenching of singlet oxygen by carotenoid derivatives: kinetic analysis by near-infrared luminescence, New J. Chem. 18 (1994) 535-539.
F. Zsila, J. Deli, and M. Simonyi, Color and chirality: carotenoid self-assemblies in flower petals, Planta 213 (2001) 937-942.
Osterlie M, Bjerkeng B, Liaaen-Jensen S. 2000. Plasma appearance and distribution of astaxanthin E/Z and R/S isomers in plasma lipoproteins of men after single dose administration of astaxanthin. J. Nutr. Biochem. 10: 482-490.
Jewell C, O'Brien N M. 1999. Effect of dietary supplementation with carotenoids on xenobiotic metabolizing enzymes in the liver, lung, kidney and small intestine of the rat. Br. J. Nutr. 81(3): 235-42.
Kurihara H, Koda H, Asami S, Kiso Y, Tanaka T. 2002. Contribution of the antioxidative property of astaxanthin to its protective effect on the promotion of cancer metastasis in mice treated with restraint stress. Life Sci. (21): 2509-20.
Kang J O, Kim S J, Kim H. 2001. Effect of astaxanthin on the hepatotoxicity, lipid peroxidation and antioxidative enzymes in the liver of CC14-treated rats. Methods Find Exp Clin Pharmacol. 23(2): 79-84.
Kim H P, Kim S Y, Lee E J, Kim Y C, Kim Y C. 1997. Zeaxanthin dipalmitate from Lycium chinense has hepatoprotective activity. Res. Comm. Mol. Path. Pharm. 97: 301-314.
Leist M, Gantner F, Bohlinger I, Tiegs G, Germann P G, Wendel A. 1995. Tumor necrosis factor-induced hepatocyte apoptosis precedes liver failure in experimental murine shock models. Am. J. Pathol. 146: 1220-1234.
Ding, Y. J. Chem. Soc., Perkin Trans. 1, 2000, 1651-1655.
Bertram, J. S., Pung, A., Churley, M., Kappock, T. J. I., Wilkins, L. R., and Cooney, R. V. (1991) Diverse carotenoids protect against chemically induced neoplastic transformation. Carcinogenesis, 12, 671-678.
Pung, A., Rundhaug, J. E., Yoshizawa, C. N., and Bertram, J. S. (1988) b-Carotene and canthaxanthin inhibit chemically- and physically-induced neoplastic transformation in 10T1/2 cells. Carcinogenesis, 9, 1533-1539.
Reznikoff, C. A., Bertram, J. S., Brankow, D. W., and Heidelberger, C. (1973) Quantitative and Qualitative Studies of Chemical Transformation of Cloned C3H Mouse Embryo Cells Sensitive to Postconfluence Inhibition of Cell Division. Cancer Research, 33, 2339-2349.
R. Merriman and J. S. Bertram. Reversible inhibition by retinoids of 3-methylcholanthrene-induced neoplastic transformation in C3H10T1/2 cells. Cancer Res. 39:1661-1666, 1979.
J. S. Bertram. Neoplastic Transformation in Cell Cultures: In Vitro/In-Vivo Correlations. IARC Sci. Pub. 67:77-91, 1985.
J. S. Bertram, M. Z. Hossain, A. Pung, and J. E. Rundhaug. Development of in vitro systems for chemoprevention research. Prev. Med. 18:562-575, 1989.
W. Aoi, Y. Naito, K. Sakuma, M. Kuchide, H. Tokuda, T. Maoka, S. Toyokuni, S. Oka, M. Yasuhura, and T. Toshikawa. Astaxanthin limits exercise-induced skeletal and cardiac muscle damage in mice. Antioxidants & Redox Signaling 5(l):139-144, 2003.
K. Ohgami, K. Shiratori, S. Kotake, T. Nishida, N. Mizuki, K. Yazawa, and S. Ohno. Effects of astaxanthin on lipopolysaccharide-induced inflammation in vitro and in vivo. Investigative Ophthalmology & Visual Science 44(6):2694-2701, 2003.
Barrett, T. D., Hennan, J. K., Marks, R. M., and Lucchesi, B. R. (2002). C-reactive protein associated increase in myocardial infarct size after ischemia/reperfusion. Journal of Pharmacology and Experimental Therapeutics 303(3): 1007-1013.
A. A. Spector, J A Gordon and S A Moore (1988) “Hydroxyeicosatetraenoic Acids (HETEs)”. Prog. Lipid res. vol 27, pp 271-323.
J M Drazen, E Isreal and P M O'Byrne (1999) “Treatment of Asthma with Drugs Modifying the Leukotriene Pathway”. New Engl. J. Med. vol. 340(3), pp 197-206.

1. A method of at least partially inhibiting the biological activity of 5-Lipoxygenase in a subject comprising administering to a subject an effective amount of a pharmaceutically acceptable formulation comprising a synthetic carotenoid analog or a carotenoid derivative;
wherein the carotenoid analog or derivative has the structure 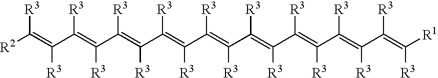
wherein each R³ is independently hydrogen or methyl;
wherein R¹ and R² are independently a cyclic ring comprising at least one substituent W or an acyclic group comprising at least one substituent W, wherein each cyclic ring is independently: 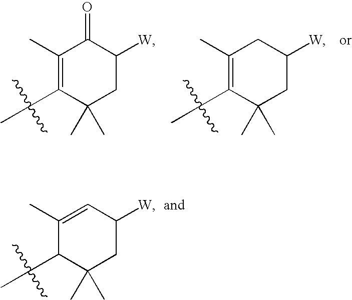
wherein the acyclic group has the structure 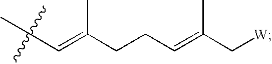
wherein W is 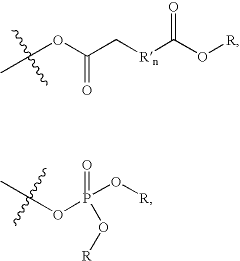
or a co-antioxidant; and
wherein each R is independently H, an alkyl group, an aryl group, a benzyl group, a Group IA metal, or a co-antioxidant, wherein R′ is CH₂, and wherein n ranges from 1 to 9.

2. The method of claim 1, wherein the carotenoid analog or derivative has the structure

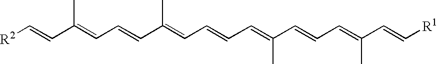

3. The method of claim 1, wherein the need for at least partially reduced biological activity of 5-Lipoxygenase is associated with treatment of the subject for prostate cancer.

4. The method of claim 1, wherein the need for at least partially reduced biological activity of 5-Lipoxygenase is associated with treatment of the subject for inflammation.

5. The method of claim 1, wherein the need for at least partially reduced biological activity of 5-Lipoxygenase is associated with treatment of the subject for asthma.

6. The method of claim 1, wherein the formulation is administered prior to the onset of an inflammatory response.

7. The method of claim 1, wherein when W or R is a co-antioxidant, the co-antioxidant is Vitamin C or a Vitamin C analog or a Vitamin C derivative.

8. The method of claim 0, wherein when W or R is a co-antioxidant, the co-antioxidant is Vitamin E, Vitamin E analogs, or Vitamin E derivatives.

9. The method of claim 0, wherein when W or R is a co-antioxidant, the co-antioxidant is a flavonoid, a flavonoid analog, or a flavonoid derivative.

10-11. (canceled)

12. The method of claim 1, wherein the aqueous dispersibility of the carotenoid analog or derivative is greater than about 5 mg/ml.

13-18. (canceled)

19. The method of claim 1, wherein the dosage of the carotenoid analog or derivative that is administered to the subject is in the range of about 10 mg/kg body weight to about 1000 mg/kg body weight.

20. The method of claim 1, wherein the formulation is adapted to be administered orally.

21. The method of claim 1, wherein the formulation is adapted to be administered parenterally.

22. (canceled)

23. The method of claim 1, wherein the formulation is administered to the subject parenterally, wherein the formulation comprises a dosage of the carotenoid analog or derivative in the range of about 0.25 mg to about 1.0 g per day.

24. (canceled)

25. (canceled)

26. The method of claim 1, wherein the formulation is adapted to be administered as an aqueous dispersion.

27. The method of claim 1, wherein the formulation is adapted to be administered intravenously.

28-31. (canceled)

32. The method of claim 1, wherein the formulation is adapted to be administered as an aerosol.

33. The method of claim 1, wherein the carotenoid analog or derivative is adapted to be administered in the form of an emulsion.

34. The method of claim 33, wherein the emulsion comprises water, oil and lecithin.

35. The method of claim 1, wherein the formulation comprises at least two different carotenoid analogs or derivatives.

36. The method of claim 1, wherein at least one substituent is 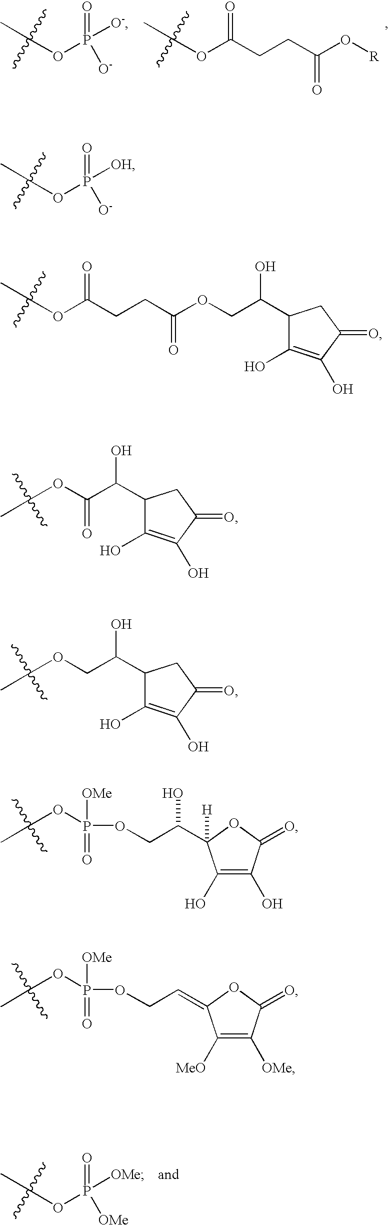
wherein each R is independently H, alkyl, aryl, benzyl, Group IA metal, or co-antioxidant.

37. (canceled)

38. (canceled)

39. The method of claim 1, wherein the carotenoid analog or derivative has the structure 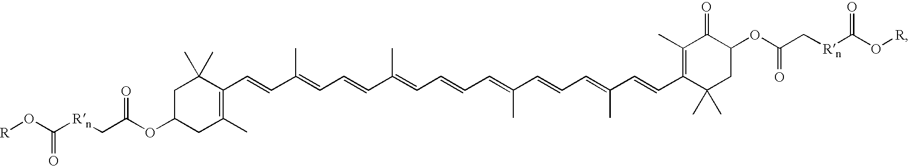
wherein R′ is CH₂, and wherein each R is independently H, alkyl, aryl, benzyl, Group IA metal, or co-antioxidant.

40. The method of claim 1, wherein the carotenoid analog or derivative has the structure 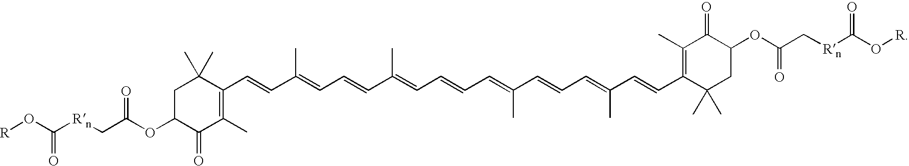
wherein R′ is CH₂, and wherein each R is independently H, alkyl, aryl, benzyl, Group IA metal, or co-antioxidant.

41. The method of claim 1, wherein the carotenoid analog or derivative has the structure

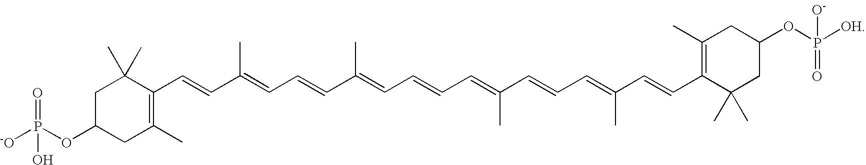

42. The method of claim 1, wherein the carotenoid analog or derivative has the structure

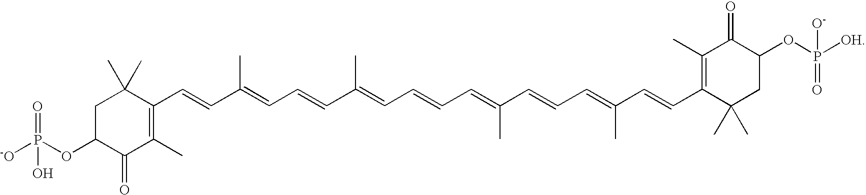

43. The method of claim 1, wherein the carotenoid analog or derivative has the structure

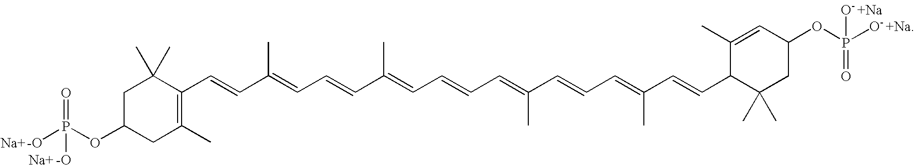

44. The method of claim 1, wherein the carotenoid analog or derivative has the structure

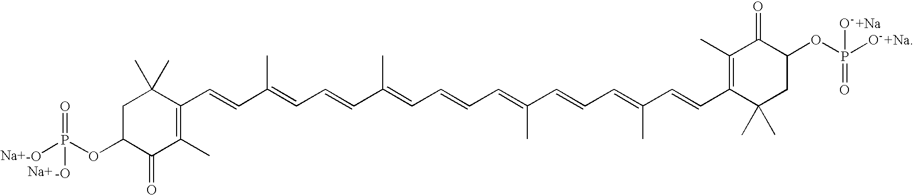

45. The method of claim 1, wherein the carotenoid analog or derivative has the structure

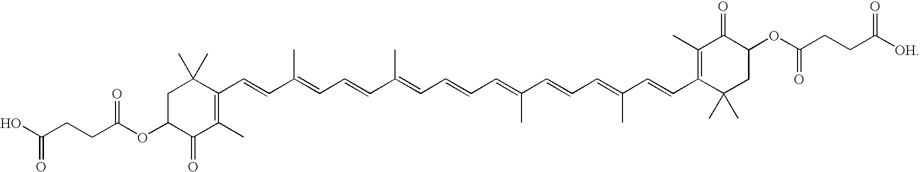

46. The method of claim 1, wherein the carotenoid analog or derivative has the structure

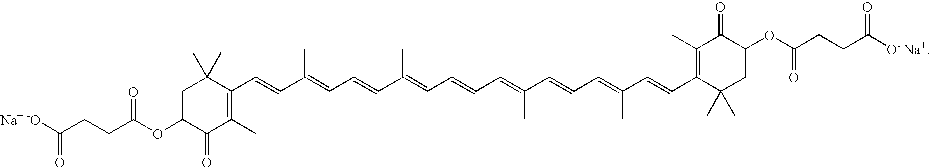

47. The method of claim 1, wherein the carotenoid analog or derivative has the structure

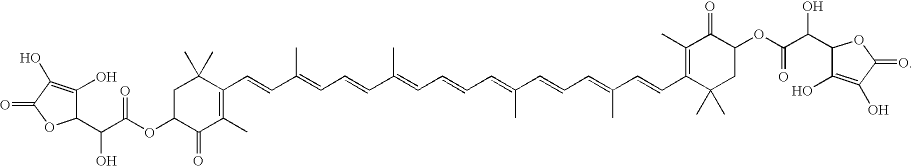

48. The method of claim 1, wherein the carotenoid analog or derivative has the structure

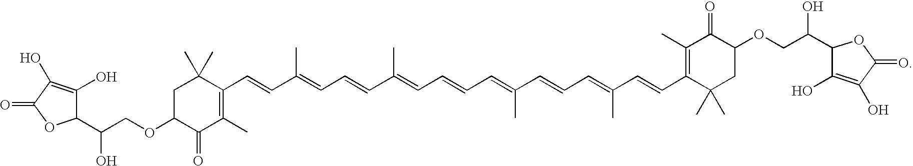

49. The method of claim 1, wherein the carotenoid analog or derivative has the structure

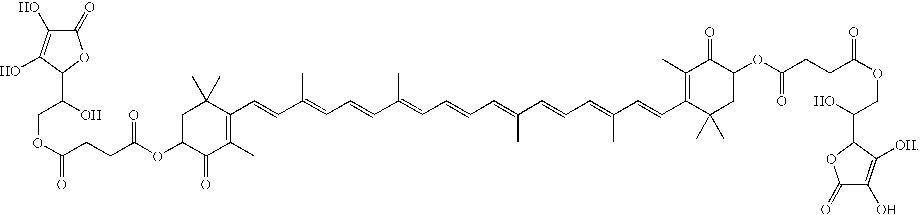

50. (canceled)

51. The method of claim 1, wherein the carotenoid analog or derivative has the structure

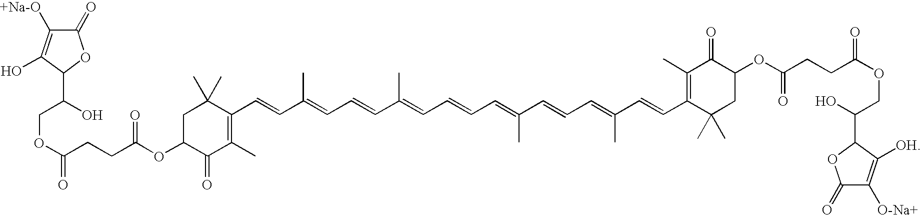

52. The method of claim 1, wherein the carotenoid analog or derivative has the structure

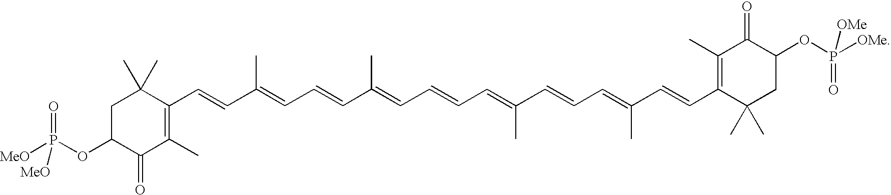

53. (canceled)

54. The method of claim 1, wherein the carotenoid analog or derivative has the structure

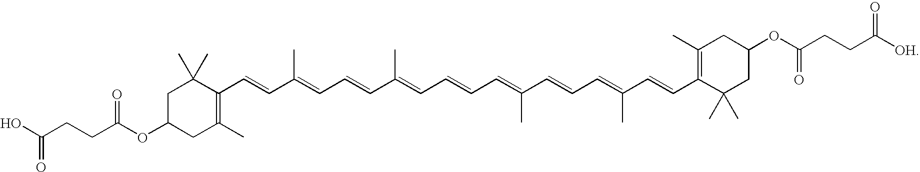

55. The method of claim 1, wherein the carotenoid analog or derivative has the structure

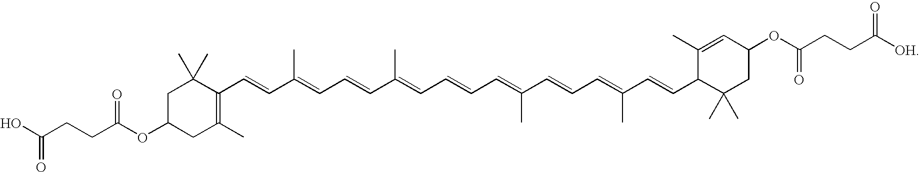

56. (canceled)

57. (canceled)

58. The method of claim 1, wherein the carotenoid analog or derivative has the structure 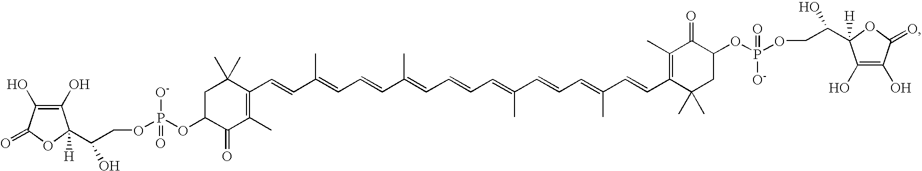
wherein the carotenoid analog or derivative further comprises one or more counterions.

59. The method of claim 1, wherein the carotenoid analog or derivative has the structure 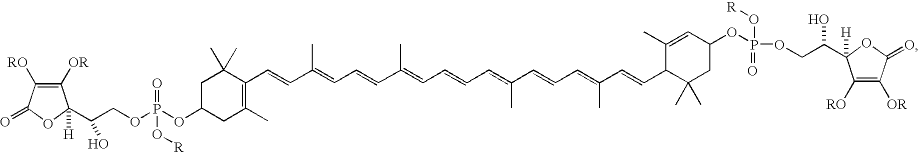
wherein each R is independently H, alkyl, aryl, benzyl, or Group IA metal.

60. The method of claim 1, wherein the carotenoid analog or derivative has the structure 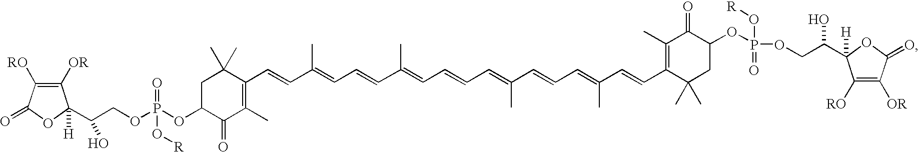
wherein each R is independently H, alkyl, aryl, benzyl, or Group IA metal.

61. The method of claim 1, wherein the carotenoid analog or derivative has the structure 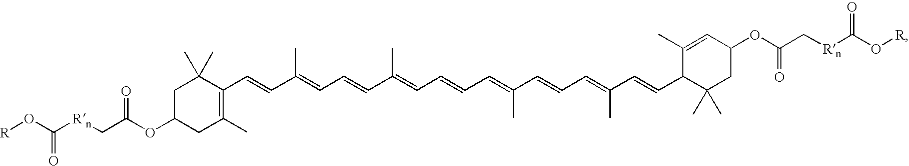
wherein each R is independently H, alkyl, aryl, benzyl, Group IA metal, or co-antioxidant.

62. The method of claim 1, wherein at least one W or at least one R from at least a portion of the carotenoid analog or derivative administered to the cells is cleaved during use, and wherein the cleavage product of the carotenoid analog or derivative is biologically active.

63. (canceled)

64. A method of at least partially inhibiting the activity of 5-Lipoxygenase comprising contacting 5-Lipoxygenase with a synthetic carotenoid analog or a carotenoid derivative;
wherein the carotenoid analog or derivative has the structure 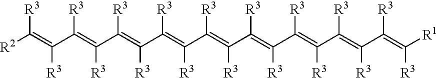
wherein each R³ is independently hydrogen or methyl;
wherein R¹ and R² are independently a cyclic ring comprising at least one substituent W or an acyclic group comprising at least one substituent W, wherein each cyclic ring is independently: 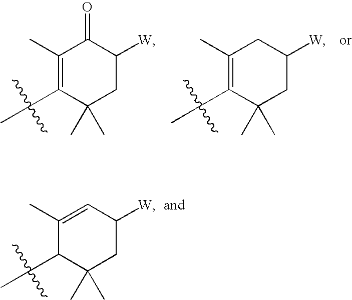
wherein the acyclic group has the structure 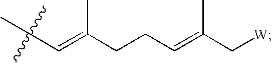
wherein W is 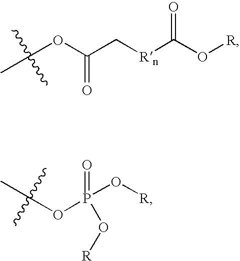
or a co-antioxidant; and
wherein each R is independently H, an alkyl group, an aryl group, a benzyl group, a Group IA metal, or a co-antioxidant, wherein R′ is CH₂, and wherein n ranges from 1 to 9.

65. The method of claim 64, wherein the carotenoid analog or derivative has the structure

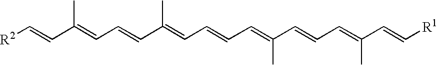

66. The method of claim 64, wherein the molar ratio of synthetic carotenoid analog or carotenoid derivative that is contacted with 5-Lipoxygenase about 1.0.

67. The method of claim 64, wherein the molar ratio of synthetic carotenoid analog or carotenoid derivative that is contacted with 5-Lipoxygenase is greater than about 1.0.

68. (canceled)

69. The method of claim 64, wherein the molar ratio of synthetic carotenoid analog or carotenoid derivative that is contacted with 5-Lipoxygenase is less than about 2.5.

70. A method of at least partially inhibiting the biological activity of a COX enzyme in a subject comprising administering to a subject an effective amount of a pharmaceutically acceptable formulation comprising a synthetic carotenoid analog or a carotenoid derivative;
wherein the carotenoid analog or derivative has the structure 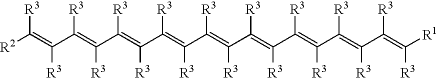
wherein each R³ is independently hydrogen or methyl;
wherein R¹ and R² are independently a cyclic ring comprising at least one substituent W or an acyclic group comprising at least one substituent W, wherein each cyclic ring is independently: 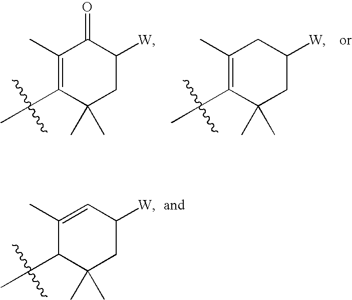
wherein the acyclic group has the structure 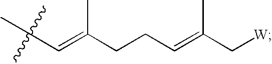
wherein W is 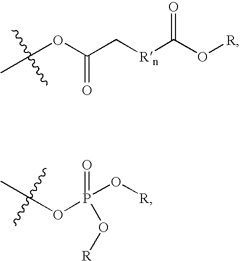
or a co-antioxidant; and
wherein each R is independently H, an alkyl group, an aryl group, a benzyl group, a Group IA metal, or a co-antioxidant, wherein R′ is CH₂, and wherein n ranges from 1 to 9.

71. The method of claim 70, wherein the carotenoid analog or derivative has the structure

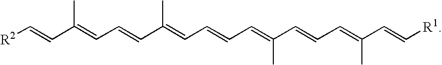

72. The method of claim 70, wherein the COX enzyme is COX-1.

73. The method of claim 70, wherein the COX enzyme is COX-2.

74. The method of claim 70, wherein the COX enzyme is a combination of COX-1 and COX-2.

75. The method of claim 70, wherein the inhibition of the COX enzyme is associated with the biological availability of 11-HETE.

76. The method of claim 70, wherein the inhibition of the COX enzyme is associated with the biological availability of PGF2α.

77-85. (canceled)
